SEQUENCE LISTING (6 sequences)

SEQ ID NO: 1 - DNA, 22 bp, Artificial Sequence
  Nucleic Acid
  Mus musculus
ctgcaggttctccacacctatg

SEQ ID NO: 2 - DNA, 21 bp, Artificial Sequence
  Nucleic Acid
  Mus musculus
gaatttgccgtgagtggagtc

SEQ ID NO: 3 - DNA, 25 bp, Artificial Sequence
  Nucleic Acid
  Mus musculus
gacaggatgcagaaggagattactg

SEQ ID NO: 4 - DNA, 21 bp, Artificial Sequence
  Nucleic Acid
  Mus musculus
acatctgctggaaggtggaca

SEQ ID NO: 5 - DNA, 22 bp, Artificial Sequence
  Nucleic Acid
  Helicobacter pylori
aggttaagaggatgcgtcagtc

SEQ ID NO: 6 - DNA, 23 bp, Artificial Sequence
  Nucleic Acid
  Helicobacter pylori
cgcatgatattcccattagcagt

(19) United States
(12) Patent Application Publication
(10) Pub. No.: US 2023/0133037 A1
(43) Pub. Date: May 4, 2023

(54) EXTENDED RELEASE GASTRORETENTIVE FORMULATION AGAINST HELICOBACTER PYLORI

(71) Applicant: SOLSTAR PHARMA, Laval (CA)

(72) Inventors: Tien Canh LE, Montreal (CA); Dionissios BALTZIS, Laval (CA); Max ARELLA, Laval (CA)

(21) Appl. No.: 17/561,782

(22) PCT Filed: Jun. 26, 2020
(86) PCT No.: PCT/CA2020/050885

§ 371 Date: Dec. 24, 2021

Related U.S. Application Data
(60) Provisional application No. 62/867,951, filed on Jun. 28, 2019.

(51) Int. Cl. A61K 31/357; A61K 45/06; A61K 31/4439; A61K 9/16

(52) U.S. Cl. CPC A61K 31/357; A61K 45/06; A61K 31/4439; A61K 9/1652; A61K 9/1611; A61K 9/1617

(57) ABSTRACT

The present document describes extended release gastro retentive dosage form comprising a carboxylated polysaccharide and cinnamaldehyde conjugate formed via an acetal, hemiacetal or cyclic hemiacetal formed between an aldehyde group of the cinnamaldehyde and a hydroxyl group of the carboxylated polysaccharide. The document also describes extended release gastro retentive dosage form comprising an artesunate emulsion having a pH value of from about 7.5 to 7.9 and comprising an artesunate or pharmaceutically acceptable salts thereof, and stereoisomers thereof stabilized with an emulsifying agent. The document also describes the use of the dosage forms for treatment of H. Pylori infection.

### CROSS-REFERENCE TO RELATED APPLICATIONS

This application claims priority of U.S. Provisional Patent Application No. 62/867,951 filed on Jun. 28, 2019, the specification of which is hereby incorporated by reference in its entirety.

### BACKGROUND

### (a) Field

The subject matter disclosed generally relates to extended release gastro retentive dosage forms. More specifically, the subject matter disclosed relates to extended release gastro retentive dosage forms comprising cinnamaldehyde and/or artesunate for the treatment of H. Pylori and/or cancer.

### (b) Related Prior Art

A wide range of medicinal and aromatic plants have been explored for their essential oils. These oils have great potential in the field of biomedicine as they effectively destroy a wide range of several bacterial, fungal, and viral pathogens. Essential oils are also employed in aromatherapy and for the treatment of several diseases including cardiovascular disease, diabetes, Alzheimer's and cancer. The presence of different types of aldehydes, phenolics, terpenes, and other antimicrobial compounds means that the essential oils are effective against a diverse range of pathogens.

Essentials oils (EO) are plant extracts that are hydrophobic and mainly composed of phenolic compounds which act on the bacterial membrane by disruption it. Many studies demonstrated that EO such as oregano, cinnamon, thyme oils have an important antibacterial activity against E. coli, L. monocytogenes, Salmonella Typhimurium, Staphylococcus aureus, Clostridium perfringens and C. botulinum. They also demonstrated that these EO have a strong bactericidal activity against Campylobacter jejuni, E. coli 0157:H7, L. monocytogenes and S. enterica.

Recently, the prevalence of antimicrobial drug resistance has prompted researchers to discover novel antimicrobial lead molecules to treat various human pathogens. Some of the presently available synthetic drugs fail to inhibit many pathogens. In addition, the use of synthetic chemicals for the control of pathogenic microorganisms is limited because of their carcinogenic effects, acute toxicity, and environmental hazard potential. In this regard, the exploitation of essential oils to control epidemic multidrug-resistant of pathogenic microorganisms can be useful to combat various infectious diseases.

EO are hydrophobic and highly volatile components derived from a great range of different chemical classes, EO are known to be susceptible to conversion and degradation reactions by several parameters, particularly temperature, light, and oxygen, etc. are recognized to have a crucial impact on EO integrity. Auto-oxidation and polymerization processes may result in a loss of quality and pharmacological properties. Furthermore, their oil liquid form limit its application in pharmaceutic. For this reason, there are needs for solutions to protect EO and preserve their stability, especially during storage conditions.

The present invention proposes to present a solution for the preservation and stabilization of essential oil compounds, particularly of cinnamaldehyde, with microencapsulated systems. The present invention hopes to mitigate the shortcomings of other microencapsulation systems.

The present invention proposes a conjugation on cinnamaldehyde with a carboxylated polysaccharide to stabilize cinnamaldehyde, thus preserving the chemical properties and preventing or reducing evaporation and degradation.

SUMMARY

According to an embodiment, there is provided an extended release gastro retentive dosage form comprising:

a carboxylated polysaccharide and cinnamaldehyde conjugate, the conjugate formed via an acetal, hemiacetal or cyclic hemiacetal formed between an aldehyde group of the cinnamaldehyde and a hydroxyl group of the carboxylated polysaccharide.

The carboxylated polysaccharide may be a carboxymethyl cellulose, a carboxymethyl starch, a carboxymethyl high amylose starch, a carboxyethyl starch, a carboxyethyl high amylose starch, a succinyl-starch, a succinyl high amylose starch, a carboxymethyl guar gum, a carboxymethyl hydroxypropyl guar gum, a gellan gum, a xanthan gum, an alginate, a pectate, a hyaluronate, or combinations thereof.

The carboxylated polysaccharide may be carboxymethyl cellulose, carboxymethyl starch, or a combination thereof.

The carboxylated polysaccharide may be of general formula (I), or pharmaceutically acceptable salts thereof, and stereoisomers thereof:

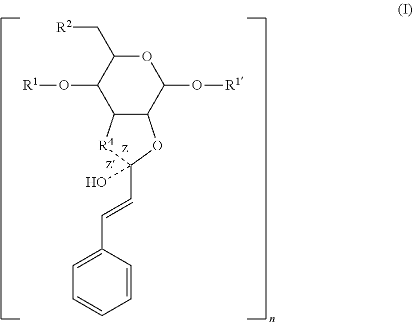

wherein
n=15−1.67×10⁵;
R¹ and R^{1′} are each independently

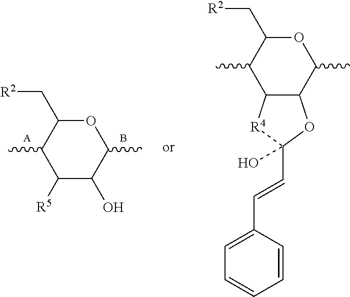

R² is OH, R¹, O—(CH₂)ₘCO₂R³, O—(CH₂)ₘ(CO₂R³)(CH₂)ₚCH₃, O—CO(CH₂)ₘ(CO₂R³)(CH₂)ₚCH₃, O—CO(CH₂)ₘCO₂R³, O—CO—CH₂—CH—COR³, or O—CO—CH₂—CH(CH₂)ₚ—COR³;
R³ is absent or H;
R⁴ is O when bond Z is present and R⁵ when bond Z is absent;
bond Z′ is present when bond Z is absent;
R⁵ is OH or R¹;
m=0-4;
p=0-8; and
[symbol] indicates a covalent bond connecting to said R^{1′}, and [symbol] indicates a covalent bond connecting to the R¹.

The carboxylated polysaccharide of general formula (I), may be of general formula (Ia):

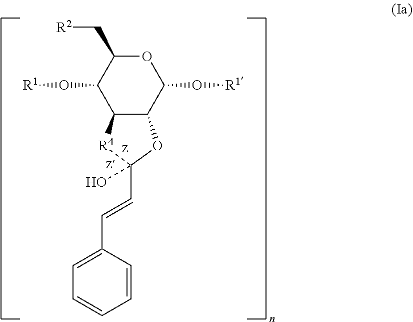

wherein
n=15−1.67×10⁵;
R¹ and R^{1′} are each independently

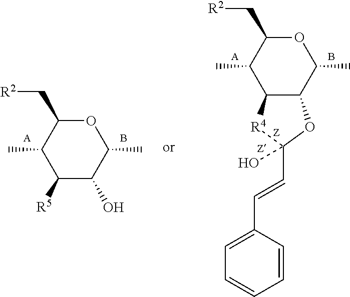

R² is OH, R¹, O—(CH₂)ₘCO₂R⁴, O—(CH₂)ₘ(CO₂R³)(CH₂)ₚCH₃, O—CO(CH₂)ₘ (CO₂R³)(CH₂)ₚCH₃, O—CO(CH₂)ₘ CO₂R³, O—CO—CH—COR³, or O—CO—CH₂—CH(CH₂)ₚ—COR³;
R³ is absent or H;
m=0-4;
p=0-8;
R⁴ is O when bond Z is present and R⁵ when bond Z is absent;
bond Z′ is present when bond Z is absent;
R⁵ is OH or R¹; and
[symbol] indicates a covalent bond connecting to the R^{1′}, and [symbol] indicates a covalent bond connecting to the R¹.

The R² may be O—CH₂COO⁻.

The degree of substitution of the carboxyl containing group may be of from about 0.01 to about 1.0.

The degree of substitution may be from about 0.2 to about 0.4.

The degree of substitution may be 0.4.

The cinnamaldehyde may have a degree of substitution on the carboxylated polymer of from about 0.01 to about 0.20.

According to another embodiment, there is provided an extended release gastro retentive dosage form comprising an artesunate emulsion having a pH value of from about 7.5 to 7.9 and comprising an artesunate or pharmaceutically acceptable salts thereof, and stereoisomers thereof stabilized with an emulsifying agent.

The weight ratio of the artesunate pharmaceutically acceptable salt and the emulsifying agent in the artesunate emulsion may be from about 9:1 to about 1:1.

The weight ratio may be 3:2.

The emulsifying agent may be a surfactant.

The surfactant may be a nonionic, an anionic, a cationic, an amphoteric surfactant, or a combination thereof.

The surfactant may be selected from the group consisting of sodium lauryl sulfate, sorbitan stearate, sorbitan esters, sodium laureth sulfate, sarkosyl, cocamidopropyl betaine (CAPB), sodium lauryl ether sulfonate, alkyl benzene sulfonates, nonylphenol ethoxylate, hexadecylbetaine, lauryl betaine, and ether ethoxylate, Sodium Myristyl sulfate, polysorbate 20, polysorbate 80, lecithin, Octyl phenol ethoxylate (Triton X-100), glyceryl monostearate, 3-[(3-Cholamidopropyl)dimethylammonio]-1-propanesulfonate (CHAPS). The surfactant may be preferably sodium lauryl sulfate.

The emulsifying agent may be a cholate. The cholate may be selected from the group consisting of cholic acid, glycocholic acid, taurocholic acid, deoxycholic acid, chenodeoxycholic acid, glycochenodeoxycholic acid, taurochenodeoxycholic acid and lithocholic acid.

The pH value may be obtained with a weak base. The weak base may be a carbonate salt. The carbonate salt may be selected from the group consisting of sodium carbonate (Na₂CO₃), potassium carbonate (K₂CO₃), sodium bicarbonate (NaHCO₃), and potassium and bicarbonate (KHCO₃).

The extended release gastro retentive dosage form may further comprise a proton pump inhibitor. The proton pump inhibitor may be omeprazole, lansoprazole, dexlansoprazole, rabeprazole, pantoprazole, esomeprazole, omeprazole, or combinations thereof.

The extended release gastro retentive dosage may further comprise an antibiotic. The antibiotic may be amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

The extended release gastro retentive dosage form of the present invention may comprise the carboxylated polysaccharide and cinnamaldehyde conjugate, and further comprising the extended release gastro retentive dosage form comprising the artesunate emulsion according to the present invention.

The extended release gastro retentive dosage form may be for use for the prevention or treatment of Helicobacter pylori infection, gastric ulcers, or a combination thereof.

The extended release gastro retentive dosage form for use may further comprise the use of a proton pump inhibitor. The proton pump inhibitor may be omeprazole, lansoprazole, dexlansoprazole, rabeprazole, pantoprazole, esomeprazole, omeprazole, or combinations thereof.

The extended release gastro retentive dosage form for use may further comprise an antibiotic. The antibiotic may be amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

In the extended release gastro retentive dosage form of the present invention, the salts thereof may be pharmaceutically acceptable salts thereof.

According to another embodiment, there is provided a method for prevention or treatment of Helicobacter pylori infection, gastric ulcers, or a combination thereof comprising administering to a subject in need thereof a therapeutically effective amount of the extended release gastro retentive dosage form of the present invention.

According to another embodiment, there is provided a method for the prevention or treatment of Helicobacter pylori infection, gastric ulcers, gastric cancer or a combination thereof comprising administering to a subject in need thereof a therapeutically effective amount of the extended release gastro retentive dosage form of the present invention.

According to another embodiment, there is provided a method for the prevention or treatment of Helicobacter pylori infection, gastric ulcers, gastric cancer or a combination thereof comprising administering to a subject in need thereof a therapeutically effective amount of the extended release gastro retentive dosage form of the present invetion.

The method may further comprise administering to the subject in need thereof a therapeutically effective amount of a proton pump inhibitor. The proton pump inhibitor may be omeprazole, lansoprazole, dexlansoprazole, rabeprazole, pantoprazole, esomeprazole, omeprazole, or combinations thereof.

The method may further comprise administering to the subject in need thereof a therapeutically effective amount of an antibiotic. The antibiotic may be amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

The proton pump inhibitor may be administered before, or at the same time as the extended release gastro retentive dosage form.

The proton pump inhibitor may be administered before the extended release gastro retentive dosage form.

The proton pump inhibitor may be administered about 10 mins to about 60 minutes before the extended release gastro retentive dosage form.

The proton pump inhibitor may be administered about 20 mins to about 30 minutes before the extended release gastro retentive dosage form.

The proton pump inhibitor may be administered about 30 minutes before the extended release gastro retentive dosage form.

The antibiotic may be administered before, after, or at the same time as the extended release gastro retentive dosage form.

The antibiotic may be administered at the same time as the extended release gastro retentive dosage form.

The proton pump inhibitor may be administered before the extended release gastro retentive dosage form, and the antibiotic may be administered at the same time or after the extended release gastro retentive dosage form.

According to another embodiment, there is provided a use of an extended release gastro retentive dosage form of the present invention for the prevention or treatment of Helicobacter pylori infection, gastric ulcers, or a combination thereof in a subject in need thereof.

According to another embodiment, there is provided a use of an extended release gastro retentive dosage form of the present invention for the prevention or treatment of Helicobacter pylori infection, gastric ulcers, gastric cancer or a combination thereof in a subject in need thereof.

According to another embodiment, there is provided a use of an extended release gastro retentive dosage form of the present invention for the preparation of a medicament for the prevention or treatment of Helicobacter pylori infection, gastric ulcers, or a combination thereof in a subject in need thereof.

According to another embodiment, there is provided a use of an extended release gastro retentive dosage form of the present invention for the preparation of a medicament for the prevention or treatment of Helicobacter pylori infection, gastric ulcers, gastric cancer or a combination thereof in a subject in need thereof.

The use may further comprising the use of a proton pump inhibitor. The proton pump inhibitor may be omeprazole, lansoprazole, dexlansoprazole, rabeprazole, pantoprazole, esomeprazole, omeprazole, or combinations thereof.

The proton pump inhibitor may be for use before, or at the same time as the extended release gastro retentive dosage form.

The proton pump inhibitor may be for use before the extended release gastro retentive dosage form.

The proton pump inhibitor may be for use about 10 mins to about 60 minutes before the extended release gastro retentive dosage form.

The proton pump inhibitor may be for use about 20 mins to about 30 minutes before the extended release gastro retentive dosage form.

The proton pump inhibitor may be for use about 30 minutes before the extended release gastro retentive dosage form.

The use may further comprise an antibiotic.

The antibiotic may be amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

The antibiotic may be for use before, after, or at the same time as the extended release gastro retentive dosage form.

The antibiotic may be for use at the same time as the extended release gastro retentive dosage form.

The proton pump inhibitor may be for use before the extended release gastro retentive dosage form, and the antibiotic may be administered at the same time or after the extended release gastro retentive dosage form.

According to another embodiment, there is provided a process for the preparation of an extended release gastro retentive formulation comprising an artesunate emulsion comprising the steps of
a) introducing an artesunate or pharmaceutically acceptable salts thereof, or stereoisomers thereof in a buffered solution comprising an emulsifying agent, to obtain an emulsified artesunate solution;
b) adjusting pH value of the emulsified artesunate solution to a pH value of about 7.5 to about 7.9, to obtain an adjusted emulsified artesunate solution; and
c) drying the adjusted emulsified artesunate solution, to obtain a dry powder of emulsified artesunate, having a pH value of from about 7.5 to 7.9.

The pH value of the dry powder of emulsified artesunate may be about 7.75.

The weight ratio of the artesunate pharmaceutically acceptable salt and the emulsifying agent may be from about 9:1 to about 1:1.

The weight ratio may be 3:2.

The emulsifying agent may be a surfactant.

The surfactant may be a nonionic, an anionic, a cationic, an amphoteric surfactant, or a combination thereof.

The surfactant may be selected from the group consisting of sodium lauryl sulfate, sorbitan stearate, sorbitan esters, sodium laureth sulfate, sarkosyl, cocamidopropyl betaine (CAPB), sodium lauryl ether sulfonate, alkyl benzene sulfonates, nonylphenol ethoxylate, hexadecylbetaine, lauryl betaine, and ether ethoxylate, Sodium Myristyl sulfate, polysorbate 20, polysorbate 80, lecithin, Octyl phenol ethoxylate (Triton X-100), glyceryl monostearate, 3-[(3-Cholamidopropyl)dimethylammonio]-1-propanesulfonate (CHAPS). The surfactant may be preferably sodium lauryl sulfate.

The emulsifying agent may be a cholate. The cholate may be selected from the group consisting of cholic acid, glycocholic acid, taurocholic acid, deoxycholic acid, chenodeoxycholic acid, glycochenodeoxycholic acid, taurochenodeoxycholic acid and lithocholic acid.

The pH value may be obtained with a weak base. The weak base may be a carbonate salt. The carbonate salt may be selected from the group consisting of sodium carbonate (Na₂CO₃), potassium carbonate (K₂CO₃), sodium bicarbonate (NaHCO₃), potassium and bicarbonate (KHCO₃), and combinations thereof.

The drying may be by spray drying

The following terms are defined below.

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “subject” is intended to mean humans and non-human mammals such as primates, cats, dogs, swine, cattle, sheep, goats, horses, rabbits, rats, mice and the like.

As used herein, the term “compound” or “compound of the present invention” is intended to mean the conjugation complex and/or the complex described herein.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “therapeutically effective amount” is intended to mean an amount of a compound of Formula I which, when administered to a subject is sufficient to effect treatment for a disease-state associated with insufficient apoptosis. The amount of the compound of Formula I will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression of the condition.

As used herein, the term “treating cancer” is intended to mean the administration of a pharmaceutical composition of the present invention to a subject, preferably a human, which is afflicted with cancer to cause an alleviation of the cancer by killing, inhibiting the growth, or inhibiting the metastasis of the cancer cells.

As used herein, the term “preventing disease” is intended to mean, in the case of cancer, the post-surgical, post-chemotherapy or post-radiotherapy administration of a pharmaceutical composition of the present invention to a subject, preferably a human, which was afflicted with cancer to prevent the regrowth of the cancer by killing, inhibiting the growth, or inhibiting the metastasis of any remaining cancer cells. Also included in this definition is the prevention of prosurvival conditions that lead to diseases such as asthma, MS and the like.

As used herein, the term “synergistic effect” is intended to mean that the effect achieved with the combination of the compounds of the present invention and either the chemotherapeutic agents or death receptor agonists of the invention is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “IC₅₀” is intended to mean an amount, concentration or dosage of a particular compound of the present invention that achieves a 50% inhibition of a maximal response, such as displacement of maximal fluorescent probe binding in an assay that measures such response.

As used herein, the term “EC₅₀” is intended to mean an amount, concentration or dosage of a particular compound of the present invention that achieves a 50% inhibition of cell survival.

The terms “degree of substitution” or “degree of coordination” is intended to mean the average number of substituents per sugar unit (SU), the monomer unit of the polysaccharides used in the invention. Since each SU contains three hydroxyl groups, the DS can vary between 0-3.

As used herein the term “carboxylated polysaccharide” is intended to mean a carboxymethyl cellulose, a carboxymethyl starch (starch glycolate), a carboxymethyl high amylose starch, a carboxyethyl starch, a carboxyethyl high amylose starch, a succinyl-starch, a succinyl high amylose starch carboxymethyl guar gum, a carboxymethyl hydroxypropyl guar gum, a gellan gum, a xanthan gum, an alginate, a pectate, a hyaluronate, or combinations thereof. Preferably the carboxylated polysaccharide that may be used in the present invention are carboxymethyl cellulose and/or carboxymethyl starch. Most preferably, the carboxylated polysaccharide is carboxymethyl starch. Also encompassed are combinations of hexose monosaccharides (allose, altrose, glucose, mannose, gulose, idose, galactose, and talose) in any suitable combinations, comprising an appropriate carboxylation modification.

The compounds of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)—or (S)—or, as (D)- or (L)- for amino acids. The present invention is intended to include all such possible isomers, as well as, their racemic and optically pure forms. Optically active (+) and (−), (R)—and (S)—, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

The compounds (in the form of the conjugation complex or the complex) of the present invention, or their salts, pharmaceutically acceptable salts or their prodrugs, may be administered in pure form or in an appropriate pharmaceutical composition, and can be carried out via any of the suitable accepted modes of Galenic pharmaceutical practice.

The pharmaceutical compositions of the present invention can be prepared by admixing a compound of the present invention with an appropriate pharmaceutically acceptable carrier, diluent or excipient, and may be formulated into preparations in solid, semi-solid, liquid form, such as tablets, capsules, powders, granules, solutions, suppositories, injections, gels, and microspheres. Typical routes of administering such pharmaceutical compositions of the present invention include oral. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of a disease-state as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an inhalable atomization or nebulization.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains between 0.01 to 1% by weight of the compound of the present invention.

The pharmaceutical composition of the present invention may include various materials, which convert the physical form of an oil liquid into solid powder form. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical compositions of the present invention may be prepared by methodology well known in the pharmaceutical art. For example, a pharmaceutical composition intended to be administered by injection can be prepared by admixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound of the present invention, or a pharmaceutically acceptable salt thereof.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates a schematic representation explaining the extended release of cinnamaldehyde from the gastro-retention tablet to the H. pylori infection site.

FIG. 2 illustrates the conjugation of cinnamaldehyde on starch glycolate leading to the formation of cyclic hemiacetal. The illustration only provides one of the several possible conjugation scenarios possible with starch glycolate, and is non-limiting.

FIG. 3 illustrates the FTIR analysis of a cinnamaldehyde conjugated with starch glycolate according to an embodiment of the present invention.

FIG. 4 illustrates scanning electron microscopy (SEM) with magnification 150× and 500× of the cinnamaldehyde conjugated with starch glvcolate.

FIG. 5 illustrates FTIR spectra of starch glycolate, untreated artesunate and artesunate/Starch Glycolate complex.

FIG. 6 illustrates SEM of granules of starch glycolate and artesunate/starch glycolate complex.

FIG. 7 illustrates acid-catalyzed hydrolysis of artesunate during gastric transit.

FIG. 8 illustrates artesunate (5%) with different treatments in simulated gastric fluid (SGF) after 2 and 12 h incubation at 36.5° C.

FIG. 9 illustrates FTIR spectra for untreated artesunate, artesunate/starch glycolate complex and water-soluble artesunate.

FIG. 10 illustrates SEM of granules of starch glycolate, artesunate/starch glycolate complex and water-soluble artesunate.

FIG. 11 illustrates agar disk-diffusion test for cinnamaldehyde/starch glycolate (CACINN) conjugate and artesunate/starch glycolate complex against different strains of Helicobacter Pylori. The strain shown is Helicobacter Pylori Ccug 17874.

FIG. 12 illustrates agar disk-diffusion test for cinnamaldehyde/starch glycolate conjugate and artesunate/starch glycolate complex against different strains of Helicobacter Pylon. The strain shown is Helicobacter Pylori 4038.

FIG. 13 illustrates agar disk-diffusion test for cinnamaldehyde/starch glycolate conjugate and artesunate/starch glycolate complex against different strains of Helicobacter Pylon. The strain shown is Helicobacter Pylori 3992.

FIG. 14 illustrates agar disk-diffusion test for cinnamaldehyde/starch glycolate conjugate and artesunate/starch glycolate complex against the strain Campylobacter jejuni CiP702.

FIG. 15 illustrates agar disk-diffusion test for cinnamaldehyde/starch glycolate conjugate and artesunate/starch glycolate complex against the strain Campylobacter coli.

FIG. 16 illustrates the Minimal Inhibition Concentration (MIC) determination of cinnamaldehyde/starch glycolate complex against helicobacter pylori.

FIG. 17 illustrates the Minimal Inhibition Concentration (MIC) determination of artesunate (top) and artesunate/starch glycolate conjugation complex (bottom) against Helicobacter pylori.

FIG. 18 illustrates antibacterial activity of artesunate with different treatments.

FIG. 19A illustrates dihydroartemisinin detected in the blood after intravenous administration 3.5 mg/kg in mice of artesunate untreated and that complexed with starch glycolate.

FIG. 19B illustrates dihydroartemisinin-glucuronide detected in the blood after intravenous administration 3.5 mg/kg in mice of artesunate untreated and that complexed with starch glycolate.

FIG. 20A illustrates dihydroartemisinin detected in the blood after oral administration 200 mg/kg in mice of artesunate untreated and that complexed with starch glycolate.

FIG. 20B illustrates dihydroartemisinin-glucuronide detected in the blood after oral administration 200 mg/kg in mice of artesunate untreated and that complexed with starch glycolate.

FIG. 21A illustrates dihydroartemisinin detected in the brain after intravenous administration 3.5 mg/kg in mice of artesunate untreated and that complexed with starch glycolate.

FIG. 21B illustrates dihydroartemisinin-glucuronide detected in the brain after intravenous administration 3.5 mg/kg in mice of artesunate untreated and that complexed with starch glycolate.

FIG. 22A illustrates dihydroartemisinin (detected in the brain after oral administration 200 mg/kg in mice of artesunate untreated and that complexed with starch glycolate.

FIG. 22B illustrates dihydroartemisinin-glucuronide detected in the brain after oral administration 200 mg/kg in mice of artesunate untreated and that complexed with starch glycolate.

FIG. 23A illustrates dihydroartemisinin detected in the liver after intravenous administration 3.5 mg/kg in mice of artesunate untreated and that complexed with starch glycolate.

FIG. 23B illustrates dihydroartemisinin-glucuronide detected in the liver after intravenous administration 3.5 mg/kg in mice of artesunate untreated and that complexed with starch glycolate.

FIG. 24A illustrates dihydroartemisinin detected in the liver after oral administration 200 mg/kg in mice of artesunate untreated and that complexed with starch glycolate.

FIG. 24B illustrates dihydroartemisinin-glucuronide detected in the liver after oral administration 200 mg/kg in mice of artesunate untreated and that complexed with starch glycolate.

FIG. 25A illustrates dihydroartemisinin detected in the prostate after intravenous administration 3.5 mg/kg in mice of artesunate untreated and that complexed with starch glycolate.

FIG. 25B illustrates dihydroartemisinin-glucuronide detected in the prostate after intravenous administration 3.5 mg/kg in mice of artesunate untreated and that complexed with starch glycolate.

FIG. 26A illustrates dihydroartemisinin detected in the prostate after oral administration 200 mg/kg in mice of artesunate untreated and that complexed with starch glycolate.

FIG. 26B illustrates dihydroartemisinin-glucuronide detected in the prostate after oral administration 200 mg/kg in mice of artesunate untreated and that complexed with starch glycolate.

FIG. 27 illustrates pharmacokinetic profile of water-soluble artesunate compared with commercial artesunate.

FIG. 28A illustrates antibacterial activity detected in mice by quantitative culture (from different components in the absence of Proton Pump Inhibitor (pantoprazole, 150 mg/kg).

FIG. 28B illustrates antibacterial activity detected in mice by quantitative PCR from different components in the absence of Proton Pump Inhibitor (pantoprazole, 150 mg/kg).

FIG. 29A illustrates antibacterial activity detected in mice by quantitative culture from different components in the presence of Proton Pump Inhibitor (pantoprazole, 150 mg/kg).

FIG. 29B illustrates antibacterial activity detected in mice by quantitative PCR from different components in the presence of Proton Pump Inhibitor (pantoprazole, 150 mg/kg).

FIG. 30A illustrates antibacterial activity detected in mice by quantitative culture (from different components in the presence of Proton Pump Inhibitor (pantoprazole, 150 mg/kg).

FIG. 30B illustrates antibacterial activity detected in mice by quantitative PCR from different components in the presence of Proton Pump Inhibitor (pantoprazole, 150 mg/kg).

FIG. 31 illustrates antibacterial activity of water-soluble artesunate detected in mice in the presence of Proton Pump Inhibitor (pantoprazole, 150 mg/kg).

FIG. 32 illustrates the effect of artesunate and an artesunate complex with starch glycolate according to the present invention, on the growth rate of 11 acute myeloid leukemia (AML) cell lines (N=3); Data was normalized to the control at day 3 for each cell line.

FIG. 33 illustrates the effect of artesunate and an artesunate complex with starch glycolate according to the present invention, on the viability of 11 acute myeloid leukemia (AML) cell lines (N=3); Data was normalized to the control at day 0 for each cell line.

FIG. 34 illustrates an artesunate/SG complex stability test. A fresh solution of ARTE/SG (prepared at day 0) was tested alongside three older solutions prepared 11, 15 or 18 days before the experiment. artesunate (ARTE) was used a positive control. Statistical analysis was performed between cell replicate.

FIG. 35 illustrates the methanol toxicity of select cell lines.

FIG. 36 illustrates the IC₅₀ of an artesunate/Starch Glycolate complex (ARTE.SG) and artesunate (ARTE) on 4 leukemic cell lines.

FIG. 37 illustrates the effect of a cinnamaldehyde/starch glycolate (CACINN) conjugation complex on the proliferation of AML cells. Data are reported as percentage of viability normalized to the control at day 3. Parametric one-way Anova was used for multiple comparisons followed by BH-adjusted t-test comparing NACINN conjugation complex -treated conditions to the control for each cell line.

FIG. 38 illustrates the in vitro dissolution profiles of Water-Soluble Artesunate in 1 L of simulated gastric fluid (pH 1.5) at 37° C. and 100 rpm, with a dissolution device Distek.

FIG. 39 illustrates the in vitro dissolution profiles of Cinnamaldehyde in 1 L of simulated gastric fluid (pH 1.5) at 37° C. and 100 rpm, with a dissolution device Distek.

It will be noted that throughout the appended drawings, like features are identified by like reference numerals.

### DETAILED DESCRIPTION

In embodiments there is disclosed an extended release gastro retentive dosage form comprising a carboxylated polysaccharide and cinnamaldehyde conjugate, the conjugate being formed via an acetal, hemiacetal, or cyclic hemiacetal formed between the aldehyde group of the cinnamaldehyde and a hydroxyl group of the carboxylated polysaccharide.

In another embodiment, there is disclosed an extended release gastro retentive dosage form comprising a carboxylated polysaccharide and artesunate complex, the complex being formed via coordination of a divalent metal cation between a carboxyl group of artesunate and a carboxylate group of the carboxylated polysaccharide.

The conjugation and/or stabilization by emulsion is to enhance the solubility of cinnamaldehyde/artesunate in aqueous medium, particularly in gastric fluid. It also improves the stability of cinnamaldehyde/artesunate, particularly in the case of the essential oil extracts that are highly volatile. The conjugation and/or stabilization by emulsion also converts an oil liquid into a solid powder form which is easy to formulate under different dosage forms (i.e. tablet; capsule; suppository; etc.), and finally it provides the mucoadhesive properties which are an asset to formulate the gastro-retention formulation. After ingestion and reaching the stomach, the tablet is hydrated the gastric acid medium which triggers the release of cinnamaldehyde/artesunate from protonation of the complexes.

### Cinnamaldehyde Conjugate

Cinnamaldehyde is an organic compound with the formula C₆H₅CH═CHCHO having the structure:

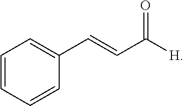

Occurring naturally as predominantly the trans (E) isomer, it gives cinnamon its flavor and odor. It is a phenylpropanoid that is naturally synthesized by the shikimate pathway. This pale yellow, viscous liquid occurs in the bark of cinnamon trees and other species of the genus Cinnamomum. The essential oil of cinnamon bark is about 55-76% cinnamaldehyde.

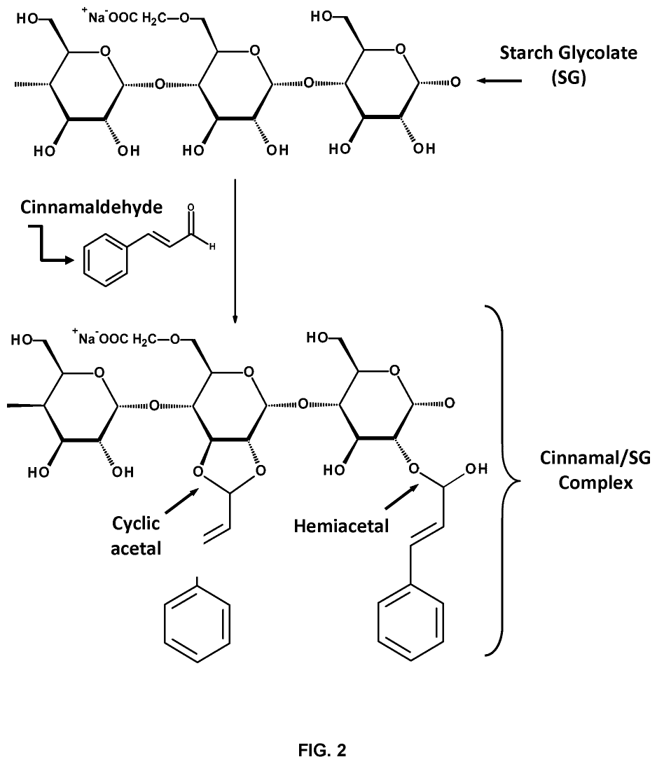

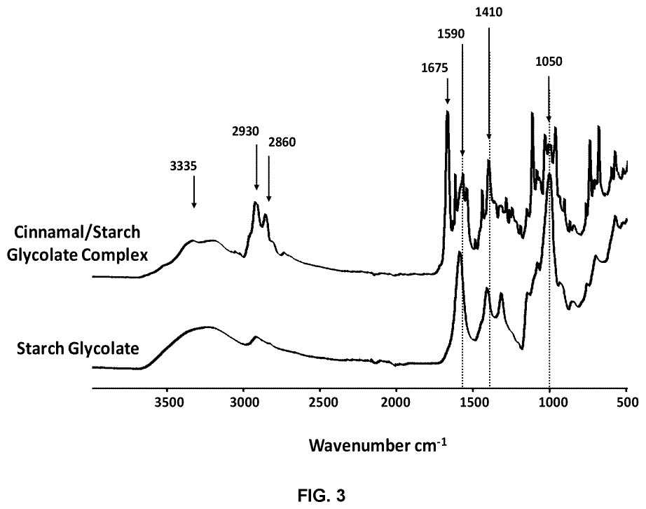

The conjugate of the present invention is prepared by reacting cinnamaldehyde with carboxylated polysaccharide to form an acetal, a hemiacetal or a cyclic hemiacetal. The reaction is carried out between the aldehyde group of cinnamaldehyde and hydroxyl groups of the carboxylated polysaccharide, as illustrated in FIG. 2, as well as herein below.

In embodiments, the carboxylated polysaccharide that may be used in the present invention include a carboxymethyl cellulose, a carboxymethyl starch (starch glycolate), a carboxymethyl high amylose starch, a carboxyethyl starch, a carboxyethyl high amylose starch, a succinyl-starch, a succinyl high amylose starch, starch sodium octenyl succinate, high amylose starch sodium octenyl succinate , a carboxymethyl guar gum, a carboxymethyl hydroxypropyl guar gum, a gellan gum, a xanthan gum, an alginate, a pectate, a hyaluronate, or combinations thereof. Preferably the carboxylated polysaccharide that may be used in the present invention are carboxymethyl cellulose and/or carboxymethyl starch. Most preferably, the carboxylated polysaccharide is carboxymethyl starch.

According to an embodiment, the degree of substitution of the carboxyl containing group of the carboxylated polysaccharide may be from about 0.4 to about 1.0, or from about 0.5 to about 1.0, or from about 0.6 to about 1.0, or from about 0.7 to about 1.0, or from about 0.8 to about 1.0, or from about 0.9 to about 1.0, and preferably 0.4 to 0.7 and most preferably 0.6 to 0.8.

According to an embodiment, the degree of substitution of the cinnamaldehyde group on the carboxylated polysaccharide may be from about 0.01 to about 0.20, or from about 0.02 to about 0.20, or from about 0.03 to about 0.20, or from about 0.04 to about 0.20, or from about 0.05 to about 0.20, or from about 0.06 to about 0.20, or from about 0.07 to about 0.20, or from about 0.08 to about 0.20, or from about 0.09 to about 0.20, or from about 0.10 to about 0.20, or from about 0.11 to about 0.20, or from about 0.12 to about 0.20, or from about 0.13 to about 0.20, or from about 0.14 to about 0.20, or from about 0.15 to about 0.20, or from about 0.16 to about 0.20, or from about 0.17 to about 0.20, or from about 0.18 to about 0.20, or from about 0.19 to about 0.20, preferably from 0.01 to 0.1. This corresponds to one to five molecules of cinnamaldehyde for every 100 sugar.

The following are embodiments, groups and substituents of the compounds according to Formula (I) or (Ia) or (Ib), which are described hereinafter in detail.

n:

In one subset of compounds of Formula (I), n is 1.

m:

In one subset of compounds of Formula (I), m is 1.

Any and each individual definition of n as set out herein may be combined with any and each individual definition of R¹, R^{1′}, R², R³, and R⁴ as set out herein.

In embodiments of the present invention, the carboxylated polysaccharide is of general formula (I), or pharmaceutically acceptable salts thereof, and stereoisomers thereof:

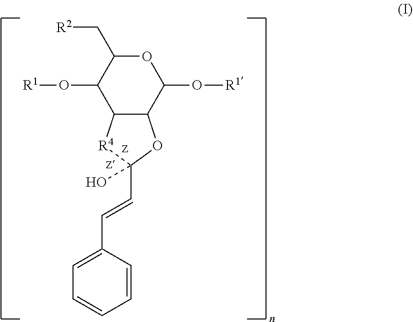

wherein
n=15−1.67×10⁵
R¹ and R^{1′} are each independently

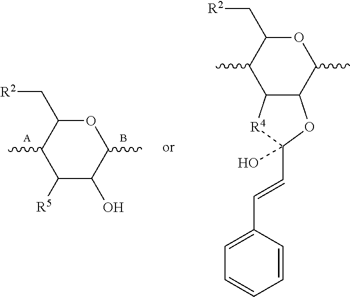

R² is OH, R¹, O—(CH₂)ₘCO₂R³, O—(CH₂)ₘ(CO₂R³)(CH₂)ₚCH₃, O—CO(CH₂)ₘ(CO₂R³)(CH₂)ₚCh₃, O—CO(CH₂)ₘCO₂R³, O—CO—CH₂—CH—COR³, or O—CO—CH₂—CH(CH₂)ₚ—COR³;
R³ is absent or H;
R⁴ is O when bond Z is present and R⁵ when bond Z is absent;
bond Z′ is present when bond Z is absent;
R⁵ is OH or R¹;
m=0-4;
p=0-8, and
[symbol] indicates a covalent bond connecting to the R^{1′}, and [symbol] indicates a covalent bond connecting to the R¹.

According to another embodiment, the carboxylated polysaccharide of general formula (I), is of general formula (Ia):

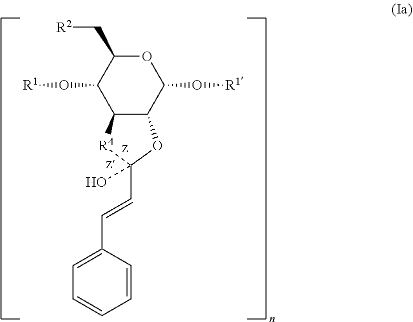

wherein
n=15−1.67×10⁵;
R¹ and R^{1′} are each independently

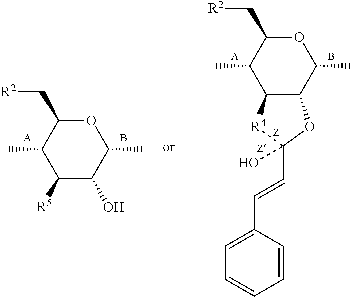

R² is OH, R¹, O—(CH₂)ₘCO₂R⁴, O—(CH₂)ₘ(CO₂R³)(CH₂)ₚCH₃, O—CO(CH₂)ₘ(CO₂R³)(CH₂)ₚCH₃, O—CO(CH₂)ₘCO₂R³, O—CO—CH₂—CH—COR³, or O—CO—CH₂—CH(CH₂)ₚ—COR³;
R³ is absent or H;
m=0-4;
p=0-8;
R⁴ is O when bond Z is present and R⁵ when bond Z is absent;
bond Z′ is present when bond Z is absent;
R⁵ is OH or R¹; and
[symbol] indicates a covalent bond connecting to the R^{1′}, and [symbol] indicates a covalent bond connecting to the R¹.

According to another embodiment, the carboxylated polysaccharide of general formula (I), is of general formula (Ib):

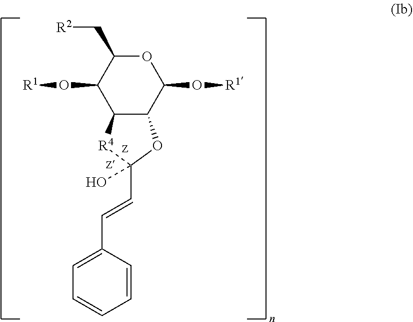

wherein
n=15−1.67×10⁵;
R¹ and R^{1′} are each independently

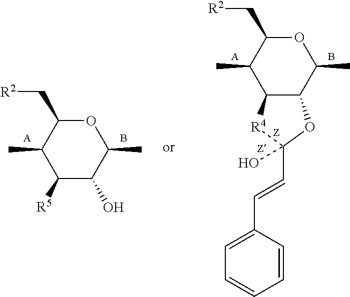

R² is OH, R¹, O—(CH₂)ₘCO₂R⁴, O—(CH₂)ₘ(CO₂R³)(CH₂)ₚCH₃, O—CO(CH₂)ₘ(CO₂R³)(CH₂)ₚCH₃, O—CO(CH₂)ₘCO₂R³, O—CO—CH₂—CH—COR³, or O—CO—CH₂—CH(CH₂)ₚ—COR³;
R³ is absent or H;
m=0-4;
p=0-8;
R⁴ is O when bond Z is present and R⁵ when bond Z is absent;
bond Z′ is present when bond Z is absent;
R⁵ is OH or R¹; and
bond connecting to the R^{1′}, and [symbol] indicates a covalent bond connecting to the R¹.

In embodiments, the R² is O—CH₂COO⁻.

In embodiment, the number of repeating units “n” above mentioned is indicated for carboxylated polysaccharides having a minimum of about 50 individual sugar units to a maximum of about 500,000 sugar units. This represents approximately minimum chain length of about 10⁴ g/mol to maximum chain length of about 10⁸ g/mol.

In embodiments, the carboxylated polysaccharides may be branched carboxylated polysaccharides as well as non-branched polysaccharides. For example, the carboxylated polysaccharides may be carboxylated starches including amylose and amylopectine, and carboxylated celluloses including non-branched celluloses, and hemicelluloses (branched).

### Artesunate Water Soluble Complex

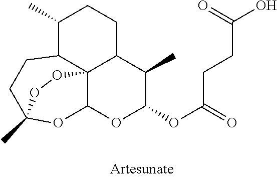 [SMILES: C[C@@H]1CCC2[C@@H](C)[C@H](OC(=O)CCC(=O)O)OC3O[C@@]4(C)CCC1[C@]32OO4]

Artesunate is a hemi-succinate derivative of artemisinin. Artesunate is unstable in aqueous, acidic and basic conditions, and is sensitive to light. Also, salt forms of artesunate (such as sodium artesunate) are sticking, have low flowability, and are difficult to handle.

Commercially artesunate is available in dry powder form of artesunic acid which is poorly soluble in aqueous medium. According to the DrugBank, the water solubility of artesunic acid is about of 0.678 mg/mL (or 0.68%, w/w). Because of this poor solubility, commercially available injection of artesunate (60 mg) requires 1 mL of sodium bicarbonate (5% w/w) solution and dilution in 5 mL of saline (0.9% NaCl w/w) solution immediately before use. This mode of administration is inconvenient, prone to error and could be improved. After parenteral administration, it is rapidly hydrolyzed to the active metabolite dihydroartemisinin (DHA).

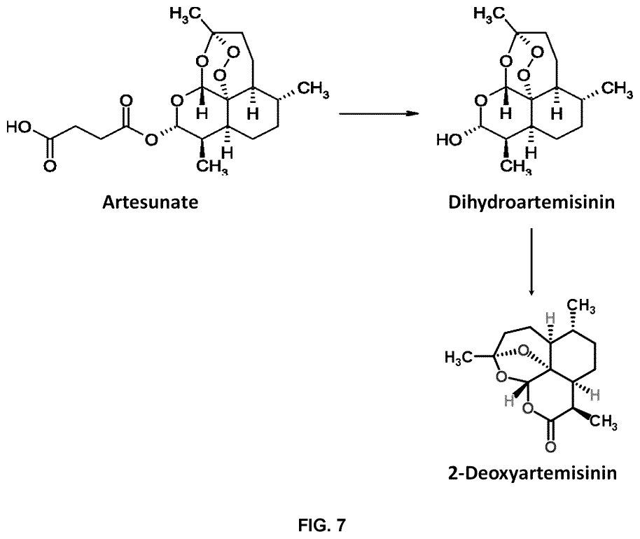

For oral administration, artesunate generally remains insoluble in acid gastric and is rapidly hydrolyzed in stomach and its rate of conversion in DHA is pH dependent. Furthermore, the hydrolysis of artesunate to DHA is carried out during the stomach transit before entering the systemic circulation. Clinically, artesunate serves essentially as a prodrug for DHA. Of the current clinically used artemisinin derivatives, DHA elicits the highest neurotoxicity in cellular and animal assay. In addition, each of artesunate and DHA decomposes readily under aqueous acidic conditions to provide the inert end product 2-deoxyartemisinin. It is important to mention that only molecules with a conserved endoperoxide bridge have antimalarial activity. The other non-peroxide-contaning degradation products, including 2-deoxyartemisinin, do not. FIG. 7 shows the acid-catalysed hydrolysis of artesunate to DHA and then to 2-deoxyartemisinin during gastric transit.

In this context, there is provided a preparation of water soluble artesunate in comparison to artesunate under salt forms and artesunate entrapped in starch glycolate known in the art (e.g. WO 2006/049391 and WO 2015/127537 respectively). This water-soluble artesunate (WSA) powder is unexpectedly stable under salt form and more soluble, not only in aqueous medium, but also in gastric acid or intestinal fluids at various temperatures. Unexpectedly, no precipitate is observed after 24 h in solution compared to commercial artesunate. This solubility in gastric acid environment is an important feature suitable to formulate the extended release tablet by floating or swelling.

According to another embodiment, there is provided an extended release gastro retentive dosage form comprising:
an artesunate emulsion having a pH value of from about 7.5 to 7.9 and comprising an artesunate or pharmaceutically acceptable salts thereof, and stereoisomers thereof stabilized with an emulsifying agent.

In embodiments, the weight ratio of the artesunate pharmaceutically acceptable salt and the emulsifying agent in the artesunate emulsion may be from about 9:1 to about 1:1, or from about 8:1 to about 1:1, or from about 7:1 to about 1:1, or from about 6:1 to about 1:1, or from about 5:1 to about 1:1, or from about 4:1 to about 1:1, or from about 3:1 to about 1:1, or from about 2:1 to about 1:1, or from about 9:2 to about 1:1, or from about 7:2 to about 1:1, or from about 5:2 to about 1:1, or from about 3:2 to about 1:1, or from about 1:2 to about 1:1, or from about 9:3 to about 1:1, or from about 8:3 to about 1:1, or from about 7:3 to about 1:1, or from about 5:3 to about 1:1, or from about 4:3 to about 1:1, or from about 2:3 to about 1:1, or from about 1:3 to about 1:1, or from about 9:4 to about 1:1, or from about 7:4 to about 1:1, or from about 5:4 to about 1:1, or from about 3:4 to about 1:1, or from about 1:4 to about 1:1, or from about 9:5 to about 1:1, or from about 8:5 to about 1:1, or from about 7:5 to about 1:1, or from about 6:5 to about 1:1, or from about 4:5 to about 1:1, or from about 3:5 to about 1:1, or from about 2:5 to about 1:1, or from about 1:5 to about 1:1, or from about 7:6 to about 1:1, or from about 5:6 to about 1:1, or from about 1:6 to about 1:1, or from about 9:7 to about 1:1, or from about 8:7 to about 1:1, or from about 6:7 to about 1:1, or from about 5:7 to about 1:1, or from about 4:7 to about 1:1, or from about 3:7 to about 1:1, or from about 2:7 to about 1:1, or from about 1:7 to about 1:1, or from about 9:8 to about 1:1, or from about 7:8 to about 1:1, or from about 5:8 to about 1:1, or from about 3:8 to about 1:1, or from about 1:8 to about 1:1, or from about 8:9 to about 1:1, or from about 7:9 to about 1:1, or from about 5:9 to about 1:1, or from about 4:9 to about 1:1, or from about 2:9 to about 1:1, or from about 1:9 to about 1:1, or from about 9:1 to about 1:2, or from about 8:1 to about 1:2, or from about 7:1 to about 1:2, or from about 6:1 to about 1:2, or from about 5:1 to about 1:2, or from about 4:1 to about 1:2, or from about 3:1 to about 1:2, or from about 2:1 to about 1:2, or from about 9:2 to about 1:2, or from about 7:2 to about 1:2, or from about 5:2 to about 1:2, or from about 3:2 to about 1:2, or from about 1:2 to about 1:2, or from about 9:3 to about 1:2, or from about 8:3 to about 1:2, or from about 7:3 to about 1:2, or from about 5:3 to about 1:2, or from about 4:3 to about 1:2, or from about 2:3 to about 1:2, or from about 1:3 to about 1:2, or from about 9:4 to about 1:2, or from about 7:4 to about 1:2, or from about 5:4 to about 1:2, or from about 3:4 to about 1:2, or from about 1:4 to about 1:2, or from about 9:5 to about 1:2, or from about 8:5 to about 1:2, or from about 7:5 to about 1:2, or from about 6:5 to about 1:2, or from about 4:5 to about 1:2, or from about 3:5 to about 1:2, or from about 2:5 to about 1:2, or from about 1:5 to about 1:2, or from about 7:6 to about 1:2, or from about 5:6 to about 1:2, or from about 1:6 to about 1:2, or from about 9:7 to about 1:2, or from about 8:7 to about 1:2, or from about 6:7 to about 1:2, or from about 5:7 to about 1:2, or from about 4:7 to about 1:2, or from about 3:7 to about 1:2, or from about 2:7 to about 1:2, or from about 1:7 to about 1:2, or from about 9:8 to about 1:2, or from about 7:8 to about 1:2, or from about 5:8 to about 1:2, or from about 3:8 to about 1:2, or from about 1:8 to about 1:2, or from about 8:9 to about 1:2, or from about 7:9 to about 1:2, or from about 5:9 to about 1:2, or from about 4:9 to about 1:2, or from about 2:9 to about 1:2, or from about 1:9 to about 1:2, or from about 9:1 to about 1:3, or from about 8:1 to about 1:3, or from about 7:1 to about 1:3, or from about 6:1 to about 1:3, or from about 5:1 to about 1:3, or from about 4:1 to about 1:3, or from about 3:1 to about 1:3, or from about 2:1 to about 1:3, or from about 9:2 to about 1:3, or from about 7:2 to about 1:3, or from about 5:2 to about 1:3, or from about 3:2 to about 1:3, or from about 1:3 to about 1:3, or from about 9:3 to about 1:3, or from about 8:3 to about 1:3, or from about 7:3 to about 1:3, or from about 5:3 to about 1:3, or from about 4:3 to about 1:3, or from about 2:3 to about 1:3, or from about 9:4 to about 1:3, or from about 7:4 to about 1:3, or from about 5:4 to about 1:3, or from about 3:4 to about 1:3, or from about 1:4 to about 1:3, or from about 9:5 to about 1:3, or from about 8:5 to about 1:3, or from about 7:5 to about 1:3, or from about 6:5 to about 1:3, or from about 4:5 to about 1:3, or from about 3:5 to about 1:3, or from about 2:5 to about 1:3, or from about 1:5 to about 1:3, or from about 7:6 to about 1:3, or from about 5:6 to about 1:3, or from about 1:6 to about 1:3, or from about 9:7 to about 1:3, or from about 8:7 to about 1:3, or from about 6:7 to about 1:3, or from about 5:7 to about 1:3, or from about 4:7 to about 1:3, or from about 3:7 to about 1:3, or from about 2:7 to about 1:3, or from about 1:7 to about 1:3, or from about 9:8 to about 1:3, or from about 7:8 to about 1:3, or from about 5:8 to about 1:3, or from about 3:8 to about 1:3, or from about 1:8 to about 1:3, or from about 8:9 to about 1:3, or from about 7:9 to about 1:3, or from about 5:9 to about 1:3, or from about 4:9 to about 1:3, or from about 2:9 to about 1:3, or from about 1:9 to about 1:3, or from about 9:1 to about 1:4, or from about 8:1 to about 1:4, or from about 7:1 to about 1:4, or from about 6:1 to about 1:4, or from about 5:1 to about 1:4, or from about 4:1 to about 1:4, or from about 3:1 to about 1:4, or from about 2:1 to about 1:4, or from about 9:2 to about 1:4, or from about 7:2 to about 1:4, or from about 5:2 to about 1:4, or from about 3:2 to about 1:4, or from about 1:2 to about 1:4, or from about 9:3 to about 1:4, or from about 8:3 to about 1:4, or from about 7:3 to about 1:4, or from about 5:3 to about 1:4, or from about 4:3 to about 1:4, or from about 2:3 to about 1:4, or from about 1:3 to about 1:4, or from about 9:4 to about 1:4, or from about 7:4 to about 1:4, or from about 5:4 to about 1:4, or from about 3:4 to about 1:4, or from about 1:4 to about 1:4, or from about 9:5 to about 1:4, or from about 8:5 to about 1:4, or from about 7:5 to about 1:4, or from about 6:5 to about 1:4, or from about 4:5 to about 1:4, or from about 3:5 to about 1:4, or from about 2:5 to about 1:4, or from about 1:5 to about 1:4, or from about 7:6 to about 1:4, or from about 5:6 to about 1:4, or from about 1:6 to about 1:4, or from about 9:7 to about 1:4, or from about 8:7 to about 1:4, or from about 6:7 to about 1:4, or from about 5:7 to about 1:4, or from about 4:7 to about 1:4, or from about 3:7 to about 1:4, or from about 2:7 to about 1:4, or from about 1:7 to about 1:4, or from about 9:8 to about 1:4, or from about 7:8 to about 1:4, or from about 5:8 to about 1:4, or from about 3:8 to about 1:4, or from about 1:8 to about 1:4, or from about 8:9 to about 1:4, or from about 7:9 to about 1:4, or from about 5:9 to about 1:4, or from about 4:9 to about 1:4, or from about 2:9 to about 1:4, or from about 1:9 to about 1:4, or from about 9:1 to about 1:5, or from about 8:1 to about 1:5, or from about 7:1 to about 1:5, or from about 6:1 to about 1:5, or from about 5:1 to about 1:5, or from about 4:1 to about 1:5, or from about 3:1 to about 1:5, or from about 2:1 to about 1:5, or from about 1:5 to about 1:5, or from about 9:2 to about 1:5, or from about 7:2 to about 1:5, or from about 5:2 to about 1:5, or from about 3:2 to about 1:5, or from about 1:2 to about 1:5, or from about 9:3 to about 1:5, or from about 8:3 to about 1:5, or from about 7:3 to about 1:5, or from about 5:3 to about 1:5, or from about 4:3 to about 1:5, or from about 2:3 to about 1:5, or from about 1:3 to about 1:5, or from about 9:4 to about 1:5, or from about 7:4 to about 1:5, or from about 5:4 to about 1:5, or from about 3:4 to about 1:5, or from about 1:4 to about 1:5, or from about 9:5 to about 1:5, or from about 8:5 to about 1:5, or from about 7:5 to about 1:5, or from about 6:5 to about 1:5, or from about 4:5 to about 1:5, or from about 3:5 to about 1:5, or from about 2:5 to about 1:5, or from about 7:6 to about 1:5, or from about 5:6 to about 1:5, or from about 1:6 to about 1:5, or from about 9:7 to about 1:5, or from about 8:7 to about 1:5, or from about 6:7 to about 1:5, or from about 5:7 to about 1:5, or from about 4:7 to about 1:5, or from about 3:7 to about 1:5, or from about 2:7 to about 1:5, or from about 1:7 to about 1:5, or from about 9:8 to about 1:5, or from about 7:8 to about 1:5, or from about 5:8 to about 1:5, or from about 3:8 to about 1:5, or from about 1:8 to about 1:5, or from about 8:9 to about 1:5, or from about 7:9 to about 1:5, or from about 5:9 to about 1:5, or from about 4:9 to about 1:5, or from about 2:9 to about 1:5, or from about 1:9 to about 1:5, or from about 9:1 to about 2:1, or from about 8:1 to about 2:1, or from about 7:1 to about 2:1, or from about 6:1 to about 2:1, or from about 5:1 to about 2:1, or from about 4:1 to about 2:1, or from about 3:1 to about 2:1, or from about 2:1 to about 2:1, or from about 9:2 to about 2:1, or from about 7:2 to about 2:1, or from about 5:2 to about 2:1, or from about 3:2 to about 2:1, or from about 1:2 to about 2:1, or from about 9:3 to about 2:1, or from about 8:3 to about 2:1, or from about 7:3 to about 2:1, or from about 5:3 to about 2:1, or from about 4:3 to about 2:1, or from about 2:3 to about 2:1, or from about 1:3 to about 2:1, or from about 9:4 to about 2:1, or from about 7:4 to about 2:1, or from about 5:4 to about 2:1, or from about 3:4 to about 2:1, or from about 1:4 to about 2:1, or from about 9:5 to about 2:1, or from about 8:5 to about 2:1, or from about 7:5 to about 2:1, or from about 6:5 to about 2:1, or from about 4:5 to about 2:1, or from about 3:5 to about 2:1, or from about 2:5 to about 2:1, or from about 1:5 to about 2:1, or from about 7:6 to about 2:1, or from about 5:6 to about 2:1, or from about 1:6 to about 2:1, or from about 9:7 to about 2:1, or from about 8:7 to about 2:1, or from about 6:7 to about 2:1, or from about 5:7 to about 2:1, or from about 4:7 to about 2:1, or from about 3:7 to about 2:1, or from about 2:7 to about 2:1, or from about 1:7 to about 2:1, or from about 9:8 to about 2:1, or from about 7:8 to about 2:1, or from about 5:8 to about 2:1, or from about 3:8 to about 2:1, or from about 1:8 to about 2:1, or from about 8:9 to about 2:1, or from about 7:9 to about 2:1, or from about 5:9 to about 2:1, or from about 4:9 to about 2:1, or from about 2:9 to about 2:1, or from about 1:9 to about 2:1, or from about 9:1 to about 2:3, or from about 8:1 to about 2:3, or from about 7:1 to about 2:3, or from about 6:1 to about 2:3, or from about 5:1 to about 2:3, or from about 4:1 to about 2:3, or from about 2:3 to about 2:3, or from about 2:1 to about 2:3, or from about 9:2 to about 2:3, or from about 7:2 to about 2:3, or from about 5:2 to about 2:3, or from about 3:2 to about 2:3, or from about 2:3 to about 2:3, or from about 9:3 to about 2:3, or from about 8:3 to about 2:3, or from about 7:3 to about 2:3, or from about 5:3 to about 2:3, or from about 4:3 to about 2:3, or from about 2:3 to about 2:3, or from about 1:3 to about 2:3, or from about 9:4 to about 2:3, or from about 7:4 to about 2:3, or from about 5:4 to about 2:3, or from about 3:4 to about 2:3, or from about 1:4 to about 2:3, or from about 9:5 to about 2:3, or from about 8:5 to about 2:3, or from about 7:5 to about 2:3, or from about 6:5 to about 2:3, or from about 4:5 to about 2:3, or from about 3:5 to about 2:3, or from about 2:5 to about 2:3, or from about 1:5 to about 2:3, or from about 7:6 to about 2:3, or from about 5:6 to about 2:3, or from about 1:6 to about 2:3, or from about 9:7 to about 2:3, or from about 8:7 to about 2:3, or from about 6:7 to about 2:3, or from about 5:7 to about 2:3, or from about 4:7 to about 2:3, or from about 3:7 to about 2:3, or from about 2:7 to about 2:3, or from about 1:7 to about 2:3, or from about 9:8 to about 2:3, or from about 7:8 to about 2:3, or from about 5:8 to about 2:3, or from about 3:8 to about 2:3, or from about 1:8 to about 2:3, or from about 8:9 to about 2:3, or from about 7:9 to about 2:3, or from about 5:9 to about 2:3, or from about 4:9 to about 2:3, or from about 2:9 to about 2:3, or from about 1:9 to about 2:3, or from about 9:1 to about 2:5, or from about 8:1 to about 2:5, or from about 7:1 to about 2:5, or from about 6:1 to about 2:5, or from about 5:1 to about 2:5, or from about 4:1 to about 2:5, or from about 3:1 to about 2:5, or from about 2:1 to about 2:5, or from about 9:2 to about 2:5, or from about 7:2 to about 2:5, or from about 5:2 to about 2:5, or from about 3:2 to about 2:5, or from about 2:5 to about 2:5, or from about 9:3 to about 2:5, or from about 8:3 to about 2:5, or from about 7:3 to about 2:5, or from about 5:3 to about 2:5, or from about 4:3 to about 2:5, or from about 2:3 to about 2:5, or from about 9:4 to about 2:5, or from about 7:4 to about 2:5, or from about 5:4 to about 2:5, or from about 3:4 to about 2:5, or from about 1:4 to about 2:5, or from about 9:5 to about 2:5, or from about 8:5 to about 2:5, or from about 7:5 to about 2:5, or from about 6:5 to about 2:5, or from about 4:5 to about 2:5, or from about 3:5 to about 2:5, or from about 2:5 to about 2:5, or from about 1:5 to about 2:5, or from about 7:6 to about 2:5, or from about 5:6 to about 2:5, or from about 1:6 to about 2:5, or from about 9:7 to about 2:5, or from about 8:7 to about 2:5, or from about 6:7 to about 2:5, or from about 5:7 to about 2:5, or from about 4:7 to about 2:5, or from about 3:7 to about 2:5, or from about 2:7 to about 2:5, or from about 1:7 to about 2:5, or from about 9:8 to about 2:5, or from about 7:8 to about 2:5, or from about 5:8 to about 2:5, or from about 3:8 to about 2:5, or from about 1:8 to about 2:5, or from about 8:9 to about 2:5, or from about 7:9 to about 2:5, or from about 5:9 to about 2:5, or from about 4:9 to about 2:5, or from about 2:9 to about 2:5, or from about 1:9 to about 2:5, or from about 9:1 to about 3:1, or from about 8:1 to about 3:1, or from about 7:1 to about 3:1, or from about 6:1 to about 3:1, or from about 5:1 to about 3:1, or from about 4:1 to about 3:1, or from about 3:1 to about 3:1, or from about 2:1 to about 3:1, or from about 9:2 to about 3:1, or from about 7:2 to about 3:1, or from about 5:2 to about 3:1, or from about 3:2 to about 3:1, or from about 1:2 to about 3:1, or from about 9:3 to about 3:1, or from about 8:3 to about 3:1, or from about 7:3 to about 3:1, or from about 5:3 to about 3:1, or from about 4:3 to about 3:1, or from about 2:3 to about 3:1, or from about 1:3 to about 3:1, or from about 9:4 to about 3:1, or from about 7:4 to about 3:1, or from about 5:4 to about 3:1, or from about 3:4 to about 3:1, or from about 1:4 to about 3:1, or from about 9:5 to about 3:1, or from about 8:5 to about 3:1, or from about 7:5 to about 3:1, or from about 6:5 to about 3:1, or from about 4:5 to about 3:1, or from about 3:5 to about 3:1, or from about 2:5 to about 3:1, or from about 1:5 to about 3:1, or from about 7:6 to about 3:1, or from about 5:6 to about 3:1, or from about 1:6 to about 3:1, or from about 9:7 to about 3:1, or from about 8:7 to about 3:1, or from about 6:7 to about 3:1, or from about 5:7 to about 3:1, or from about 4:7 to about 3:1, or from about 3:7 to about 3:1, or from about 2:7 to about 3:1, or from about 1:7 to about 3:1, or from about 9:8 to about 3:1, or from about 7:8 to about 3:1, or from about 5:8 to about 3:1, or from about 3:8 to about 3:1, or from about 1:8 to about 3:1, or from about 8:9 to about 3:1, or from about 7:9 to about 3:1, or from about 5:9 to about 3:1, or from about 4:9 to about 3:1, or from about 2:9 to about 3:1, or from about 1:9 to about 3:1, or from about 9:1 to about 3:2, or from about 8:1 to about 3:2, or from about 7:1 to about 3:2, or from about 6:1 to about 3:2, or from about 5:1 to about 3:2, or from about 4:1 to about 3:2, or from about 3:1 to about 3:2, or from about 2:1 to about 3:2, or from about 9:2 to about 3:2, or from about 7:2 to about 3:2, or from about 5:2 to about 3:2, or from about 3:2 to about 3:2, or from about 3:2 to about 3:2, or from about 9:3 to about 3:2, or from about 8:3 to about 3:2, or from about 7:3 to about 3:2, or from about 5:3 to about 3:2, or from about 4:3 to about 3:2, or from about 2:3 to about 3:2, or from about 1:3 to about 3:2, or from about 9:4 to about 3:2, or from about 7:4 to about 3:2, or from about 5:4 to about 3:2, or from about 3:4 to about 3:2, or from about 1:4 to about 3:2, or from about 9:5 to about 3:2, or from about 8:5 to about 3:2, or from about 7:5 to about 3:2, or from about 6:5 to about 3:2, or from about 4:5 to about 3:2, or from about 3:5 to about 3:2, or from about 2:5 to about 3:2, or from about 1:5 to about 3:2, or from about 7:6 to about 3:2, or from about 5:6 to about 3:2, or from about 1:6 to about 3:2, or from about 9:7 to about 3:2, or from about 8:7 to about 3:2, or from about 6:7 to about 3:2, or from about 5:7 to about 3:2, or from about 4:7 to about 3:2, or from about 3:7 to about 3:2, or from about 2:7 to about 3:2, or from about 1:7 to about 3:2, or from about 9:8 to about 3:2, or from about 7:8 to about 3:2, or from about 5:8 to about 3:2, or from about 3:8 to about 3:2, or from about 1:8 to about 3:2, or from about 8:9 to about 3:2, or from about 7:9 to about 3:2, or from about 5:9 to about 3:2, or from about 4:9 to about 3:2, or from about 2:9 to about 3:2, or from about 1:9 to about 3:2, or from about 9:1 to about 3:4, or from about 8:1 to about 3:4, or from about 7:1 to about 3:4, or from about 6:1 to about 3:4, or from about 5:1 to about 3:4, or from about 4:1 to about 3:4, or from about 3:1 to about 3:4, or from about 2:1 to about 3:4, or from about 9:2 to about 3:4, or from about 7:2 to about 3:4, or from about 5:2 to about 3:4, or from about 3:2 to about 3:4, or from about 3:4 to about 3:4, or from about 9:3 to about 3:4, or from about 8:3 to about 3:4, or from about 7:3 to about 3:4, or from about 5:3 to about 3:4, or from about 4:3 to about 3:4, or from about 2:3 to about 3:4, or from about 9:4 to about 3:4, or from about 7:4 to about 3:4, or from about 5:4 to about 3:4, or from about 3:4 to about 3:4, or from about 1:4 to about 3:4, or from about 9:5 to about 3:4, or from about 8:5 to about 3:4, or from about 7:5 to about 3:4, or from about 6:5 to about 3:4, or from about 4:5 to about 3:4, or from about 3:5 to about 3:4, or from about 2:5 to about 3:4, or from about 1:5 to about 3:4, or from about 7:6 to about 3:4, or from about 5:6 to about 3:4, or from about 1:6 to about 3:4, or from about 9:7 to about 3:4, or from about 8:7 to about 3:4, or from about 6:7 to about 3:4, or from about 5:7 to about 3:4, or from about 4:7 to about 3:4, or from about 3:7 to about 3:4, or from about 2:7 to about 3:4, or from about 1:7 to about 3:4, or from about 9:8 to about 3:4, or from about 7:8 to about 3:4, or from about 5:8 to about 3:4, or from about 3:8 to about 3:4, or from about 1:8 to about 3:4, or from about 8:9 to about 3:4, or from about 7:9 to about 3:4, or from about 5:9 to about 3:4, or from about 4:9 to about 3:4, or from about 2:9 to about 3:4, or from about 1:9 to about 3:4, or from about 9:1 to about 3:5, or from about 8:1 to about 3:5, or from about 7:1 to about 3:5, or from about 6:1 to about 3:5, or from about 5:1 to about 3:5, or from about 4:1 to about 3:5, or from about 3:1 to about 3:5, or from about 2:1 to about 3:5, or from about 9:2 to about 3:5, or from about 7:2 to about 3:5, or from about 5:2 to about 3:5, or from about 3:2 to about 3:5, or from about 1:2 to about 3:5, or from about 9:3 to about 3:5, or from about 8:3 to about 3:5, or from about 7:3 to about 3:5, or from about 5:3 to about 3:5, or from about 4:3 to about 3:5, or from about 2:3 to about 3:5, or from about 1:3 to about 3:5, or from about 9:4 to about 3:5, or from about 7:4 to about 3:5, or from about 5:4 to about 3:5, or from about 3:4 to about 3:5, or from about 3:5 to about 3:5, or from about 9:5 to about 3:5, or from about 8:5 to about 3:5, or from about 7:5 to about 3:5, or from about 6:5 to about 3:5, or from about 4:5 to about 3:5, or from about 3:5 to about 3:5, or from about 2:5 to about 3:5, or from about 1:5 to about 3:5, or from about 7:6 to about 3:5, or from about 5:6 to about 3:5, or from about 1:6 to about 3:5, or from about 9:7 to about 3:5, or from about 8:7 to about 3:5, or from about 6:7 to about 3:5, or from about 5:7 to about 3:5, or from about 4:7 to about 3:5, or from about 3:7 to about 3:5, or from about 2:7 to about 3:5, or from about 1:7 to about 3:5, or from about 9:8 to about 3:5, or from about 7:8 to about 3:5, or from about 5:8 to about 3:5, or from about 3:8 to about 3:5, or from about 1:8 to about 3:5, or from about 8:9 to about 3:5, or from about 7:9 to about 3:5, or from about 5:9 to about 3:5, or from about 4:9 to about 3:5, or from about 2:9 to about 3:5, or from about 1:9 to about 3:5, or from about 9:1 to about 4:1, or from about 8:1 to about 4:1, or from about 7:1 to about 4:1, or from about 6:1 to about 4:1, or from about 5:1 to about 4:1, or from about 4:1 to about 4:1, or from about 3:1 to about 4:1, or from about 2:1 to about 4:1, or from about 9:2 to about 4:1, or from about 7:2 to about 4:1, or from about 5:2 to about 4:1, or from about 3:2 to about 4:1, or from about 1:2 to about 4:1, or from about 9:3 to about 4:1, or from about 8:3 to about 4:1, or from about 7:3 to about 4:1, or from about 5:3 to about 4:1, or from about 4:3 to about 4:1, or from about 2:3 to about 4:1, or from about 1:3 to about 4:1, or from about 9:4 to about 4:1, or from about 7:4 to about 4:1, or from about 5:4 to about 4:1, or from about 3:4 to about 4:1, or from about 1:4 to about 4:1, or from about 9:5 to about 4:1, or from about 8:5 to about 4:1, or from about 7:5 to about 4:1, or from about 6:5 to about 4:1, or from about 4:5 to about 4:1, or from about 3:5 to about 4:1, or from about 2:5 to about 4:1, or from about 1:5 to about 4:1, or from about 7:6 to about 4:1, or from about 5:6 to about 4:1, or from about 1:6 to about 4:1, or from about 9:7 to about 4:1, or from about 8:7 to about 4:1, or from about 6:7 to about 4:1, or from about 5:7 to about 4:1, or from about 4:7 to about 4:1, or from about 3:7 to about 4:1, or from about 2:7 to about 4:1, or from about 1:7 to about 4:1, or from about 9:8 to about 4:1, or from about 7:8 to about 4:1, or from about 5:8 to about 4:1, or from about 3:8 to about 4:1, or from about 1:8 to about 4:1, or from about 8:9 to about 4:1, or from about 7:9 to about 4:1, or from about 5:9 to about 4:1, or from about 4:9 to about 4:1, or from about 2:9 to about 4:1, or from about 1:9 to about 4:1, or from about 9:1 to about 4:3, or from about 8:1 to about 4:3, or from about 7:1 to about 4:3, or from about 6:1 to about 4:3, or from about 5:1 to about 4:3, or from about 4:1 to about 4:3, or from about 3:1 to about 4:3, or from about 2:1 to about 4:3, or from about 9:2 to about 4:3, or from about 7:2 to about 4:3, or from about 5:2 to about 4:3, or from about 3:2 to about 4:3, or from about 4:3 to about 4:3, or from about 9:3 to about 4:3, or from about 8:3 to about 4:3, or from about 7:3 to about 4:3, or from about 5:3 to about 4:3, or from about 4:3 to about 4:3, or from about 2:3 to about 4:3, or from about 1:3 to about 4:3, or from about 9:4 to about 4:3, or from about 7:4 to about 4:3, or from about 5:4 to about 4:3, or from about 3:4 to about 4:3, or from about 1:4 to about 4:3, or from about 9:5 to about 4:3, or from about 8:5 to about 4:3, or from about 7:5 to about 4:3, or from about 6:5 to about 4:3, or from about 4:5 to about 4:3, or from about 3:5 to about 4:3, or from about 2:5 to about 4:3, or from about 1:5 to about 4:3, or from about 7:6 to about 4:3, or from about 5:6 to about 4:3, or from about 1:6 to about 4:3, or from about 9:7 to about 4:3, or from about 8:7 to about 4:3, or from about 6:7 to about 4:3, or from about 5:7 to about 4:3, or from about 4:7 to about 4:3, or from about 3:7 to about 4:3, or from about 2:7 to about 4:3, or from about 1:7 to about 4:3, or from about 9:8 to about 4:3, or from about 7:8 to about 4:3, or from about 5:8 to about 4:3, or from about 3:8 to about 4:3, or from about 1:8 to about 4:3, or from about 8:9 to about 4:3, or from about 7:9 to about 4:3, or from about 5:9 to about 4:3, or from about 4:9 to about 4:3, or from about 2:9 to about 4:3, or from about 1:9 to about 4:3, or from about 9:1 to about 4:5, or from about 8:1 to about 4:5, or from about 7:1 to about 4:5, or from about 6:1 to about 4:5, or from about 5:1 to about 4:5, or from about 4:1 to about 4:5, or from about 3:1 to about 4:5, or from about 2:1 to about 4:5, or from about 9:2 to about 4:5, or from about 7:2 to about 4:5, or from about 5:2 to about 4:5, or from about 3:2 to about 4:5, or from about 4:5 to about 4:5, or from about 9:3 to about 4:5, or from about 8:3 to about 4:5, or from about 7:3 to about 4:5, or from about 5:3 to about 4:5, or from about 4:3 to about 4:5, or from about 2:3 to about 4:5, or from about 9:4 to about 4:5, or from about 7:4 to about 4:5, or from about 5:4 to about 4:5, or from about 3:4 to about 4:5, or from about 1:4 to about 4:5, or from about 9:5 to about 4:5, or from about 8:5 to about 4:5, or from about 7:5 to about 4:5, or from about 6:5 to about 4:5, or from about 4:5 to about 4:5, or from about 3:5 to about 4:5, or from about 2:5 to about 4:5, or from about 1:5 to about 4:5, or from about 7:6 to about 4:5, or from about 5:6 to about 4:5, or from about 1:6 to about 4:5, or from about 9:7 to about 4:5, or from about 8:7 to about 4:5, or from about 6:7 to about 4:5, or from about 5:7 to about 4:5, or from about 4:7 to about 4:5, or from about 3:7 to about 4:5, or from about 2:7 to about 4:5, or from about 1:7 to about 4:5, or from about 9:8 to about 4:5, or from about 7:8 to about 4:5, or from about 5:8 to about 4:5, or from about 3:8 to about 4:5, or from about 1:8 to about 4:5, or from about 8:9 to about 4:5, or from about 7:9 to about 4:5, or from about 5:9 to about 4:5, or from about 4:9 to about 4:5, or from about 2:9 to about 4:5, or from about 1:9 to about 4:5, or from about 9:1 to about 5:1, or from about 8:1 to about 5:1, or from about 7:1 to about 5:1, or from about 6:1 to about 5:1, or from about 5:1 to about 5:1, or from about 4:1 to about 5:1, or from about 3:1 to about 5:1, or from about 2:1 to about 5:1, or from about 9:2 to about 5:1, or from about 7:2 to about 5:1, or from about 5:2 to about 5:1, or from about 3:2 to about 5:1, or from about 1:2 to about 5:1, or from about 9:3 to about 5:1, or from about 8:3 to about 5:1, or from about 7:3 to about 5:1, or from about 5:3 to about 5:1, or from about 4:3 to about 5:1, or from about 2:3 to about 5:1, or from about 1:3 to about 5:1, or from about 9:4 to about 5:1, or from about 7:4 to about 5:1, or from about 5:4 to about 5:1, or from about 3:4 to about 5:1, or from about 1:4 to about 5:1, or from about 9:5 to about 5:1, or from about 8:5 to about 5:1, or from about 7:5 to about 5:1, or from about 6:5 to about 5:1, or from about 4:5 to about 5:1, or from about 3:5 to about 5:1, or from about 2:5 to about 5:1, or from about 1:5 to about 5:1, or from about 7:6 to about 5:1, or from about 5:6 to about 5:1, or from about 1:6 to about 5:1, or from about 9:7 to about 5:1, or from about 8:7 to about 5:1, or from about 6:7 to about 5:1, or from about 5:7 to about 5:1, or from about 4:7 to about 5:1, or from about 3:7 to about 5:1, or from about 2:7 to about 5:1, or from about 1:7 to about 5:1, or from about 9:8 to about 5:1, or from about 7:8 to about 5:1, or from about 5:8 to about 5:1, or from about 3:8 to about 5:1, or from about 1:8 to about 5:1, or from about 8:9 to about 5:1, or from about 7:9 to about 5:1, or from about 5:9 to about 5:1, or from about 4:9 to about 5:1, or from about 2:9 to about 5:1, or from about 1:9 to about 5:1, or from about 9:1 to about 5:2, or from about 8:1 to about 5:2, or from about 7:1 to about 5:2, or from about 6:1 to about 5:2, or from about 5:1 to about 5:2, or from about 4:1 to about 5:2, or from about 3:1 to about 5:2, or from about 2:1 to about 5:2, or from about 9:2 to about 5:2, or from about 7:2 to about 5:2, or from about 5:2 to about 5:2, or from about 3:2 to about 5:2, or from about 5:2 to about 5:2, or from about 9:3 to about 5:2, or from about 8:3 to about 5:2, or from about 7:3 to about 5:2, or from about 5:3 to about 5:2, or from about 4:3 to about 5:2, or from about 2:3 to about 5:2, or from about 1:3 to about 5:2, or from about 9:4 to about 5:2, or from about 7:4 to about 5:2, or from about 5:4 to about 5:2, or from about 3:4 to about 5:2, or from about 1:4 to about 5:2, or from about 9:5 to about 5:2, or from about 8:5 to about 5:2, or from about 7:5 to about 5:2, or from about 6:5 to about 5:2, or from about 4:5 to about 5:2, or from about 3:5 to about 5:2, or from about 2:5 to about 5:2, or from about 1:5 to about 5:2, or from about 7:6 to about 5:2, or from about 5:6 to about 5:2, or from about 1:6 to about 5:2, or from about 9:7 to about 5:2, or from about 8:7 to about 5:2, or from about 6:7 to about 5:2, or from about 5:7 to about 5:2, or from about 4:7 to about 5:2, or from about 3:7 to about 5:2, or from about 2:7 to about 5:2, or from about 1:7 to about 5:2, or from about 9:8 to about 5:2, or from about 7:8 to about 5:2, or from about 5:8 to about 5:2, or from about 3:8 to about 5:2, or from about 1:8 to about 5:2, or from about 8:9 to about 5:2, or from about 7:9 to about 5:2, or from about 5:9 to about 5:2, or from about 4:9 to about 5:2, or from about 2:9 to about 5:2, or from about 1:9 to about 5:2, or from about 9:1 to about 5:3, or from about 8:1 to about 5:3, or from about 7:1 to about 5:3, or from about 6:1 to about 5:3, or from about 5:1 to about 5:3, or from about 4:1 to about 5:3, or from about 3:1 to about 5:3, or from about 2:1 to about 5:3, or from about 9:2 to about 5:3, or from about 7:2 to about 5:3, or from about 5:2 to about 5:3, or from about 3:2 to about 5:3, or from about 5:3 to about 5:3, or from about 9:3 to about 5:3, or from about 8:3 to about 5:3, or from about 7:3 to about 5:3, or from about 5:3 to about 5:3, or from about 4:3 to about 5:3, or from about 2:3 to about 5:3, or from about 9:4 to about 5:3, or from about 7:4 to about 5:3, or from about 5:4 to about 5:3, or from about 3:4 to about 5:3, or from about 1:4 to about 5:3, or from about 9:5 to about 5:3, or from about 8:5 to about 5:3, or from about 7:5 to about 5:3, or from about 6:5 to about 5:3, or from about 4:5 to about 5:3, or from about 3:5 to about 5:3, or from about 2:5 to about 5:3, or from about 1:5 to about 5:3, or from about 7:6 to about 5:3, or from about 5:6 to about 5:3, or from about 1:6 to about 5:3, or from about 9:7 to about 5:3, or from about 8:7 to about 5:3, or from about 6:7 to about 5:3, or from about 5:7 to about 5:3, or from about 4:7 to about 5:3, or from about 3:7 to about 5:3, or from about 2:7 to about 5:3, or from about 1:7 to about 5:3, or from about 9:8 to about 5:3, or from about 7:8 to about 5:3, or from about 5:8 to about 5:3, or from about 3:8 to about 5:3, or from about 1:8 to about 5:3, or from about 8:9 to about 5:3, or from about 7:9 to about 5:3, or from about 5:9 to about 5:3, or from about 4:9 to about 5:3, or from about 2:9 to about 5:3, or from about 1:9 to about 5:3, or from about 9:1 to about 5:4, or from about 8:1 to about 5:4, or from about 7:1 to about 5:4, or from about 6:1 to about 5:4, or from about 5:1 to about 5:4, or from about 4:1 to about 5:4, or from about 3:1 to about 5:4, or from about 2:1 to about 5:4, or from about 9:2 to about 5:4, or from about 7:2 to about 5:4, or from about 5:2 to about 5:4, or from about 3:2 to about 5:4, or from about 1:2 to about 5:4, or from about 9:3 to about 5:4, or from about 8:3 to about 5:4, or from about 7:3 to about 5:4, or from about 5:3 to about 5:4, or from about 4:3 to about 5:4, or from about 2:3 to about 5:4, or from about 1:3 to about 5:4, or from about 9:4 to about 5:4, or from about 7:4 to about 5:4, or from about 5:4 to about 5:4, or from about 3:4 to about 5:4, or from about 5:4 to about 5:4, or from about 9:5 to about 5:4, or from about 8:5 to about 5:4, or from about 7:5 to about 5:4, or from about 6:5 to about 5:4, or from about 4:5 to about 5:4, or from about 3:5 to about 5:4, or from about 2:5 to about 5:4, or from about 1:5 to about 5:4, or from about 7:6 to about 5:4, or from about 5:6 to about 5:4, or from about 1:6 to about 5:4, or from about 9:7 to about 5:4, or from about 8:7 to about 5:4, or from about 6:7 to about 5:4, or from about 5:7 to about 5:4, or from about 4:7 to about 5:4, or from about 3:7 to about 5:4, or from about 2:7 to about 5:4, or from about 1:7 to about 5:4, or from about 9:8 to about 5:4, or from about 7:8 to about 5:4, or from about 5:8 to about 5:4, or from about 3:8 to about 5:4, or from about 1:8 to about 5:4, or from about 8:9 to about 5:4, or from about 7:9 to about 5:4, or from about 5:9 to about 5:4, or from about 4:9 to about 5:4, or from about 2:9 to about 5:4, or from about 1:9 to about 5:4, or from about 9:1, 8:1, 7:1, 6:1, 5:1, 4:1, 3:1, 2:1, 1:1, 9:2, 7:2, 5:2, 3:2, 8:3, 7:3, 5:3, 4:3, 2:3 or 1:3, 9:4 7:4, 5:4, 3:4, 1:4, 9:5, 8:5, 7:5, 6:5, 4:5, 3:5, 2:5, 1:5, 7:6, 5:6, 1:6, 9:7, 8:7, 6:7, 5:7, 4:7, 3:7, 2:7, 1:7, 9:8, 7:8, 5:8, 3:7, 1:8, 8:9, 7:9, 5:9, 4:9, 2:9, 1:9, 5:1, 5:2, 5:3, 5:4, 4:1, 4:3, 3:1, 3:2, 3:4, 3:5, 2:1, 2:3, or 2:5. Preferably, the weight ratio is 3:2 or 7:3 or 8:2, which represent cases where the artesunate salt is about 60% of the weight versus 40% for the emulsifying agent, or 70% of the weight versus 30% for the emulsifying agent, or 80% of the weight versus 20% for the emulsifying agent, respectively. A 3:2 weight ratio represents approximately a 1:1 molar ratio, a 7:3 weight ratio represents approximately a 1.7:1 molar ratio, and a 8:2 weight ratio represents approximately a 2.9:1 molar ratio when the artesunate is a sodium salt and the emulsifying agent is sodium lauryl sulfate (SLS).

According to another embodiment, the emulsifying agent is a surfactant. The surfactant may be a nonionic, an anionic, a cationic, an amphoteric surfactant, or a combination thereof. The surfactant may be selected from the group consisting of sodium lauryl sulfate, sorbitan stearate, sorbitan esters, sodium laureth sulfate, sarkosyl. cocamidopropyl betaine (CAPB), sodium lauryl ether sulfonate, alkyl benzene sulfonates, nonylphenol ethoxylate, hexadecylbetaine, lauryl betaine, ether ethoxylate, Sodium Myristyl Sulfate, polysorbate 20, polysorbate 80, lecithin, Octyl phenol ethoxylate (Triton X-100), glyceryl monostearate, 3-[(3-Cholamidopropyl)dimethylammonio]-1-propanesulfonate (CHAPS). The surfactant may be preferably sodium lauryl sulfate.

According to another embodiment, the emulsifying agent may be a cholate. The cholate may be selected from the group consisting of cholic acid, glycocholic acid, taurocholic acid, deoxycholic acid, chenodeoxycholic acid, glycochenodeoxycholic acid, taurochenodeoxycholic acid and lithocholic acid.

According to another embodiment, the pH value may be obtained with a weak base. For example, the weak base may be a carbonate salt, such as sodium carbonate (Na₂CO₃), potassium carbonate (K₂CO₃), sodium bicarbonate (NaHCO₃), and potassium and bicarbonate (KHCO₃).

According to another embodiment, the extended release gastro retentive dosage form may further comprise a proton pump inhibitor. The proton pump inhibitor is omeprazole, lansoprazole, dexlansoprazole, rabeprazole, pantoprazole, esomeprazole, omeprazole, or combinations thereof.

According to another embodiment, the extended release gastro retentive dosage form may further comprise an antibiotic. The antibiotic may be amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

According to another embodiment, the extended release gastro retentive dosage form of the presente invention may comprise the carboxylated polysaccharide and cinnamaldehyde conjugate described herein and further comprise the extended release gastro retentive dosage form comprising the artesunate emulsion, of the present invention.

### USE OF THE COMPOSITION OF THE PRESENT INVENTION

Treatment of H. pylori Infection Commonly Recommended by Health Care Practitioners

The composition of the present invention may be useful in the prevention and/or treatment of Helicobacter pylori infections, gastric ulcers, gastric cancer, and combinations thereof.

According to an embodiment, there is provided a method for prevention or treatment of Helicobacter pylori infection, gastric ulcers, or a combination thereof comprising administering to a subject in need thereof a therapeutically effective amount of the extended release gastro retentive dosage form of the present invention.

Also, according to another embodiment, there is provided the use of an extended release gastro retentive dosage form of the present invention for the prevention or treatment of Helicobacter pylori infection, gastric ulcers, or a combination thereof in a subject in need thereof.

Also, according to another embodiment, there is provided the use of an extended release gastro retentive dosage form of the present invention for the preparation of a medicament for the prevention or treatment of Helicobacter pylori infection, gastric ulcers, or a combination thereof in a subject in need thereof.

Also, according to another embodiment, there is provided an extended release gastro retentive dosage form of the present invention for use for the prevention or treatment of Helicobacter pylori infection, gastric ulcers, gastric cancer or a combination thereof.

First-line eradication regimens achieve high rates of both eradication and patient compliance. Two triple therapy 14-day regimens are currently accepted as first-line therapy. They combine a proton pump inhibitor (PPI) with either «metronidazole and clarithromycin», or «amoxicillin and clarithromycin». These regimens generally achieve eradication rates of >80%. Since non-compliance can drastically reduce eradication rates, twice daily administration schedules are recommended.

Second-line eradication regimens include quadruple therapy with bismuth, metronidazole, and tetracycline plus either a PPI or an H2 receptor antagonist (H2RA) Cimetidine). If a PPI is chosen, the regimen can be given for 7 days; however, if an H2RA is used, 14 days are recommended. Quadruple therapies are considered second-line because the regimens require a more complex administration schedule (e.g. QID) and may be less well tolerated. Quadruple therapies are therefore usually reserved for patients who have failed one or more courses of triple therapy.

Main Factors to Eradicate Helicobacter pylori

Due to the appearance of H. pylori resistant to antibiotics commonly used (amoxicillin, clarithromycin and metronidazole), the association with other antibiotics is proposed such as rifabutin and amoxicillin (TALICIA®). Although these novel associations remain effective for the moment, the resistance to these combination therapy appeared almost as soon as that treatment was introduced as the official first line therapy and that it spread steadily and aggressively after that.

Current treatments to eradicate H. pylori include antibiotics based triple therapy, which bring some risk of untoward effects, particularly the multiple resistance of H. pylori to amoxicillin and tetracycline. Some of these strains are resistant to clarithromycin, metronidazole, and levofloxacin (Caliskan et al. 2015. Rev. Soc. Bras. Med. Trop., 48, 278-284). For this reason, natural agents may achieve the therapeutic goal of eradication without undue risks may achieve the therapeutic goal of eradication without undue risks.

It is worth mentioning that proton pump inhibitors are also involved in the combination for treatment and used not only to increase the pH of gastric acid, but also because of their anti-urease activity.

According to one embodiment of the present invention, there is disclosed the combination of natural products, particularly of EO (cinnamaldehyde), and artesunate, to reduce the use of antibiotics and to avoid the appearance other resistant to antibiotics.

According to an embodiment of the present invention, there is provided a conjugate of cinnamaldehyde directly on a negatively charged polymers, particularly carboxylated polysaccharide polymers, via acetal, hemiacetal or cyclic hemiacetal bonding.

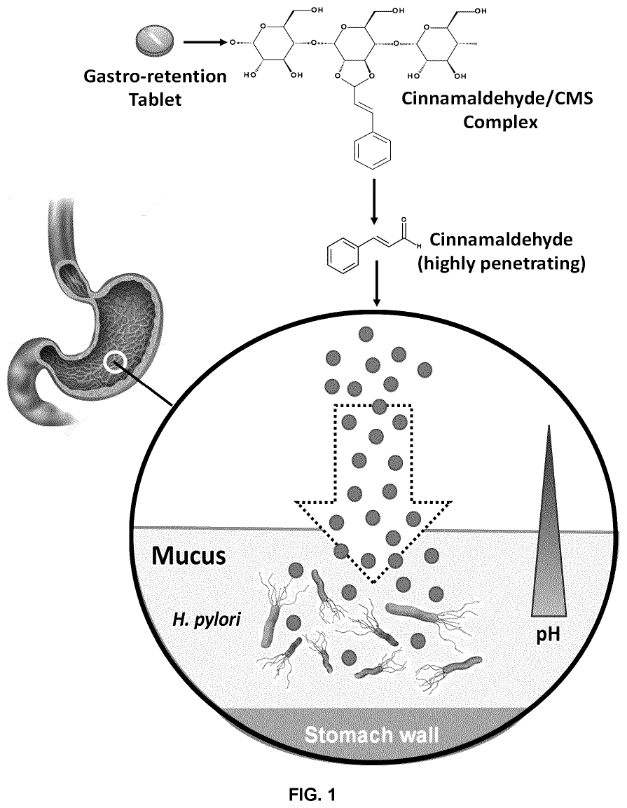

This conjugation permits not only to provide chemical stabilization, but also to prevent the rapid evaporation and degradation of cinnamaldehyde. Formulated under gastro-retention dosage form, the tablets remain stable in the stomach. After hydration, there is protonation on the hemiacetal and/or acetal bonding leading the release of the bioactive agent (FIG. 1). FIG. 2 illustrates the formation of such conjugate of cinnamaldehyde with carboxymethyl starch.

It is important to mention that the majority (approximately 80%) of infecting H. pylori are found living in gastric mucus rather than in contact with the underlying epithelium. In fact, the mucus layer that overlies the epithelial cells in the gastrointestinal tract is a physical barrier which acts to prevent pathogens from colonizing and interacting with the underlying epithelium. Pathogens which infect mucosal surfaces share two main goals: i) to overcome the mucus barrier; and ii) to interact with the underlying epithelial cells which results in disease. Globally, the mucus layer plays as a protective niche for H. pylori and this is an important component in the failure of antibiotic treatments.

The use a mucolytic agent in formulations were proposed, to generate a passage through mucosal layer promoting thus the entry of antibacterial agents to reach at the infection site of helicobacter. However, since the EO, particularly in the instant case cinnamaldehyde, is highly penetrating through the mucosal layer, no mucolytic agent is required (FIG. 1).

In another embodiment of the present invention, there is provided a water soluble artesunate, particularly provided a water soluble artesunate having increased solubility in gastric acid fluid. This particularity may permit the formulation of an extended release tablet or capsule which can remain in stomach by floating or swelling, for a long period to deliver the API at the infection site of Helicobacter pylori infection. Additionally, using artesunate, an endoperoxide molecule able to generate ROS, is believed to be the underlying mechanism involved in artesunate-mediated bacterial cell death.

Important Parameters to Successful Eradicate H. pylori Infection

Several parameters are established in the present application to successful treatment the H. Pylori infection:

### Extended Release Gastro-Retention Dosage Forms

Since H. pylori is colonizing the stomach, a gastro-retention tablet dosage form is proposed to achieve a successful treatment. These gastro-retention dosage forms could be done according to several method. The present invention preferably uses floating or swelling tablet formulation. It is also included a mucoadhesive drug delivery system, which uses bioadhesive polymers, such as carboxylated polymers.

### Water Soluble Bioactive Agents

Many bioactive agents are lipophile and generally insoluble or poorly dispersible and agglomerated in the gastric fluid. These phenomena also induce a slow disintegration of bioactive lipid agents from the solid tablet dosage form and constitutes the limiting step for its efficacy. Certain bioactive lipid agents, under oil liquid form such as essential oils, remain generally on the surface of the gastric fluid until gastric emptying to reach the intestine tract. In this case, there is no or very little contact of the bioactive lipid agents with bacteria, which thus reduces greatly their efficacy. For this reason, cinnamaldehyde and artesunate used in the present invention used in the present application are conjugate or coordinated with the carboxylated polymers to enhance their water-solubility, and consequently their efficacy.

### Urease Inhibitors and Proton Pump Inhibitors

According to further embodiments, the methods of the present invention, use of the present invention and extended release gastro retentive dosage form for use of the present invention may further comprising the use of a proton pump inhibitor.

Urease enzymes catalyze the hydrolysis of urea into carbon dioxide and ammonia. Urease is found in bacteria, and also in mammals and humans. The presence of urease is considered to be very harmful to mammals, including humans, due to the production of the toxic ammonia product. However, mammalian cells do not produce urease, in fact, the sources are the various bacteria in the body, specifically in the intestine.

Therefore, the addition of anti-urease (also known as urease inhibitor) compound in the composition of the present invention is helpful indispensable to successfully treat H. pylori. Examples of anti-urease include but are not limited to acetohydroxamic, hydroxyurea, and plant extracts from Eucalyptus, and Taraxacum.

Alternatively, in embodiments, the dosage forms of the present invention may also comprise a proton pump inhibitor (PPI), such as omeprazole or pantoprazole. Suitable proton pump inhibitor includes but are not limited to omeprazole, lansoprazole, dexlansoprazole, rabeprazole, pantoprazole, esomeprazole, omeprazole, or combinations thereof. Proton pump inhibitors may be present in the dosage form of the present invention in therapeutically effective quantities already known for these compounds. For example, omeprazole may be used at a dose of 20 or 40 mg per dosage form. Pantoprazole may be used at a dose of 20 or 40 mg per dosage form.

In the method or use of the present invention, the proton pump inhibitor may be administered before, or at the same time the extended release gastro retentive dosage form. According to embodiments, the proton pump inhibitor may be administered (or used) about 10 mins to about 60 mins, or about 20 mins to about 60 mins, or about 30 mins to about 60 mins, or about 40 mins to about 60 mins, or about 50 mins to about 60 mins, or about 10 mins to about 50 mins, or about 20 mins to about 50 mins, or about 30 mins to about 50 mins, or about 40 mins to about 50 mins, or about 10 mins to about 40 mins, or about 20 mins to about 40 mins, or about 40 mins to about 50 mins or about 10 mins to about 30 mins, or about 20 mins to about 30 mins, or about 10 mins to about 20 mins before the extended release gastro retentive dosage form, or about 30 minutes before the extended release gastro retentive dosage form.

### Antibiotics

According to further embodiments, the methods of the present invention, use of the present invention and extended release gastro retentive dosage form for use of the present invention may further comprising the use of an antibiotic.

In embodiments, the dosage forms of the present invention may also comprise an antibiotic. Suitable antibiotics include but are not limited to amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof. Antibiotics may be present in the dosage form of the present invention in therapeutically effective quantities already known for these compounds. For example, 1000 mg clarithromycin, or 500 mg amoxicillin may be used per dosage form.

The antibiotic may be administered or used before, after, or at the same time as the extended release gastro retentive dosage form. The antibiotic may be administered at the same time as the extended release gastro retentive dosage form. The proton pump inhibitor may be administered at the same time or used before the extended release gastro retentive dosage form, and the antibiotic may be administered at the same time or after the extended release gastro retentive dosage form.

Combination with other Bioactive Agents

According to another embodiment, the combination with other natural bioactive agents may be of interest to improve the treatment efficacy. Generally, the bioactive agent combination can direct at different targets (multitarget) of bacteria. For example, cinnamaldehyde can disrupt the bacteria membrane and inhibit urease, whereas artesunate complex generated ROS which deteriorate vital structure of pathogens or its virulence factors such as Cytotoxine-associated gene A (CagA), Vacuolating cytotoxin A (VacA) and cell surface adhesin (BabA), etc. Although many virulence factors are not indispensable, their inhibition can sufficiently attenuate infectivity to allow stand-alone therapy or can provide strong synergy with classic antibiotics, thereby increasing specificity and eradication efficiency of the therapy.

Additionally, the cinnamaldehyde can act as a penetrating enhancer agent favorized the penetration of the other bioactive agents at the H. pylori infection site. By example in the present invention, the combination of Cinnamaldehyde/Starch Glycolate complex with an antibiotic such as Amoxicillin permits to successful eradicate H. pylori in mice. As mention previously, Cinnamaldehyde is released from tablet and penetrated through the mucus, and at the same time generate a path for amoxicillin to reach at the H. pylori colonized site.

### Process for Preparation of Water Soluble Artesunate

According to another embodiment, there is disclosed a process for the preparation of an extended release gastro retentive formulation comprising an artesunate emulsion comprising the steps of:
a) introducing an artesunate or pharmaceutically acceptable salts thereof, or stereoisomers thereof in a buffered solution comprising an emulsifying agent, to obtain an emulsified artesunate solution;
b) adjusting pH value of the emulsified artesunate solution to a pH value of about 7.5 to about 7.9, to obtain an adjusted emulsified artesunate solution; and
c) drying the adjusted emulsified artesunate solution, to obtain a dry powder of emulsified artesunate, having a pH value of from about 7.5 to 7.9.

The pH value of the dry powder of emulsified artesunate may be from about 7.5 to about 7.9, or from about 7.6 to about 7.9, or from about 7.7 to about 7.9, or from about 7.8 to about 7.9, or from about 7.5 to about 7.8, or from about 7.6 to about 7.8, or from about 7.7 to about 7.8, or from about 7.5 to about 7.7, or from about 7.6 to about 7.7, or from about 7.5 to about 7.6, or about 7.5, 7.55, 7.6, 7.65, 7.7, 7.75, 7.8, 7.85, or 7.9.

The weight ratio of the artesunate pharmaceutically acceptable salt and the emulsifying agent is from about 9:1 to about 1:1 (see also the enumeration above). Preferably, the ratio is 7:3 or 3:2.

The emulsifying agent may be a surfactant. The surfactant may be a nonionic, an anionic, a cationic, an amphoteric surfactant, or a combination thereof. The surfactant may be selected from the group consisting of sodium lauryl sulfate, sorbitan stearate, sorbitan esters, sodium laureth sulfate, sarkosyl, cocamidopropyl betaine (CAPB), sodium lauryl ether sulfonate, alkyl benzene sulfonates, nonylphenol ethoxylate, hexadecylbetaine, lauryl betaine, and ether ethoxylate, Sodium Myristyl sulfate, polysorbate 20, polysorbate 80, lecithin, Octyl phenol ethoxylate (Triton X-100), glyceryl monostearate, 3-[(3-Cholamidopropyl)dimethylammonio]-1-propanesulfonate (CHAPS). The surfactant may be sodium lauryl sulfate.

The emulsifying agent may be a cholate. The cholate may be selected from the group consisting of cholic acid, glycocholic acid, taurocholic acid, deoxycholic acid, chenodeoxycholic acid, glycochenodeoxycholic acid, taurochenodeoxycholic acid and lithocholic acid.

The pH value may be obtained with a weak base, such as a carbonate salt. Carbonate salts may be selected from the group consisting of sodium carbonate (Na₂CO₃), potassium carbonate (K₂CO₃), sodium bicarbonate (NaHCO₃), potassium and bicarbonate (KHCO₃), and combinations thereof.

The drying may be by spray drying.

The present invention will be more readily understood by referring to the following examples which are given to illustrate the invention rather than to limit its scope.

### EXAMPLE 1

### Preparation Of Conjugation Complex

To prepare the water-soluble solid powder forms of the present invention, either in the form of the cinnamaldehyde conjugation complex, the following steps may be followed:
a) Providing from 0.1% to 10% of at least an oil liquid bioactive lipid agent selected from cinnamaldehyde;
b) Providing emulsifying agent possessing a melting point at least 100° C. or more in an appropriate volatile solvent (preferably cholic, deoxycholic or derivative bile acids and its salts, in methanol) or glyceryl monooleate;
c) Providing a mixture of hydrophilic polymer (starch glycolate or CMS, CM-cellulose, etc.) having a negative charge and at least one carboxyl functional groups, and having at least one or more hydroxyl group which can interact with cinnamaldehyde to form an acetal, hemiacetal or cyclic hemiacetal; and having at least a carboxyl/carboxylate.
d) Introduce a) in b) and mix until obtaining a homogenized mixture ab.
e) Spray the solution a+b directly on the mixture of c) and drying at room temperature or aerating to remove the solvent in excess.

### Preparation of Carboxymethyl-Starch

The sodium starch glycolate (SG) is synthesized by etherification of starch with by using sodium chloroacetate as functionalizing agent in alkaline condition. The reaction medium is a mixture of isopropanol/water (80:20 v/v) or ethanol/water (80:20, v/v). Soluble potato starch and sodium monochloroacetate were purchased from Sigma-Aldrich (St. Louis, Mo., USA). The other chemicals were of reagent grade and used without further purification.

Practically, an amount of 160 g of soluble potato starch (potato starch, Sigma, Saint Louis, Mo., USA or ClearGum PB-99-EXP, Roquette, Lestrem, France) is introduced into a two-liter beaker. Then, a volume of 1200 mL of a mixture of isopropanol/water (85:15, v/v) are added to the beaker, under stirring at room temperature. After 15 minutes, NaOH pellets are added in the solution to obtain a final concentration about of 2.0 (maximum 3.0 M) and the stirring is continued until NaOH pellets are completely dissolved. The reaction of carboxymethylation is started by addition an amount of 150 g of sodium monochloroacetate, always under stirring for one hour and, before left overnight at room temperature (22° C.), a volume of 400 mL of methanol is added to avoid the gelatinization or aggregation.

At the end of the reaction, the stirring is stopped to separate SG from the supernatant by decantation. SG is washed by adding an excess (about 2.0 L) of methanol/water (80:20, v/v) solution. The precipitated product, SG, was collected by filtration on a Whatman cellulose filter paper and the washing is repeatedly at least three times with methanol/water at (80:20, v/v), (90/10) and (95/5) ratio, respectively. The SG mass was finally dehydrated in 2 L of pure methanol (or ethanol) and air-dried to eliminate residual solvent to obtain the powder.

The degree of substitution (DS) was determined by the titrimetric method. In fact, the carboxyl groups of the carboxymethyl-starch are first converted into the acidic (protonated) form by treatment of 1.0-g powder in ethanol solution containing HCl (5 mL concentrated HCl with 95 mL absolute ethanol) for 30 min. The protonated starch glycolate is then filtered, washed several times with ethanol/distilled water (90:10) to completely remove the acid excess, and washed with pure acetone for drying. Finally, an amount of 100 mg of protonated starch glycolate is suspended in 100 mL distilled water and titrated with a 0.05 M sodium hydroxide solution. The DS of SG is estimated about of 0.56.

### Preparation of Cinnamaldehyde/Starch Glycolate Complex

An amount of 6.0 g of deoxycholic acid (sodium salt form) is introduced in 20 mL of methanol and homogenized under mild stirring, at room temperature. Thereafter, an amount of 4.0 of trans-cinnamaldehyde is dispersed in the solution, always under stirring at room temperature.

After 5.0 minutes mixing, the trans-cinnamaldehyde/deoxycholate emulsifying solution is directly vaporized on the surface of 30 g of SG. It is important to shake and mix the powder to distribute evenly and to favor the absorption of trans-cinnamaldehyde/deoxycholate solution into the SG granules. The corresponding powders containing about 10% (w/w) of cinnamaldehyde are dried, closed tightly and stored in the dark place at room temperature.

### Characterization of Cinnamaldehyde/SG Complex

FTIR Analysis of Cinnamaldehyde/SG Complex

FT-IR spectra were recorded with a Spectrum One™ (Perkin-Elmer Instruments, Norwalk, USA) equipped with a Universal Attenuated Total Reflectance (UATR) device for powder analysis on the spectral region 4000-650 cm⁻¹ with 24 scans at 4 cm⁻¹ resolution. All spectra were normalized over the range using the Spectrum™ software 3.02. The samples were directly analyzed under powder form which was obtained by direct compaction (2.3 tons/cm²) of the powder in flat-faced punches with 12 mm diameter using a hydraulic press (Carver, Wabash, Ind., USA).

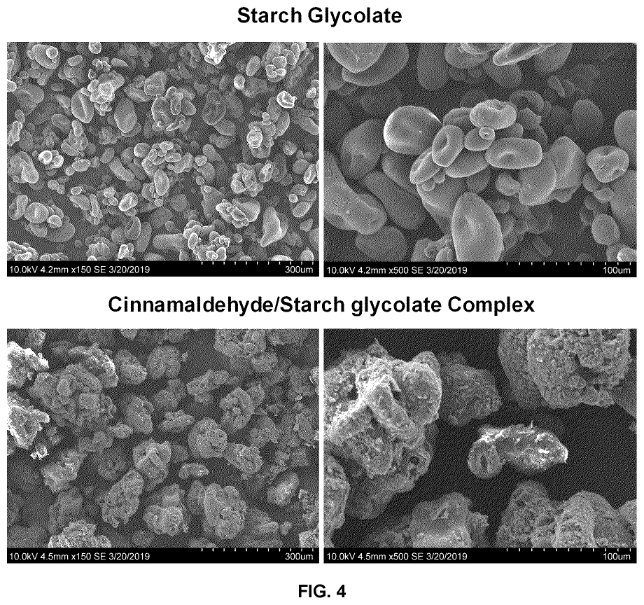

For FTIR spectra (FIG. 4) showed that following carboxymethylation, the SG presented absorption bands located at 1590 and 1410 cm⁻¹ asymmetric and symmetric stretching vibrations assigned to carboxylate anion groups.

When complexed cinnamaldehyde with SG, an increase in intensity of absorption bands located in spectral region of 2930-2860 cm⁻¹ assigned to C—H stretching vibrations is observed. Additionally, a new appearance of absorption band attributed to C═C from cinnamaldehyde is detected at 1675 cm⁻¹.

### SEM Analysis Cinnamaldehyde/SG Complex

The morphology and surface characteristics of samples are examined at various magnifications (150-500) with a S-3400N Variable Pressure SEM (JEOL Ltd., Tokyo, JP). The images are obtained with voltages of 10 kV and high vacuum.

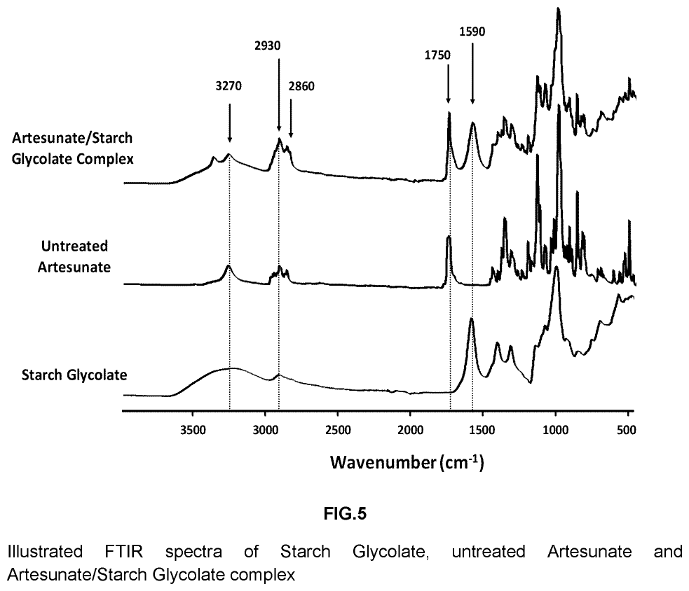

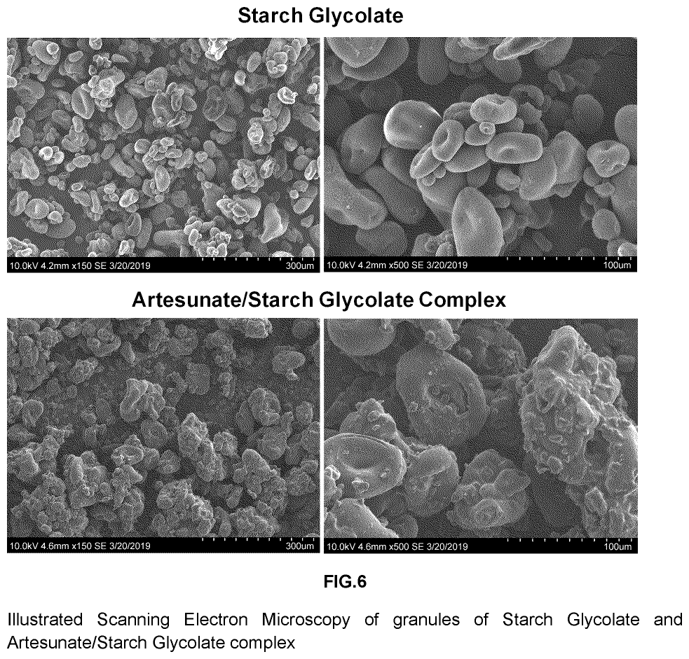

The morphology of SG granules in SEM micrographs are characterized by different shape and moderately smooth surface, ovoid or pear-shape, larger and greater granules between 10-80 μm in size (FIG. 5). The granules have eccentric holes and clearly visible. When complexed with cinnamaldehyde, SG granules are slightly larger than untreated and remained adhered to each other forming grapes. Generally, the complex possesses an irregular shape and rough surface.

### EXAMPLE 2

### Preparation of Artesunate

### Preparation of Artesunate Under Sodium Carbonate Salt Form

An amount of 18 g of artesunate is dispersed in 54 mL of water and 33 mL of aqueous sodium bicarbonate solution (4.16%) is then added. The solution is stirred mildly for 20 minutes at 25° C., filtered and then freeze-dried to provide a white crystalline powder.

### Immobilisation of Artesunate in Starch Glycolate by Entrapment

An amount of 10 g of water soluble artesunate is dispersed in 50 mL of ethanol and homogenized until a clear solution is obtained. The solution is sprayed directly on the surface of the starch glycolate powder (40 g) using an atomizer, similarly to a fluid bed granulation process.

### Preparation of Water-Soluble Artesunate Solid Powder

Different weight ratio of artesunate and emulsifying agent can be prepared, for example from 9:1 to 1:1). Preferably the artesunate/emulsifying agent weight ratio is about 3:2 (60% vs 40% of the weight, respectively). To prepare the water-soluble artesunate solid powder forms of the present invention, the following steps may be performed:

### Preparation of Artesunate Emulsion Solution

1. Disperse an amount of 24 g of sodium lauryl sulfate (SLS, 0.17 M) in 400 mL distilled water heated at 40-50° C. under mild stirring;
2. Add 11.2 g of sodium carbonate (0.21 M) to the solution and let it cool to room temperature under stirring; Sodium bicarbonate (molar mass 84.01 g/mol) could be used and in this case, an amount of 55 mg of sodium bicarbonate (1.31 M) is required;
3. Introduce slowly 56 g of artesunic acid (0.29 M) in the solution and homogenize until the solution is clear;
4. Adjust pH value if necessary, to about 7.6-7.8, preferably pH 7.75;
5. Complete the solution with distilled water to obtain a final volume at 500 mL before drying.

A 3:2 weight ratio represents approximately a 1:1 molar ratio, a 7:3 weight ratio represents approximately a 1.7:1 molar ratio, and a 8:2 weight ratio represents approximately a 2.9:1 molar ratio when the artesunate is a sodium salt and the emulsifying agent is sodium lauryl sulfate (SLS).

Unexpectedly, the pH of the solution is very important. The acceptable pH values should be between 7.5-7.9, and preferably 7.6-7.8. Artesunate powder prepared at pH 8.0 or higher is sticking and has poor flowability, particularly when the drying is performed in a spray-dryer. In this case, it may be difficult to fill completely the artesunate powder quantity in the die to obtain tablets by compaction. The pH value of 7.4 or lower provide artesunate powders presenting good physical properties, but poorly soluble.

Weak bases are preferable to increase the pH of the artesunate solution to avoid the degradation of artesunate, because artesunic acid is very soluble in alkaline solutions, but hydrolyses also rapidly to DHA. Generally, carbonate salts such as sodium (or potassium) carbonate (Na₂CO₃) or sodium (or potassium) bicarbonate (NaHCO₃) are preferred. Stronger bases such as sodium hydroxide, potassium hydroxide or calcium hydroxide can degrade or hydrolyze artesunate during the preparation.

The sodium lauryl sulfate (SLS) is used as emulsifying agent to improve the solubility and the stability of artesunate. Furthermore, the emulsion permits to confine artesunate, separated from the environment by a protective emulsifying layer. Such a protective coating can extend shelf life, prevent exposure to gastric acid in stomach and delay the degradation of artesunate. Suitable complexes were prepared with several surfactants such as of sodium lauryl sulfate, sodium laureth sulfate, sodium myristyl sulfate, polysorbate 20, polysorbate 80, lecithin, Octyl phenol ethoxylate (Triton X-100), glyceryl monostearate, 3-[(3-Cholamidopropyl)dimethylammonio]-1-propanesulfonate (CHAPS). Mixtures of biliary salts (e.g. NACRES NA.24™) and sodium cholate were also successfully used to prepare the complex.

To obtain a dry powder artesunate from a fluid, several drying processing methods may be used, such as precipitation by using solvents (alcohol or acetone), the freeze-drying or lyophilization. But these processing methods are very long and difficult for industrial manufacturing. In the present application, the spray drying method is preferably used because it is a scalable process and it is widely used to produce dry pharmaceutical powders. Furthermore, spray-drying is rapid, fully automated, continuous, reproducible, single-step, and thus, scalable without major modifications.

### Spray-Drying to Obtain Water Soluble Artesunate Powders

The drying to obtain the water soluble artesunate solid powders was performed using a Pilotech™ YC-510 small spray dryer. The spray dryer was equipped with a 0.7 mm standard nozzle jet and operated using the following parameters: inlet temperature 150° C., outlet temperature 80-90° C., spray flow approximately 500 mL/h, and airflow setting at about of 40 m³/h. The suspension was mixed continuously during the drying process using a magnetic stirrer to ensure homogenous solution.

As per the above process, water soluble artesunate is stabilized under emulsified form and more resistant to gastric acid for a long period (>12 h) than artesunic acid or other known formulations. Formulated under extended release or floating tablet, this stable and soluble form may be useful to exert its antibacterial activity during gastric transit, because H. pylori mainly colonizes the gastric mucosa. For intracellular H. pylori, an absorption in the bloodstream is necessary. In this case, it is believing that this water soluble stable artesunate form needs a time longer than DHA for biotransformation increasing thus its half-life.

### Characterization

### Solubility

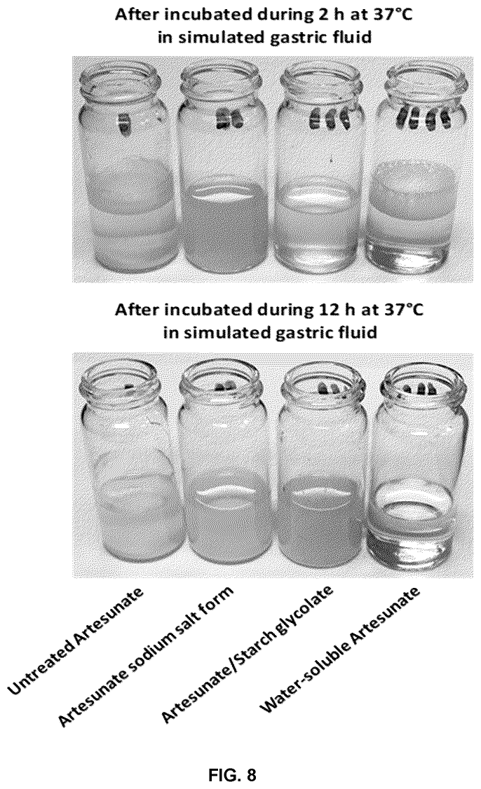

An amount of 0.5 g of different artesunate formulations (untreated artesunate, sodium artesunate, artesunate/SG complex and WSA powders) are dispersed in 10 mL of simulated gastric fluid (SGF, pH 1.2). All solutions are incubated at 36.5° C. under mild shaking with (100 rev/min) in a G24 Environmental Incubator Shaker (New Brunswick Scientific Co., N.J., USA). After 2 hours, as shown in FIG. 8 the solution of untreated artesunate has a milky appearance, with an important precipitation at the bottom of vial. For artesunate under sodium carbonate salt form, the solution is cloudy, but no evident precipitation is observed. In contrast, the solution of artesunate/SG complex and WSA are clear and transparent, and no precipitations are observed, indicating complete solubility. After incubation for 12 h at 36.5° C. in SGF, no change for untreated artesunate is detected. For the artesunate sodium salt form, a precipitation is formed at the bottom whereas the solution of artesunate/SG complex became cloudy. Only the WSA is unchanged and the solution remained clear. Therefore, in decreasing order, the most soluble is water-soluble artesunate, followed by the artesunate/SG complex, artesunate sodium salt form and untreated artesunate.

### FTIR Analysis

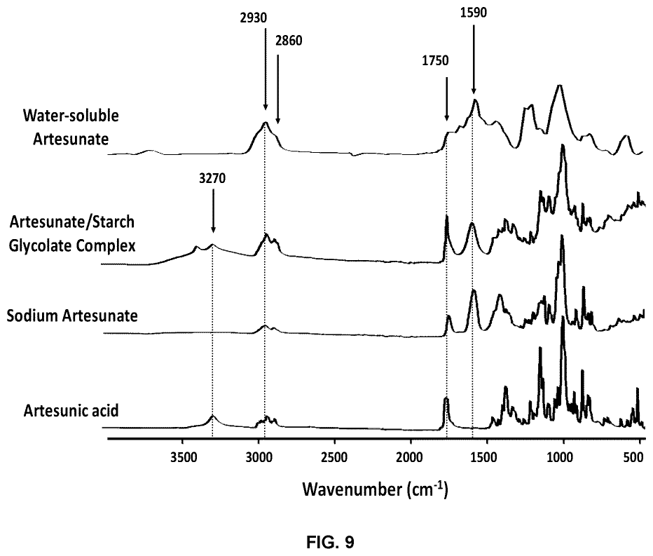

Now referring to the FIG. 9, typically absorption bands for artesunate are located 1750 cm⁻¹, assigned to carboxylic group from succinate residues. No absorption band at 1590 and 1410 cm⁻¹ is observed. This is due to the protonation of all carboxylate (—COO⁻) groups in carboxylic (—COOH) groups which is shifted from 1590 cm⁻¹ to 1750 cm⁻¹. For artesunate/SG complex, absorption bands located at 1750 and 1590 cm⁻¹ are respectively attributed for carboxylic group from artesunate and 1590 cm⁻¹ from starch glycolate.

As mention previously for WSA, this form is under pH value slightly alkaline (7.75) and stabilized by emulsion using sodium lauryl sulfate (SLS). For this reason, there is an increased intensity of the absorption bands located in spectral region of 2930-2860 cm⁻¹ assigned to alkyl (C-H) stretching vibrations. This increasing is mainly due to the presence alkyl chain of SLS. Additionally, an absorption band at 1750 cm⁻¹ attributed to carboxylic acid (—COOH) group and 1590 and 1410 cm⁻¹ to carboxylate (—COO⁻) group are observed. These results indicate clearly that WSA possesses two carboxyl forms. The main form is under succinate (carboxylate, —COO⁻) and the other is moderately under succinic acid (carboxylic, —COOH). The majority of succinate form explains why the WSA possesses a higher solubility than other artesunate treatments

### SEM Analysis

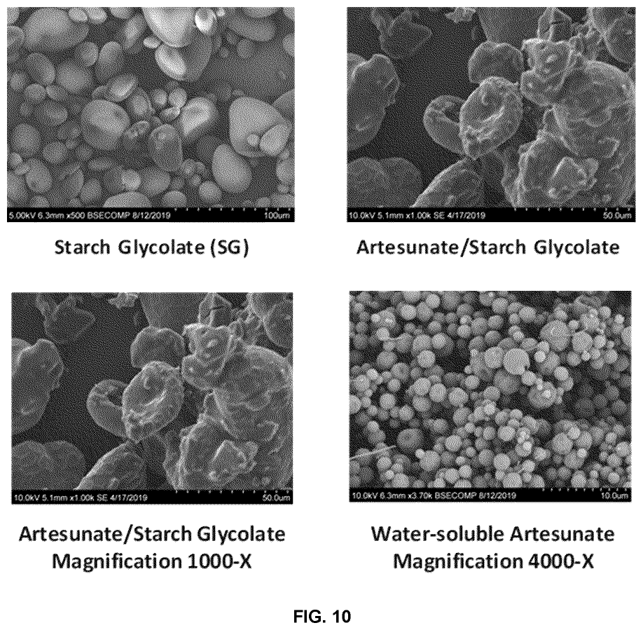

Now referring to FIG. 10, the morphology of artesunate/SG complex is similar to that of SG granules. However, artesunate/SG complex granules seemed larger than SG granules (mainly 20-40 μm versus 80-120 μm) and covered by small fine particles, thus generating a rough surface. For WSA, granules are spherical, and are smaller by about 40× than artesunate/SG complex granules.

### EXAMPLE 3

### AGAR Disk-Diffusion Test

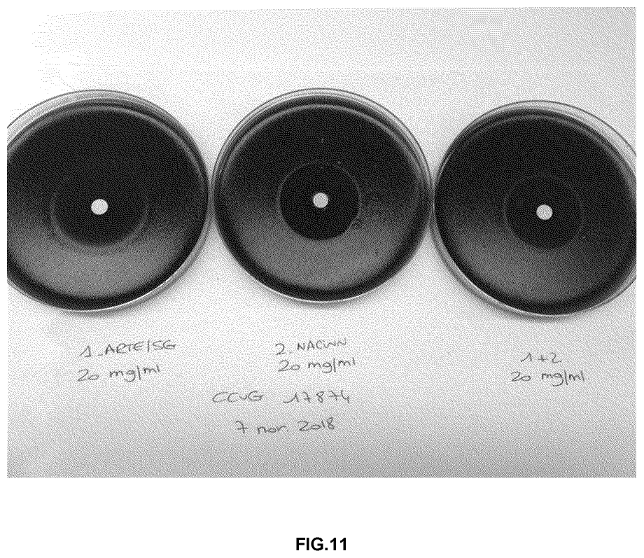

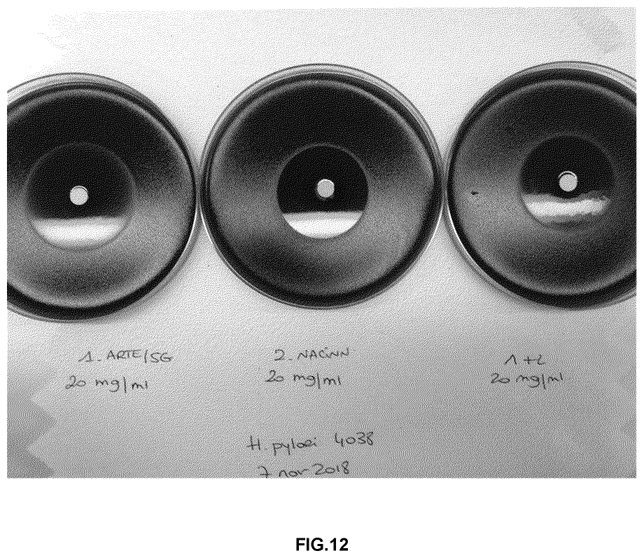

For Cinnamaldehyde/SG complex (1), artesunate untreated (2), artesunate/SG complex (3), WSA (4) and mixture (5) of Cinnamaldehyde/SG and of artesunate complex, these samples are dissolved in water at concentrations indicated in the FIGS. 11 and 12. For native (untreated) artesunate (0), the solvent used is methanol.

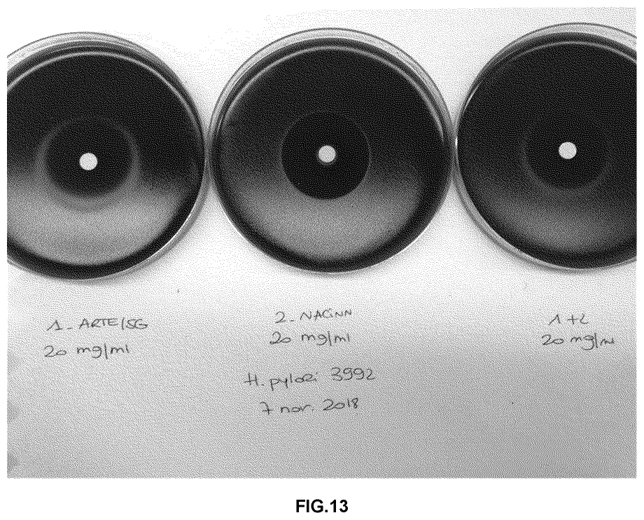

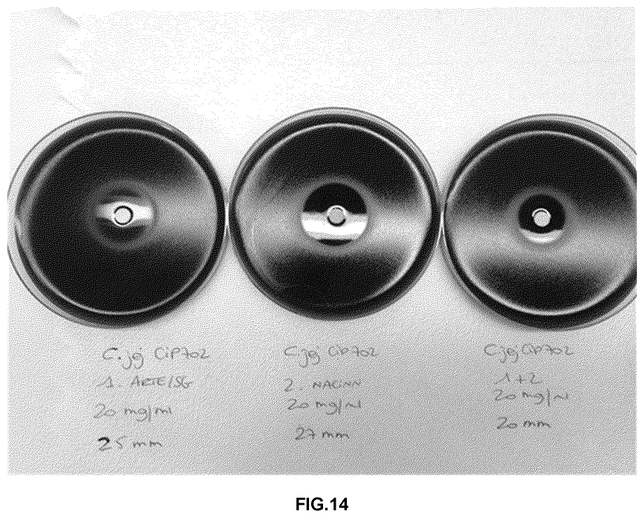

Practically, impregnated paper disks with a determined concentration (˜20-25 mg/mL) of antimicrobial agents are deposited on the surface of a standardized culture medium previously inoculated with a calibrated inoculum of a pure culture of the bacteria, Helicobacter pylori or Campylobacter. After incubation, the Petri dishes are examined and the diameters of the zones of inhibition surrounding the disks are measured and compared to the critical values of the different antimicrobial agents tested, to determine the clinical categorization (resistant, intermediate, sensitive). The diameter of the inhibition zone is proportional to the sensitivity of the bacterium tested. Results are represented in FIGS. 11-15. Generally, the results obtained show that the most effective antibacterial against Helicobacter pylori and Campylobacter pylori is Cinnamaldehyde/SG complex which possesses inhibition zones larger than other bioactive agents.

### EXAMPLE 4

### Minimal Inhibition Concentration (MIC) Determination

In total, about of 59 strains of Helicobacter pylori are selected for this study. Tested strains were strains isolated from gastric biopsies from different geographic origin: Africa (5 from Algeria and Congo), Asia (5 from Japan), America (3 from Costa Rica) and 45 strains isolated in France. The reference strain CCUG 17874 is also be included. These strains are maintained as stocks frozen at −80° C.

Identity of H. pylori strains to test

3770, 3392, 3995, 3997, 4001, 4010, 4011, 4013, 4015, 4023, 4025, 4028, 4029, 4030, 4032, 4035, 4037, 4038, 4039, 4040, 4063, 4064, LB016, LB024, ALG126, ALG140, AFR65, AFR69, AFR73, AFR76, AFR93, CR990922NN, CR12455, CR9909276, JAP09236, JAP09244, JAP09260, JAP TH79, JAP TH64, CCUG17874, 4820, 5019, 4805, 4784, 4898, 4977, 4682,4710, 4731, 4800, 4811, 4859, 4879, 4882, 4923, 4947, 4969, 4983, 5005, 5009 and 5021.

All these strains are provided by Centre National de Reference (C.N.R) des Campylobacters et Helicobacters and Centre Hospitalier Universitaire (CHU) de Bordeaux (Amélie Raba-Léon place, Bordeaux Cedex).

### Studied Bioactive Agents

1. Cinnamaldehyde/SG
2. Native artesunate
3. water soluble artesunate

### Determination of Minimum Inhibitory Concentrations

The antibacterial activity of these bioactive agents is determined by measuring the minimum inhibitory concentrations (MICs) of various H. pylori strains. MIC is performed by using the agar dilution method with the following parameters:

### Culture Medium

Mueller Hinton-2 agar (BioMérieux);

Sheep blood is added at 10% concentration, including globular extracts.

### Bacterial Inoculum

A suspension of approximately 10⁹ CFU/ml is prepared in brucella broth from a 48 h culture grown at 37° C. in microaerobic conditions.

### Preparation of Samples

A stock solution of 2 mg/mL of each compound is prepared in water (or in methanol for native artesunate). Then, the dilutions are prepared to a final concentration of 0.015 mg/L. In total, each plate contains 35.8 mL of agar, 4 mL of sheep blood with globular extract and 200 μL of sample solution which is poured into a 12 cm large Petri dish.

### Inoculation

The inoculation is performed with a multiple inoculator derived from a Steers' apparatus and the plates are incubated for 48 h at 37° C. in a workstation containing a microaerobic atmosphere (5% O₂, 10% CO₂, 85% N₂).

The MIC is determined as the lowest concentration of the bioactive agent which inhibit the growth of the H. pylori strain.

### Results

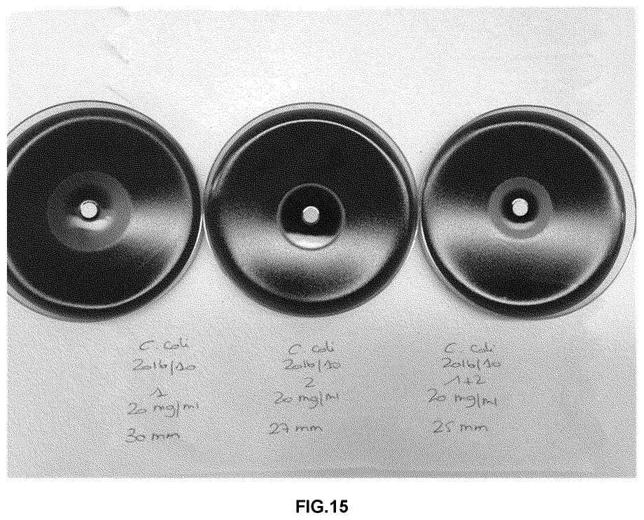

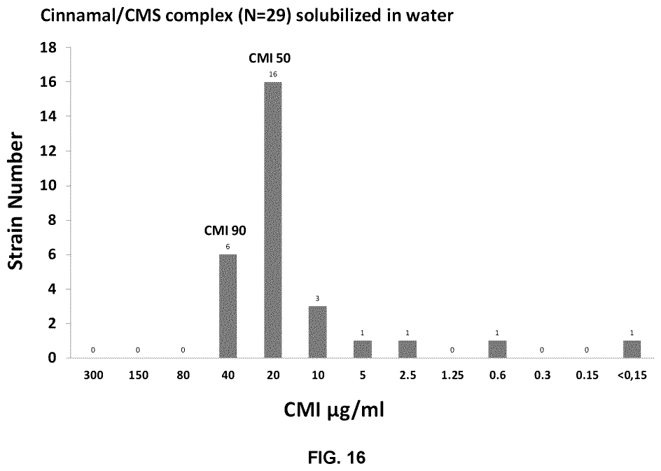

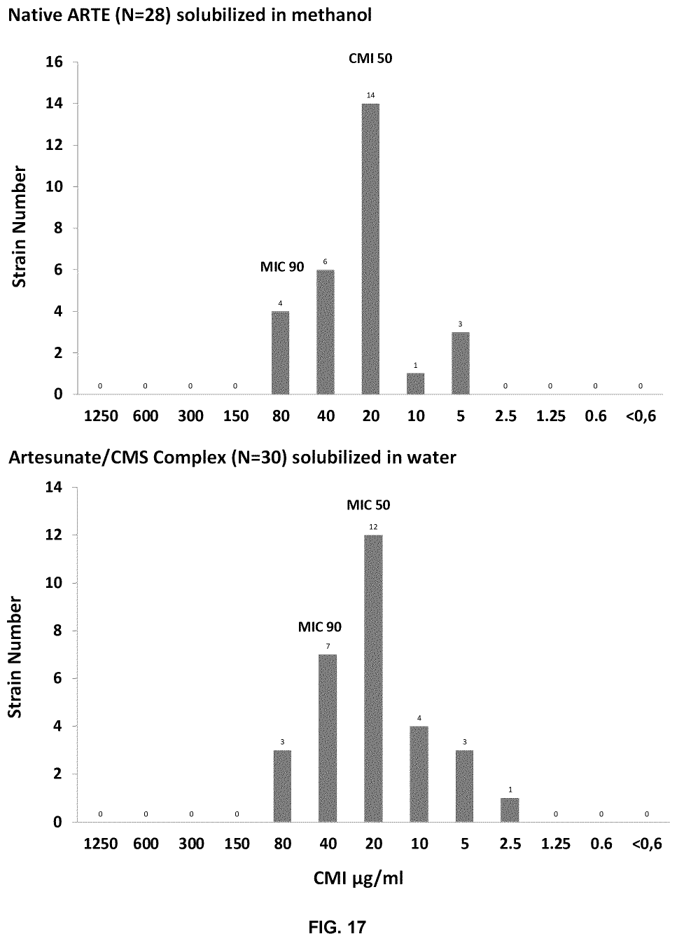

The MIC₅₀ and MIC₉₀ is represented for each compound or in combination in FIGS. 16 and 17.

Results show that the MIC 50 and 90 for Cinnamaldehyde/Starch Glycolate complex is respectively of 20 and 40 μg/mL. Similar results for artesunate/SG complex is noticed. However, the MIC 50 and 90 for native artesunate is respectively of 20 and 80 μg/mL.

### EXAMPLE 5

### Antibacterial Activity of Artesunate with Different Treatments

In this comparative study, the antibacterial activity of untreated Artesunate and Artesunate with different treatments (Sodium Artesunate, Artesunate/Starch glycolate and water-soluble Artesunate) are assessed with E. coli.

Practically, a bacterial cryovial is thawed and cultured twice during 18 h at 36.5° C. in 9 mL of broth for activation. Thereafter, a volume of 1 ml of culture (bacterial concentration of approximately 5.0×10⁷ CFU/mL) is gently mixed in 49 ml of medium containing native artesunate (0.2% w/w) to obtain a final bacterial concentration about of 1.0×10⁶ CFU/mL. Similar preparations for artesunate sodium salt form, artesunate/SG complex and WSA are carried out. After incubation during 18 h at 36.5° C., a volume of 1 mL of each tube is plated in Petri dish and incubated at 36.5° C. during 48 h.

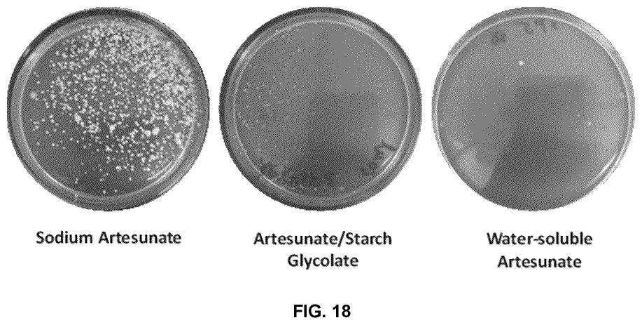

Now referring to FIG. 18, results indicate clearly that the antibacterial activity is essentially dependent on the solubility of artesunate. For untreated artesunate, no significant change is detected (not shown). However, for artesunate sodium salt form, a reduction from 10⁶ to 10⁴ CFU/mL is observed whereas artesunate/SG shows a reduction from 10⁶ to 10² CFU/mL. Only WSA provide a better result with a reduction from 10⁶ to <10 CFU/mL.

In this experiment, all conditions are similar, except the solubility of artesunate, which are different according to the method of preparation. Since WSA is very soluble in water, its antibacterial activity is higher and similarly for artesunate/SG. However, artesunate sodium salt form is only slightly soluble and for this reason it possesses a lower effectiveness.

Beside the factors mentioned previously, other factors are also important to eradicate Helicobacter pylori:
1. artesunate should be soluble in gastric acid;
2. artesunate should remain in the stomach for a long period, for example through floating or extended released formulations;
3. For intracellular H. pylori, it is depending the bioavailability of Artesunate, particularly its metabolites under active form (DHA) in the blood stream.

In the present application, WSA is prepared under sodium salt form to improve the solubility. Moreover, WSA is physically stable, particularly in gastric acid is due to the sodium lauryl sulfate (SLS) which confines artesunate from the environment by a protective emulsifying layer. Such a protective layer can extend shelf life, prevent the exposure to gastric acid in stomach and delays the degradation of artesunate. In contrast, artesunate currently in the market is sparingly solubility in water (0.68 mg/mL) and particularly less stable. Because of this poor stability, commercial artesunate is rapidly hydrolyzed to dihydroartemisinin (DHA) in the stomach when orally administrated. It is important to mention that DHA is also insoluble in gastric acid and its antibacterial activity is less important, according to the results detailed above. Also, DHA is the active metabolite. As soon as absorbed in the blood stream, DHA is rapidly converted to inactive metabolites (α-dihydroartemisinin-β-glucoronide) via glucuronidation catalyzed by UDP-glucuronosyltransferases, particularly UGT1A9 and UGT2B7. In contrast, artesunate/SG complex or WSA is soluble in gastric acid, are not only active to exert locally their antibacterial activity in the stomach, but they also remaining stable. Once passed through the liver, artesunate undergoes hepatic metabolism (biotransformation) to DHA before entering in the bloodstream. This step causes a delay which probably prolongs the half-life of DHA in the bloodstream.

### EXAMPLE 6

### Study of Pharmacokinetic of Artesunate

In this study, only artesunate/SG complex is selected in order to compare with untreated artesunate. The in vivo study was carried out on CD-1 mice (body weight approximately 20 g), and the experimental protocol is conducted according to the Animal Care Committee approval.

After review of the medical records provided by the animal supplier facility, mice are observed during 1 week for their acclimation before the experiment. Four groups (n=20 mice/group) are subjected to treatment as follows:
Group-1: untreated Artesunate administrated by IV at dose 3.5 mg/kg;
Group-2: Artesunate/Starch Glycolate administrated by IV at dose 3.5 mg/kg;
Group-3: untreated Artesunate orally administrated at dose 200 mg/kg; and
Group-4: Artesunate/Starch Glycolate orally administrated at dose 200 mg/kg.

After administering the treatments as indicated above, blood samples are collected at 5, 15, 30, 45, 120 and 480 minutes after administration and mice are sacrificed. The plasma is separated by centrifugation and conserved −80° C. before processing.

### Extraction of Samples

An amount of 400 μL of plasma is dispersed in 1 mL of acetonitrile to precipitate proteins and agitated during 5 minutes with a vortex before placing the suspensions of the sample in an ultrasonic bath for 1 minute. The precipitated proteins are separated by centrifugation (15000 g, 5 minutes at 16° C.) and the supernatant is collected in a microplate for analysis.

LC-MS/MS

The artesunate and its metabolites are determined using a liquid chromatography (UHPLC coupled with a triple quadrupole Shimadzu™ LC-MS 8030). The activity of the Artesunate, as well as their toxicity, is a result of their peroxide bridge. The metabolites therefore can be divided in biologically active hydroxylated compounds with an intact endoperoxide bridge and biologically inactive deoxy metabolites where the peroxide bridge has been reduced to an epoxide. Further all these metabolites undergo glucuronidation and are excreted in the urine or feces. Dihydroartemisinin appears to be the principal and/or primary metabolite of artesunate. The conversion (biotransformation) occurs to a varying degree and through different mechanisms. Dihydroartemisinin is thereafter converted to inactive metabolites via glucuronidation catalyzed by UDP-glucuronosyl transferases, UGT1A9 and UGT2B7. There are UGT1A1 and UGT1A8 that have also been reported to be involved. Dihydroartemisinin is also eliminated in bile as minor alucuronides, such as tetrahydrofuranoacetate.

To elucidate systematically the impact of artesunate metabolism and pharmacokinetics on drug response and adverse effects, the plasma concentrations of the parent drug and its two metabolites are determined.

### Optimization of the Parameters

A solution of the compound is prepared at 1 mg/mL in acetonitrile, and diluted 1/100 in an acetonitrile/water, 1:1 (v/v) ratio. The system is used in Flow Injection Analysis (FIA) mode, and a volume of 1 μL of the diluted solution is injected. All the conditions for analyzing are optimized such as i) the molecular ions (positive ion mode); ii) the m/z ratio of the molecular ions, the voltages (Q1, collision cell, Q3); iii) the fragment selection and the precursor ions (Multiple Reaction Monitoring) transition, etc. All water used in this experiment is deionized and purified.

### Optimization of the LC Method

A 10 mM solution in DMSO is diluted to a concentration of the order of micromolar (μM) to bring to the point of the analysis method. The chromatographic conditions are optimized (solvents, pH, elution mode, flow rate, etc). The chromatograms are recorded by preferably injecting 1 or 2 μL of solution.

TABLE 1
Optimization parameters for LC-MS/MS analysis
FLOW INJECTION METHOD

| | |
|---|---|
| Source | Electrospray Ionization (ESI) |
| Ionization | Positive |
| Molecular ion | m/z = 402.20 [M + H + NH₃]⁺ |
| Fragments | m/z = 267.20 163.20 |

LC MRM

| Column | Kinetex 2.6 μm C18 100A 50 × 2.1 mm (Phenomenex) | | | |
|---|---|---|---|---|
| Solvent A | Water 0.05% HCOOH | | | |
| Solvent B | Acetonitrile | | | |
| Debit | 0.5 mL/min | | | |
| Elution | Gradient | 0 min | 95% A | 5% B |
| | | 1.20-1.40 min | 5% A | 95% B |
| | | 1.42-2.80 min | 95% A | 5% B |

| | |
|---|---|
| Temperature | 40° C. |
| Retention | 1.35 min |
| time (tR) | |
| Extraction | 1 μL of solution 1 μM |

TABLE 2
Analytical Conditions

| Ionization | Transition MRM | | Column | Retention time (tR) |
|---|---|---|---|---|
| ESI | Precursor | Fragments | C18 | 1.35 min |
| Positive | 402.20 | 267.20 | 50 × 2.1 | |
| | | 163.20 | | |

Similar treatment for Dihydroartemisinin (DHA), and the fragmentation transitions for the multiple reaction monitoring were m/z 267.4-163.4. It is worthy to note that values of these fragments are close and difficult to differentiate. Generally, Artesunate is converted to DHA in liver and normally no trace of Artesunate is presented in the blood stream. In basing on the results obtained from external standard, the retention times (tR) are 1.35, 1.29 and 1.13 min respectively for Artesunate, DHA and DHA-Glucuronide.

### Preparation of Calibration Standards

Stock solutions of DHA is prepared by dissolving the accurately weighed reference compound in acetonitrile. The primary stock solution of DHA (400 μg/mL) is prepared in acetonitrile and diluted with acetonitrile to give working solutions of 0.05, 0.1, 0.5, 1.0, and 5 ng/mL. All stock solutions and working solutions are stored at 4° C.

### Results

Artesunate is rapidly hydrolyzed after intravenous administration to the active metabolite Dihydroartemisinin (DHA). For oral administration, artesunate is converted to DHA in the liver before entering the bloodstream. After, DHA is converted mainly to the biologically inactive metabolites, DHA-glucuronide via glucuronidation catalyzed by UDP-glucuronosyl transferases and excreted predominantly in urine.

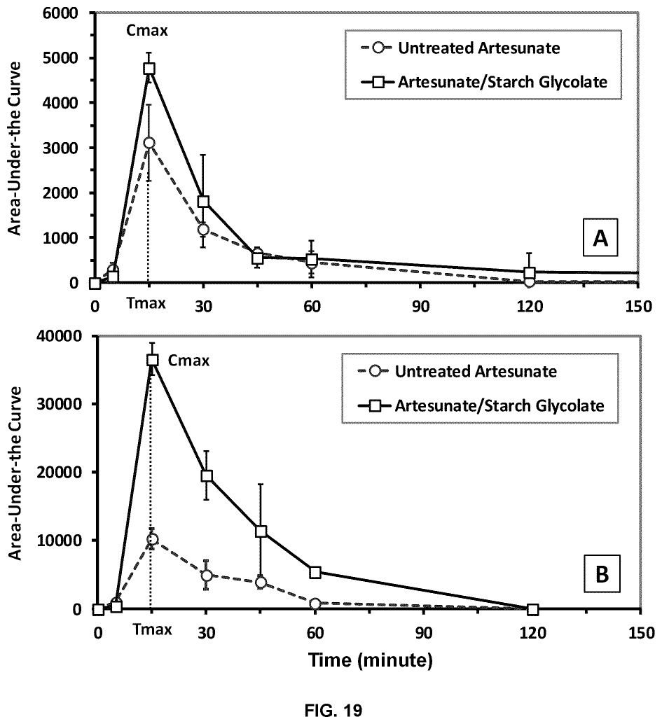

Plasma profile analysis is an important aspect in understanding biological handling of a given drug. All profiles after intravenous administration of artesunate show at least one distribution phase suggesting that more than one organ compartment exists. Now referring to FIG. 19, after intravenous administration of formulations, a rapid conversion of artesunate in active metabolite DHA by hydrolysis is observed and the maximal concentration (Cₘₐₓ) is reached after 15 min. Thereafter, a reducing in plasma concentration is indicative of rapid distribution of DHA into highly perfused organs such as brain, liver, prostate, etc. or possibly conversion of active DHA in inactive DHA-glucuronide.

For intravenous administration (Table 3), results show that there is significant difference of the DHA active metabolite. After 15 minutes, the maximal concentration (Cmax) of DHA is reached. For untreated artesunate, the Cₘₐₓ is about 0.06 nmol of DHA/mL whereas artesunate entrapped in SG is 0.09 nmol/mL.

The same profile is observed for inactive metabolite DHA-glucuronide which is most important for artesunate/SG. This observation is consistent, because artesunate/SG is more soluble, and its bioavailability is higher and thus inactivated more rapidly.

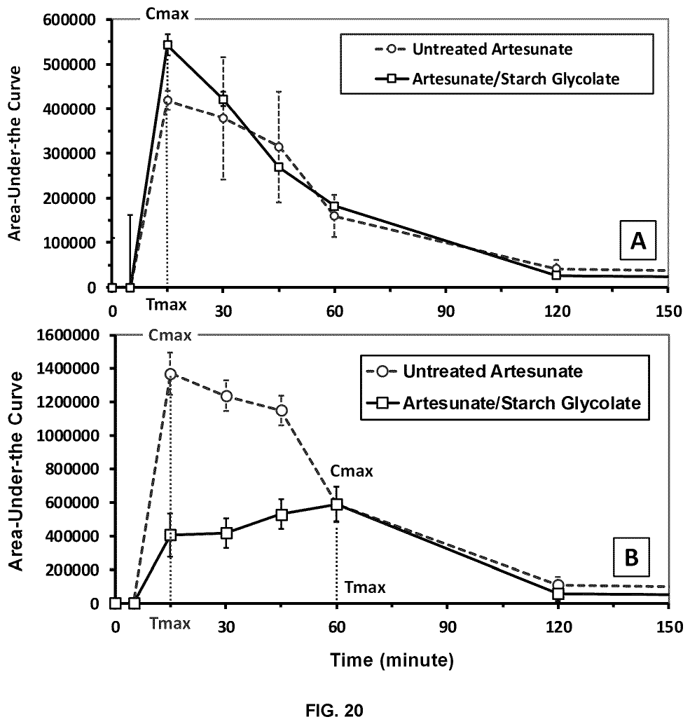

Now referring to FIG. 20, for oral administration, similar profile is noticed. A higher concentration of DHA is detected for artesunate/SG. However, a slowdown of the conversion of DHA in DHA-Glucuronate is observed for artesunate/SG. As mentioned previously, artesunate is susceptible to hydrolysis to DHA by gastric acid during transit the stomach. This active metabolite is directly absorbed in the bloodstream and rapidly converted to inactive metabolite DHA-Glucuronide. In contrast, artesunate/SG complex is stable in gastric acid and during the passage into the liver; there is a delay caused by biotransformation to DHA before entering in the bloodstream. Because of this delay, the glucuronidation of DHA to DHA-Glucuronide is slowed, as shown by a Cₘₐₓ of 60 minutes for Artesunate/Starch Glycolate compared with a Cₘₐₓ 15 minutes for untreated Artesunate (FIG. 20, bottom). Also, the intensity of the glucuronidation observed for Artesunate/SG is lower, which explains why its bioavailability is higher than for the untreated Artesunate.

TABLE 3
Artesunate pharmacokinetic parameters in mice
after receiving intravenously a dose 3.5 mg/kg

| | Groups | |
|---|---|---|
| | Untreated | Artesunate/Starch |
| Parameter | Artesunate | Glycolate |
| Route of Administration | intravenous | intravenous |
| Dose (mg/kg) | 3.5 | 3.5 |
| Cₘₐₓ | 0.06 | 0.09 |
| Maximum plasma concentration | | |
| (nmol/mL) | | |
| Tₘₐₓ | 15 | 15 |
| Maximum peak time (min) | | |
| T½ | 12 ± 2 | 14 ± 2 |
| Apparent plasma half-life (min) | | |
| Cl | 6930 ± 398 | 4821 ± 1054 |
| Clearance (mL/min/kg) | | |
| Vz | 44683 ± 14804 | 86869 ± 33326 |
| Apparent volume of | | |
| distribution (mL/kg) | | |
| AUC | 507 ± 102 | 727 ± 159 |
| Area under curve (min · ng/mL) | | |

TABLE 4
DHA pharmacokinetic parameters in mice
after receiving orally a dose 200 mg/kg

| | Groups | |
|---|---|---|
| | Untreated | Artesunate/Starch |
| Parameter | Artesunate | Glycolate |
| Route of Administration | Oral | Oral |
| Dose (mg/kg) | 200 | 200 |
| Cₘₐₓ | 0.38 | 0.50 |
| Maximum plasma concentration | | |
| (nmol/mL) | | |
| Tₘₐₓ | 15 | 15 |
| Maximun peak time (min) | | |
| t½ | 18 ± 2 | 22 ± 1 |
| Apparent plasma half-life (min) | | |
| Cl | 24295 ± 8527 | 34585 ± 5448 |
| Clearance (mL/min/kg) | | |
| Vz | 755399 ± 332498 | 895526 ± 182274 |
| Apparent volume of | | |
| distribution (mL/kg) | | |
| AUC | 2232 ± 2889 | 5783 ± 911 |
| Area under curve (min · ng/mL) | | |
| Oral Bioavailability F (%) | 8 ± 4 | 15 ± 4 |

### Tissues Distribution Profiles

Tissue distribution studies are performed, which may be helpful in providing possible relationships between plasma levels, drug levels in tissues and toxicity. For all formulations administrated intravenously, higher concentrations of active metabolite DHA are detected in brain and lower levels are observed in liver and prostate. No significant difference of DHA concentrations between these two organs are observed.

For all formulations administrated orally, higher levels of active metabolite drug are found in liver and prostate after administration. The lowest concentration is observed in the brain. Furthermore, in comparison to the artesunate/SG formulation, the untreated artesunate displays significantly lower values in all results obtained in the present experiment.

### Biodistribution in the Brain

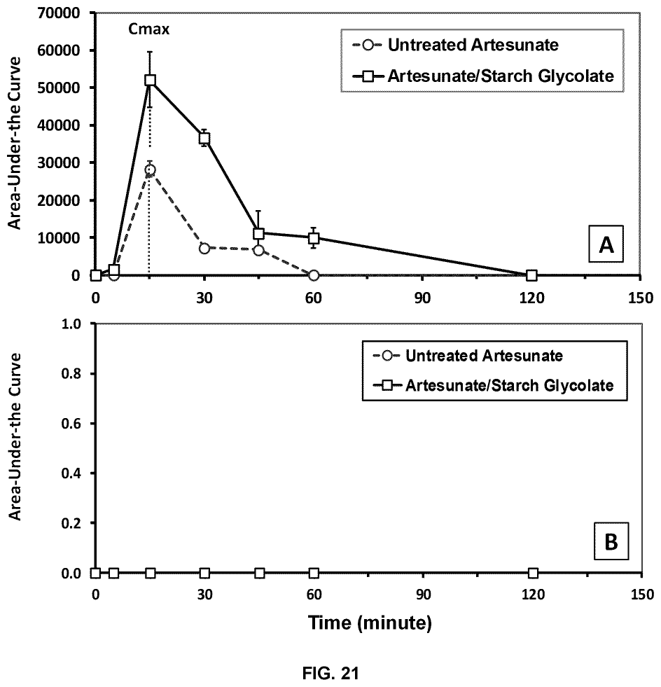

Now referring to FIG. 21, after intravenous administration, an important quantity of DHA is detected after 15 minutes (Tₘₐₓ) in the brain for the artesunate/SG formulation and its area-under-the curve (AUC) is approximately 2 times higher compared with untreated artesunate. No DHA-glucuronide is detected in the brain.

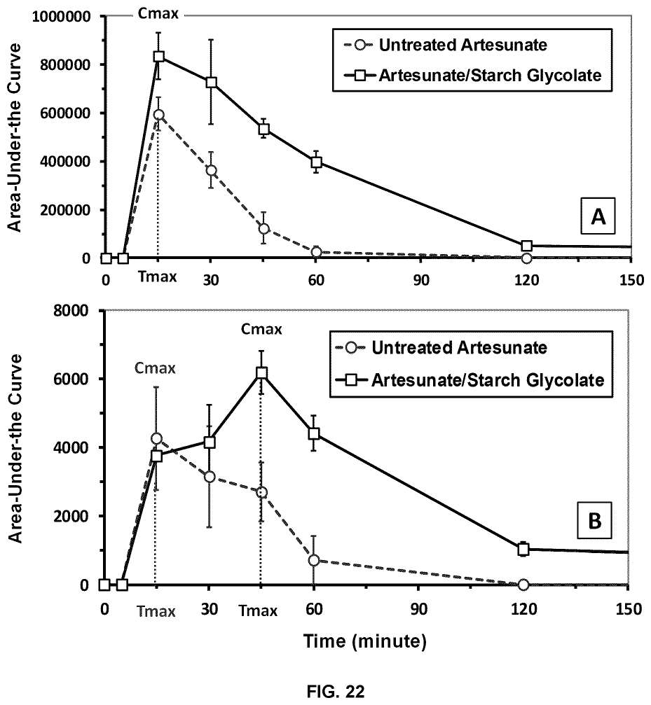

Now referring to FIG. 22, for oral administration, similar results are obtained. The maximum concentration for artesunate/SG formulation is reached after 15 minutes in the brain. In contrast for intravenous administration, the inactive metabolite DHA-glucuronide is detectable in the brain. Although DHA-glucuronide is not detected in the brain by intravenous administration, a significant amount has been observed from oral administration. This is possibly because the intravenously administrated dose (3.5 mg/kg) is lower than the oral dose (200 mg/kg). Concerning the conversion of DHA to DHA-Glucuronate, the glucuronidation is delayed for artesunate/SG formulation and the Tₘₐₓ is observed at 60 minutes whereas glucuronidation is faster for the untreated artesunate and observed at 15 minutes. This difference can explain why the apparent half-life of the artesunate/SG formulation is unexpectedly superior to untreated artesunate by intravenous and oral administration.

### Biodistribution in the Liver

Hepatic first pass occurs when drug absorbed from the gastrointestinal tract is metabolized by enzymes within the liver to such an extent that most of the drugs does not exit the liver and, therefore, does not reach the systemic circulation. To bypass these absorption barriers, intravenous administration can directly deliver drugs in organs.

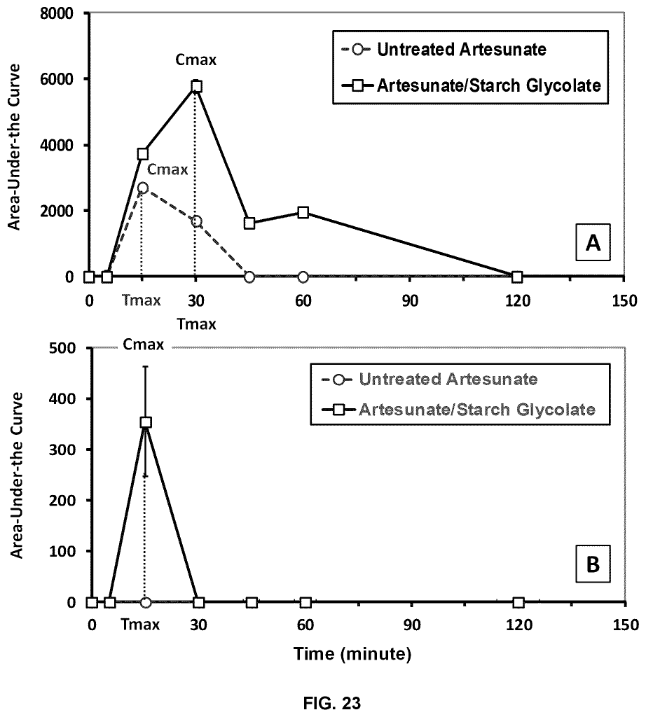

For intravenous administration (FIG. 23), artesunate is directly passed in the blood stream and hydrolyzed to DHA. Results show that the Cₘₐₓ of DHA is only detected after 30 minutes in liver, instead after 15 minutes, as observed for the oral administration which the Artesunate should first undergo the biotransformation. For this reason, lower concentration of DHA in the liver is observed for intravenous administration.

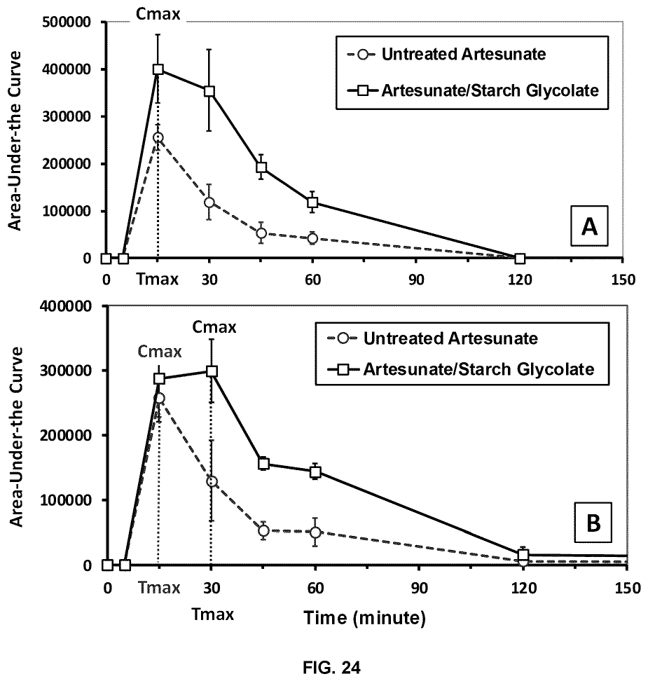

For oral administration, results (FIG. 24) clearly suggest that there is accumulation in the liver with important values that is approximately 70 times higher than by the intravenous administration. This phenomenon is coherent, because artesunate administered intravenously is directly in the bloodstream and reaches the liver later. In contrast, Artesunate administrated orally is substantially passed through the liver to undergo the biotransformation before entering in the bloodstream, which explains its higher concentration in this organ.

In general, the artesunate/SG formulation presents a higher bioavailability and a longer half-life which are probably due to the entrapment of artesunate in the starch glycolate. In fact, starch glycolate plays numerous important roles: i) starch glycolate protects artesunate whereas untreated artesunate is hydrolyzed during the transit in gastric acid; ii) delay caused by hydrolysis of starch glycolate to release artesunate before absorption and biotransformation in the liver; iii) starch glycolate can prolong the transit of active metabolite DHA in the bloodstream and delay the conversion of DHA in DHA-glucuronidation when intravenously administered.

### Biodistribution in the Prostate

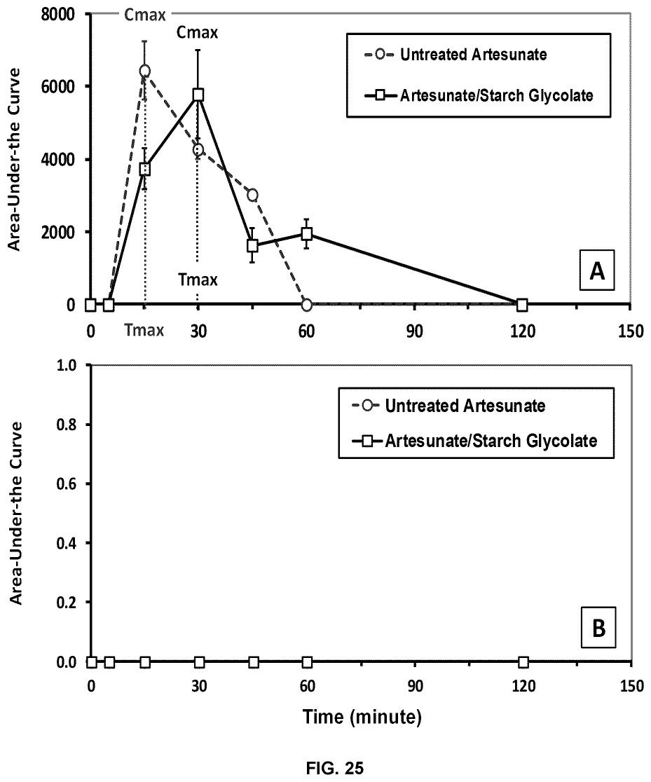

After intravenous administration, similar area-under-the curve (AUC) for both formulations is observed. However, DHA in the prostate is detected after 15 minutes for untreated Artesunate and after 30 minutes for the artesunate/SG formulation. No inactive metabolite DHA-glucuronide is detected in the prostate from intravenous administration. See FIG. 25.

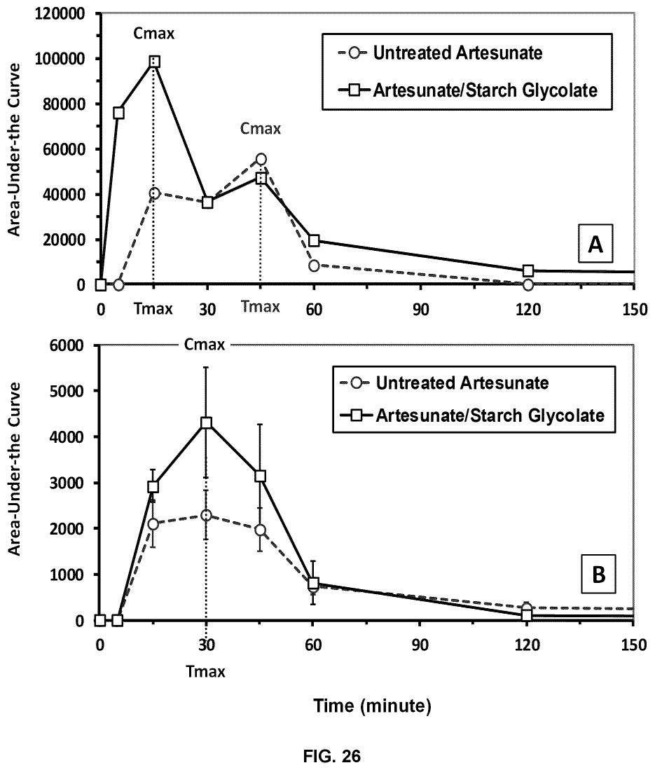

For oral administration (FIG. 26), similar profiles are obtained. However, the maximum concentration of DHA in the brain for the artesunate/SG formulation is reached after 15 minutes whereas that for untreated artesunate is reached after 30 minutes.

The inactive metabolite DHA-glucuronide for both formulations is detectable in the prostate for oral administration. Although DHA-glucuronide is not detected in the prostate by intravenous administration, a significant amount has been observed for oral administration. This may be due to the administrated intravenous dose (3.5 mg/kg) is too low to detect compared to the oral dose (200 mg/kg). The DHA-Glucuronate formation, the glucuronidation reaction is slower for artesunate/SG complex and the Cₘₐₓ is observed at 60 minutes. However, this processing is faster for the untreated artesunate and the Tₘₐₓ is observed at 15 minutes. This difference can explain why the higher bioavailability of artesunate/SG complex in the prostate.

### EXAMPLE 7

### Pharmacokinetic Profile of Water-Soluble Artesunate Compared with Commercial Artesunate

The purpose of this study is to evaluate the pharmacokinetics of Water-Soluble artesunate vs commercial (untreated) artesunate when administered by oral gavage to albino rats.

The protocol and procedures involving the care and use of animals in this study are reviewed and approved by CR-MTL Institutional Animal Care and Use Committee (IACUC) before conduct. During the study, the care and use of animals are conducted in accordance with the guidelines of the USA National Research Council and the Canadian Council on Animal Care (CCAC). 21 male Sprague-Dawley rats (age 4-8 weeks purchased from Charles River Laboratories, St-Constant) are acclimation for one week under condition as follows: Temperature: 19° C. to 25° C.; Humidity: 30% to 70%; Light Cycle: 12 hours light and 12 hours dark. All rats are fed ad libitum throughout the study, except during designated procedures. The feed is analyzed by the supplier for nutritional components and environmental contaminants. Results of the analysis are provided by the supplier and are on file at the Testing Facility. There are no known contaminants in the feed that would interfere with the objectives of the study.

TABLE 5
EXPERIMENTAL DESIGN

| | | | Dose | | Formulation | |
|---|---|---|---|---|---|---|
| | | Dosing | Level | Dose vol | conc | Group |
| Group | Treatment | route | (mg/kg) | (mL/kg) | (mg/mL) | size |
| 1 | Untreated Artesunate | PO | 200 | 10 | 20.00 ^{a} | 21 |
| 2 | Water-soluble Artesunate | PO | 200 | 20 | 16.66 ^{b} | 21 |

PO: Oral gavage;
^{a} solvent = absolute ethanol;
^{b} solvent = distilled water

Blood is collected (jugular vein) from all animals at specified time as detailed below. At the end of experience (120 min), blood samples are collected through abdominal aorta or vena cava.

TABLE 6
Sample Collection

| | Time post dose on Day 1 | | | | | | |
|---|---|---|---|---|---|---|---|
| Group | 5 min | 15 min | 30 min | 45 min | 60 min | 120 min | 240 min |
| 1 and 2 | X | X | X | X | X | X | X |

| | |
|---|---|
| Method/Comments: | Jugular venipuncture |
| | Terminal via abdominal aorta |
| Target Volume (mL): | 0.4 mL; Jugular vein |
| | 1 mL; terminal |
| Anticoagulant: | K₂EDTA |
| Processing: | Plasma |

X = Sample to be collected; min = minutes.

After collection, blood samples in K₂EDTA tubes are placed on wet ice and centrifuged as per internal settings. Plasma (150 μL) is placed on dry ice and transferred to a freezer set to maintain at −80° C. for PK bioanalysis.

### Sample Extraction

A volume of 10 μL of sample was extracted by adding 60 μL of Acetonitrile containing IS. After vortexed briefly and centrifuged for 5 min at 3000 rpm, a volume of 50 μL of supernatant is transferred into a clean plate and diluted with 50 μL of Milli-Q water. The standards are prepared in pre-quenched matrix to be precautious for Ester stability.

On the day of analysis, plasma samples will be mixed with 1mL of acetonitrile and centrifuged for analysis by LC-MS/MS (2 analytes). Non-compartmental analysis of the test item analytes (2) in blood matrix concentrations will be performed using validated computer software Phoenix for each dose level, gender, and occasion (1) following standard operating procedures. For LC-MS/MS analysis, parameters are represented in the Table 7 below.

TABLE 7
LC-MS/MS parameter analysis
Sample Analysis

| Assay type | RGA Level 1 (±25%, LLOQ ±30%) | | | |
|---|---|---|---|---|
| LC Conditions | | | | |
| Column id. & Dimensions | Macmod; ACE 3 C18-AR; 30 × 2.1 mm | Time (sec) | % MPB | Flow (mL/min) |
| Temperature (° C.) | 45 | 15 (step) | 50 | 0.800 |
| Mobile Phase A | 0.1% Formic Acid in 95:5 Water:Acetonitrile | 60 (Ramp) | 90 | 0.800 |
| Mobile Phase B | 0.1% Formic Acid in 50:50 Water:MeOH | 5 (Ramp) | 95 | 0.800 |
| Needle Rinse 1 | 50:25:25 Isopropyl Alcohol:Acetone:Acetonitrile | 30 (step) | 95 | 0.800 |
| Needle Rinse 2 | 10% MeOH:90% H₂O:0.1% Formic Acid | 40 (step) | 50 | 0.800 |

| MS Conditions | | | |
|---|---|---|---|
| MS/MS | API5500 | | |
| Ionization method | Electrospray | | |
| Positive/Negative Ion | Positive ion | | |
| Resolution | Unit/Unit | | |
| Source Temperature (° C.) | 550 | | |
| Transition (m/z) | | | |
| Analyte(s) | Compound Id: | Artesunate | 407.200/261.200 Da |
| ISTD(s) | ISTD Id. | Glyburide | 494.200/369.100 Da |

Results

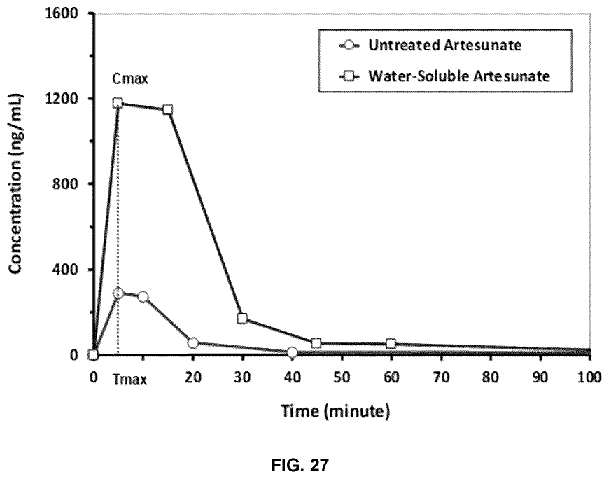

Now referring to FIG. 27, the results show that there is significant difference between commercial Artesunate and Water-Soluble Artesunate after oral administration. The maximum average concentration (Cₘₐₓ) reached after 10 minutes (Tₘₐₓ) is respectively 290 and 1178 ng/mL for untreated Artesunate and Water-Soluble Artesunate. Furthermore, the area under the curve (AUC) of Water-Soluble Artesunate is larger than AUC of commercial Artesunate suggested that the bioavailability of Water-Soluble Artesunate is greater than commercial Artesunate (approximately 4.0 times).

### EXAMPLE 8

### In Vivo Test for Evaluation of Anti-Helicobacter Pylori Activities Animals

C57BL/6 mice with SPF («specific pathogen free») status, were housed at the A2 pet store at the University of Bordeaux, in an acclimatization room for 1 week and then transferred to zone of experimentation where the project was conducted.

### Gavage by Helicobacter Pylori

The H. pylori PremSS1 strain (supplied by Dr Anne Mueller, University of Zurich) was used. The functionality of the cagPAl island was verified in vitro in a coculture model with the gastric epithelial line AGS: induction of a so-called «hummingbird» phenotype.

The mice were force-fed at 6 weeks of age with the H. pylori premSS1 strain. The gavages were carried out 3 consecutive days, with a rich suspension of bacteria (2 to 3 dishes of rich culture for 5 mice). PremSS1 was poured on agar called «Pylo house made» prepared in the laboratory (Wilkins Chalgren medium enriched with 10% human blood and made selective by the addition of vancomycin 10 pg/ml, trimethoprim 5 μg/ml, amphotericin B 1 μg/ml and Cefsulodin 2μg/ml). PremSS1 was collected after 24 hours in incubation in a microaerobic atmosphere at 37° C. H. pylori was identified by its phenotypic and biochemical characteristics (morphology, urease test, oxidase test) before harvesting.

Indeed, the mice were fasted the day before the gavage days. They were force-fed in the morning with 100 μl of bacterial suspension and then placed in a cage for the rest of the day under normal conditions. In the evening, they are transferred in a cage with clean litter in order to prevent them from eating their excrements during nightly fasting. This protocol was repeated for the 3 days of force-feeding. A bacteria viability test was carried out post-gavage by platting a bacterial pellet on «Pylo house made» agar for 24 hours.

### Preparation of Natural Compounds Before Treatment

Different tested compounds are administered to mice (6 weeks old) after infection with H. pylori for 7 to 14 consecutive days.

An average weight is established for each group. For each solution, the double volume required has been prepared. All solutions were prepared extemporaneously daily during the 7 to 14 days of treatment.

All necessary quantities of compounds were weighed separately using a precision balance under sterile conditions and then dissolved in sterile distilled water. The tubes are then wrapped in aluminum foil to avoid exposure to light.

### Preparation of Pantoprazole and Amoxicillin

A quantity necessary for the number of mice treated by Pantoprazole (Arrow Lab Generic) is prepared at a dose of 150 mg/kg and for a volume of 100 μL/mouse in sterile distilled water. After completely dissolved pantoprazole, an amount of amoxicillin (Amox from Panpharma Laboratory) 30 mg/kg is added.

### Preparation of the Compounds NACINN and Artemisinin/SG

A necessary quantity of artemisinin/SG or of NACINN depending on the number of mice to be treated is weighed and then dissolved either in water or in the pantoprazole solution and then brought to 37° C. under stirring for 15 minutes minimum and protected from light. Once the solution was well homogenized, an aliquot is withdrawn in sterile 2 mL Eppendorf tubes.

### Conducting of the Experience

The first experiment planned to treat for 7 days, three weeks post-oral gavage with H. pylori, then to sacrifice half of the mice of each group in order to quickly determine the antibacterial effect of the compounds: cinnamaldehyde (Nacinn), artesunate/SG (Arte/SG) complex or water-soluble artesunate (WSA) alone or in combination with amoxicillin. Proton-Pump inhibitors (PPI) and amoxicilline (Amox) alone are also included in this study.

### Sacrifice

The mice were sacrificed by cervical dislocation and opened by laparotomy the day after the last day of treatment. The stomach was isolated and removed by cutting close to the esophagus and the duodenum. It is opened by the large curvature with a small curved end scissor and put in a petri dish with a little physiological saline in order to remove the food that was inside.

The stomach was then cut in 2 along the axis of the large curvature, from the duodenum to the esophagus, and then cut in 2 along the axis of the small curvature. The right half-stomach cleared of cardia was cut in 2, the first half introduced into a tube with physiological saline (for bacteriological culture and molecular study) and the second half put in a dry tube to be stored at −80° C. (complementary experiences). The left half-stomach was placed in a tube containing formalin for fixation (histology of inflammation analysis).

### Quantitative Culture of H. Pylori

Each quarter of the mouse stomach is collected in a tube (RNASE free, DNASE free) containing 200 μL of physiological water. The tube containing the stomach piece was weighed to determine the weight of the stomach fragment. The stomach fragment was then ground using a sterile pestle. An amount of 10 μL are spread on a whole box of GSSA agar prepared in the laboratory (Wilkins Chalgren medium enriched with 10% human blood and made selective by the addition of vancomycin, trimethoprim, amphotericin B, nalidixic acid, bacitracin, polymyxin B and Cefsulodine), using 100 μL of physiological water previously deposited in the middle of the box. 100 μL of dilution were then used to inoculate the following dishes of GSSA medium: 2 dishes with a dilution of 10⁻¹, 2 dishes with a dilution of 10⁻², and 2 boxes with a dilution of 10⁻³.

The 90 μL remaining were extracted for the molecular biology part (see below in the section «Relative Quantification of H. Pylori by qPCR»).

Only the 10 μL pure crushed stomach was spread for the 10 uninfected mice.

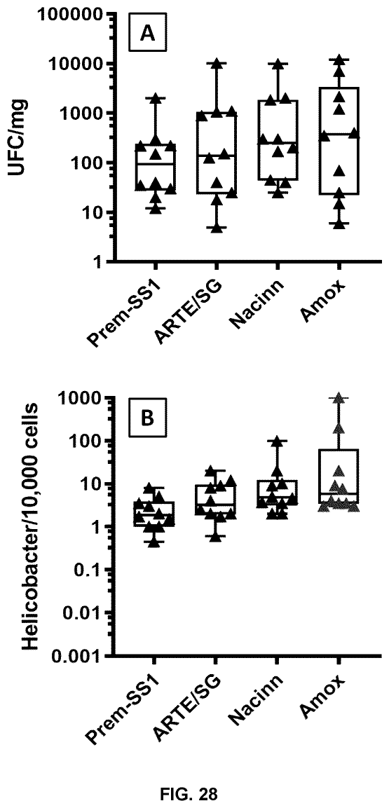

The dishes were incubated at 35° C. in a microaerobic atmosphere and H. pylori was identified by its phenotypic and biochemical characteristics (morphology, urease test, oxidase test). The dishes were read after at least 5 days of incubation. A colony count was carried out by two independent experimenters. The results are expressed in CFU/mg of stomach. See FIG. 28.

### Relative Quantification of H. Pylori by Quantitative PCR

Automated extraction: after seeding part of the crushed stomachs, the rest of the stomachs (90 μl) were used to extract the total DNA with the MagNa Pure 96 extractor (Roche Diagnostics™) using the MagNA Pure 96 DNA kit and Viral NA Small Volume Kit, and following the manufacturer's recommendations. For each ground material, the extracted DNA was recovered in 100 μl of elution buffer.

Quantitative PCR: the presence of H. pylori DNA and mouse housekeeping genes is quantified in the extracts by quantitative PCR in real time, by detecting the fluorescence emitted by the neo PCR products formed using Sybr Green™. This determines the number of cycles from which the PCR product is detectable, called the threshold cycle.

The specific amplification of H. pylori is carried out using a pair of primers targeting the gene coding for 23S rRNA, present in two copies in H. pylori. A 267-bp fragment of the 23S rRNA gene of H. pylori is amplified by using primers HPY-S and HPY-A. The primers are analyzed for 3′-terminal specificity to assure that they were specific to H. pylori. The sequences of these primers are HPY-S (AGGTTAAGAGGATGCGTCAGTC) (DEQ ID NO:5) and HPY-A (CGCATGATATTCCCATTAGCAGT) (SEQ ID NO:6. These sequences correspond to nucleotides 1931 to 1952 and 2197 to 2175, respectively, of the 23S rRNA gene of H. pylori (Gen Bank accession number U27270).

The quantification of H. pylori is normalized via the mouse reference genes Gapdh and Beta actin. The primers used are mGapdh1 for (CTGCAGGTTCTCCACACCTATG) (SEQ ID NO:1), mGapdh1rev (GAATTTGCCGTGAGTGGAGTC) (SEQ ID NO:2), mActb2for (GACAGGATGCAGAAGGAGATTACTG) (SEQ ID NO:3) and mActb2 rev (ACATCTGCTGGAAGGTGGACA) (SEQ ID NO:4). All were designed with the help of Express Technologies.

A volume of 5 μL of DNA is added to 20 μL of reaction mixture (primers 0.4 μM, Master Mix 2× supplied by the «LightCycler0 480 SybrGreen I Master» kit from Roche Diagnostics®), at 20 ng/μL to amplify the gene coding for 23S rRNA and at 2 ng/pL to amplify the reference genes. The amplification in the LC480® from Roche Diagnostics® took place according to the following program below:

TABLE 8
Light cycle amplification program

| | Temperature | Duration | Transition | |
|---|---|---|---|---|
| | (° C.) | (minute) | (° C./s) | Mode |
| Denaturation | 95 | 10 | 4.40 | — |
| Quantification 45 | 95 | 0 | 4.40 | — |
| cycles | 60 | 10 | 2.20 | — |
| | 72 | 15 | 4.40 | Single |
| Fusion | 95 | 1 | 4.40 | — |
| | 38 | 50 | 2.20 | — |
| | 90 | / | 0.11 | Continue |
| Cooling | 40 | 30 | 2.20 | — |

Two standard curves indicate the number of murine cells per microliter of DNA and the number of bacteria per microliter of DNA are created in order to quantify the number of murine cells (MSCR murine line) and bacteria in the sample. For each mouse, the result of the quantification is then calculated by obtaining the ratio of the number of bacteria per microliter of DNA (bacteria number/μL of DNA) as a function of the number of murine cells per microliter of DNA (murine cell/pL of DNA). Each mouse DNA is analyzed in duplicate.

Two standard curves showing the number of murine cells per pl of DNA and the number of bacteria per pl of DNA were performed in order to quantify the number of murine cells (murine line MSCR) and bacteria in the sample. For each mouse, the quantification result is then calculated by obtaining the ratio of the number of bacteria/μl of DNA as a function of the number of murine cells/μl of DNA. Each mouse DNA is analyzed in duplicate.

### Experiment 1

Study the antibacterial activity of different components in mice in the absence of Proton Pump Inhibitor
Group-0: mice not infected (NI), n=10
Group-1: mice infected with pre-mSS1, n=10
Group-2: mice infected with pre-mSS1+ARTE/SG (40 mg/kg), n=10
Group-3: mice infected with pre-mSS1 +Nacinn (40 mg/kg), n=10
Group-4: mice infected with pre-mSS1+Amoxicillin (Amox, 30 mg/kg, n=10

### Results Of Experiment 1

Now referring to FIG. 28, after 7 days of treatment in absence of proton pump inhibitor, no antibacterial effect is detected by quantitative culture or quantitative PCR for all tested compounds, including Amoxicillin.

### Experiment 2

Study the antibacterial activity of different components in mice in the presence of Proton Pump Inhibitor.

In the present study, the duration of treatment is for 14 days and Pantoprazole (PPI) is added in the combination formulation at dose 150 mg/kg. The groups were therefore as follows:

Group-0: mice not infected
Group-1: mice infected with pre-mSS1 +Pantoprazole (150 mg/kg), n=10
Group-2: mice infected with pre-mSS1 +ARTE/SG (40 mg/kg)+Pantoprazole (150 mg/kg), n=10
Group-3: mice infected with pre-mSS1 +Nacinn (40 mg/kg)+Pantoprazole (150 mg/kg), n=10
Group-4: mice infected with pre-mSS1 +Amoxicillin (Amox, 30 mg/kg +Pantoprazole (150 mg/kg), n=10

### Results of Experiment 2

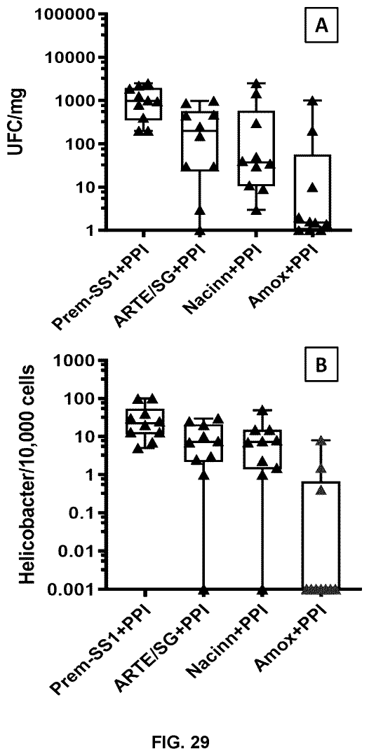

Now referring to FIG. 29. After 14 days of treatment in the presence of proton pump inhibitor, there are certain antibacterial effect that are detected for Amoxicillin (30 mg/kg) by quantitative culture or quantitative PCR. However, a slight antibacterial effect is observed for Artesunate/SG complex and Nacinn (Cinnamaldehyde), but they are not significant (P value <0.05 Mann Whitney test between untreated infected mice and different experimental conditions) for the dose 40 mg/Kg. The box plots rectangle represent 50% of the values around the median and the segments at the ends showing the minimum and maximum of all the data.

### Experiment 3

Study the antibacterial activity of different components at high dose in the presence of Proton Pump Inhibitor.

In this study, the doses of ARTE/SG and NACINN have been increased. For artesunate/starch glycolate (Arte/SG), the concentration is increased from 40 to 200 mg/kg (5 times) and for Nacinn, from 40 to 120 mg/kg (3 times)
Group-1: mice not infected (NI)+n=10
Group-2: mice infected with pre-mSS1+Amoxicillin (Amox, 30 mg/kg)+Pantoprazole (PPI, 150 mg/kg), n=10
Group-3: mice infected with pre-mSS1+Nacinn (120 mg/kg)+Pantoprazole (PPI, 150 mg/kg), n=10
Group-4: mice infected with pre-mSS1+Nacinn (120 mg/kg)+Amoxicillin (Amox, 30 mg/kg)+Pantoprazole (PPI, 150 mg/kg), n =10
Group-5: mice infected with pre-mSS1+Arte/SG (200 mg/kg)+Pantoprazole (PPI, 150 mg/kg), n =10

Group-6: mice infected with pre-mSS1+Arte/SG (200 mg/kg)+Amoxicillin (Amox, 30 mg/kg)+Pantoprazole (PPI, 150 mg/kg), n=10

### Results Of Experiment 3

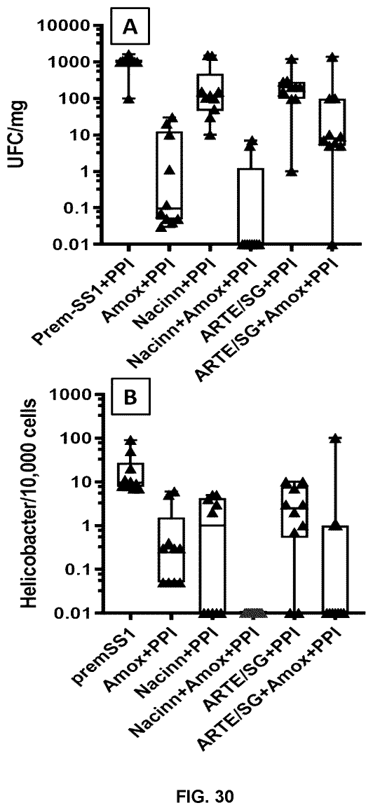

Now referring to FIG. 30. After 14 days of treatment in the presence of proton pump inhibitor, results show that no antibacterial activity for all compounds administrated alone is observed. Similar results are observed for combination formulation of artesunate/SG and Amoxicillin. However, the combination of Nacinn (Cinnamaldehyde at 120 mg/kg) with Amoxicillin (30 mg/kg) in present of Pantoprazole (150 mg/kg) show a significant antibacterial activity. These results are confirmed by quantitative PCR tests.

It is important to mention that the maximum volume of liquid permitted to administer by gavage in mice is very limited (about 100 μL), it is possible that artesunate/SG complex (200 mg/kg) is not completely solubilized in the suspension explaining why its failed. To overcome this barrier, water-soluble artesunate is developed.

### Experiment 4

Study the antibacterial activity of water-soluble artesunate in the presence of Proton Pump Inhibitor.

As mentioned previously, water-soluble artesunate (WSA) possesses an antibacterial activity superior to untreated artesunate or other treatments including artesunate entrapped in starch glycolate (Artesunate/SG complex). This is believed to be mainly due to the high solubility and stability of WSA in solution.

For this reason, the present experiment is to highlight and to confirm the in vitro study. All the conditions are similar with the previous experiment, except the artesunate/SG is replace by water-soluble artesunate. The dose of WSA administrated is 40 mg/kg which is 5 times smaller than that of artesunate/SG complex (200 mg/kg).
Group-1: mice not infected (NI)
Group-2: mice infected with pre-mSS1+Water-soluble Artesunate (WSA, 40 mg/kg)
Group-3: mice infected with pre-mSS1+Water-soluble Artesunate (WSA, 40 mg/kg)+Pantoprazole (PPI, 150 mg/kg)
Group-4: mice infected with pre-mSS1+Water-soluble Artesunate (WSA, 40 mg/kg)+Amoxicillin (Amox, 30 mg/kg)+Pantoprazole (PPI, 150 mg/kg), n=10

### Results Of Experiment 4

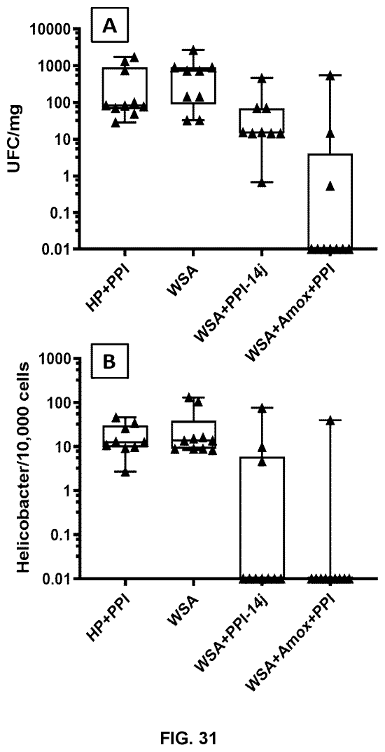

Now referring to FIG. 31, similar to results obtained previously, no significant antibacterial activity for water-soluble artesunate (WSA, 40 mg/kg) is detected in the absence of Proton Pump Inhibitor. In contrast, in the presence of Proton Pump Inhibitor, there is a significant difference, particularly when combined with Amoxicillin. These results are confirmed by quantitative culture and by quantitative PCR.

In each of FIGS. 28 to 31, the graphical representations are box plots, the rectangle representing 50% of the values around the median and the segments at the ends showing the minimum and the maximum of all the data. *P <0.05, **P <0.01 and ***P <0.001 between untreated infected mice and different experimental conditions (Mann Whitney test).

These data demonstrate the unexpected advantage of combining NACINN (Cinnamaldehyde) and Water-Soluble Artesunate and (with an IPP to obtain an antibacterial effect in vivo). Furthermore, the combination of artesunate or cinnamaldehyde with amoxicillin also permit to obtain unexpectedly good results.

### EXAMPLE 9

### In Vitro Tests of Cinnamaldehyde/Starch Glycolate and Artesunate/Starch Glycolate Complexes to Treat Leukemia

In this leukemia study, ARTE means native (untreated, unencapsulated) artesunate and ARTE.SG (artesunate.Starch Glycolate) means artesunate/Starch Glycolate complex.

### Methodology

Cell Growth Measurement

Cell growth was followed via the resazurin (alamar blue) fluorescence assay. At day −1, cells were seeded in 100 μL media at a density of 4×10³ cells/well in 96-well plates and incubated overnight at 37° C. At day 0, cells were exposed to either 2 μM or various concentrations of artesunate or artesunate/Starch Glycolate complex at a final volume of 200 μL. Cultures were performed during 3 days after the addition of the drugs. At day 3, Resazurin (0.1 mg/mL, Sigma-Aldrich) was added at 20 μL/well, and plates were incubated for 4 h at 37° C., then fluorescence (Aex=529.5 nm, Aem =582 nm) was measured using ClarioStar™ microplate reader. In each experiment, all wells contained the same amount of methanol and H₂O used as solvent and vehicle controls for artesunate and artesunate/Starch Glycoate complex, respectively. The amount of methanol and water used corresponded to the amount required by the maximum concentration condition for both artesunate and artesunate/Starch Glycolate complex.

### Statistical Analysis

Statistical analysis was performed in R software (v 3.3.2) using the stats package.

### Results

1. Selection of AML Cell Lines that are Highly Sensitive to Artesunate:

The aim of this test was to select AML cell lines that are highly sensitive to artesunate. Those supposedly selected cells may be used to test for artemisinin sensitivity before and after encapsulation. This first objective, in addition to checking the sensitivity to artesunate (ARTE), is to test the sensitivity to the complex form (ARTE/Starch Glycolate) as well. This provides information on whether the encapsulation process affects the efficiency of ARTE in vitro. The effects of both drugs on the proliferation of 10 AML cell lines and 1 CML cell line was tested. Both the drug or the drug/Starch Glycolate complex were used at 2 μM, a dose that has been reported to be very efficient at least in two AML cell lines (MV4-11 and MOLM13). The ARTE/Starch Glycolate in water was prepared fresh every time for each experiment. Frozen (−20° C.) ARTE/Starch Glycolate complex was also tested in parallel to fresh ARTE/Starch Glycolate in each experiment, to evaluate stability after freezing.
a. Encapsulated artesunate (artesunate/SG) is as efficient as artesunate (ARTE) in 11 leukemic tested cell lines:

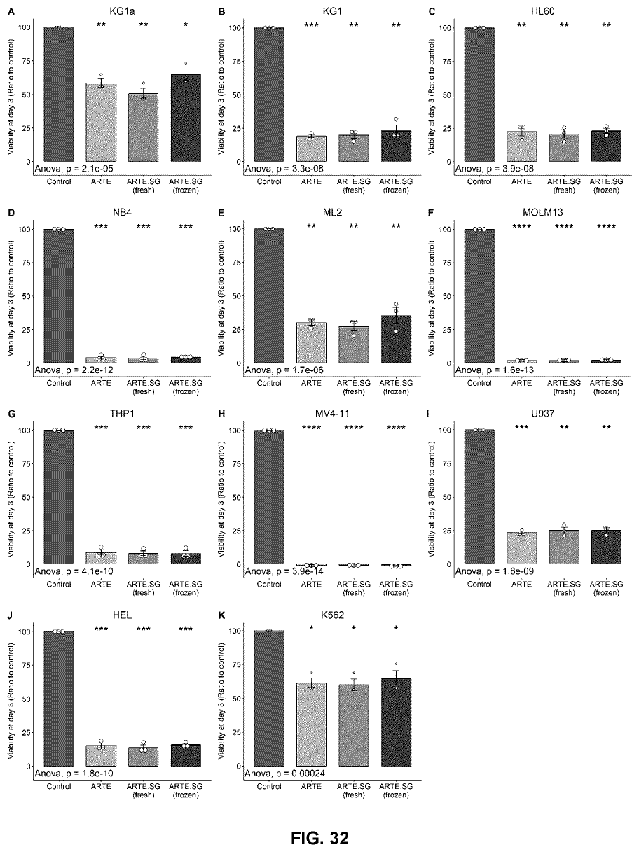

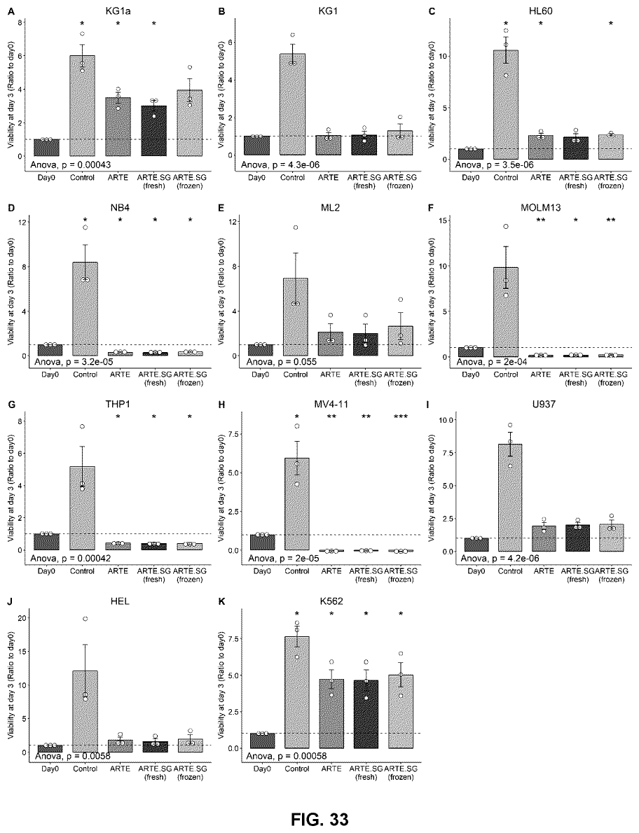

The results show that artesunate/SG (ARTE.SG) (2 μM) is as efficient as artesunate (ARTE) (2 μM) in all tested cell lines (FIG. 32). As expected, we found MV4-11 and MOLM13 cells to be highly sensitive to ARTE and reluctantly to ARTE.SG as well. In addition, THP1 and NB4 cell lines were also as sensitive; their signal at day 3 is lower than that of the control at day 0 indicating mortality effects (FIG. 33).

Three groups of cells can be globally distinguished: those that are highly sensitive to ARTE and ARTE.SG including NB4, MOLM13, MV4-11 and THP1 cells; those that are moderately sensitive including KG1, HL60, ML2, U937 and HEL cells; and those with low sensitivity including KG1a and K562 cells. The results also show that ARTE.SG stored at −20° C. was as effective as newly prepared ARTE.SG in all tested cell lines.
b. Encapsulated Artesunate (ARTE.SG) is Stable at 4° C.:

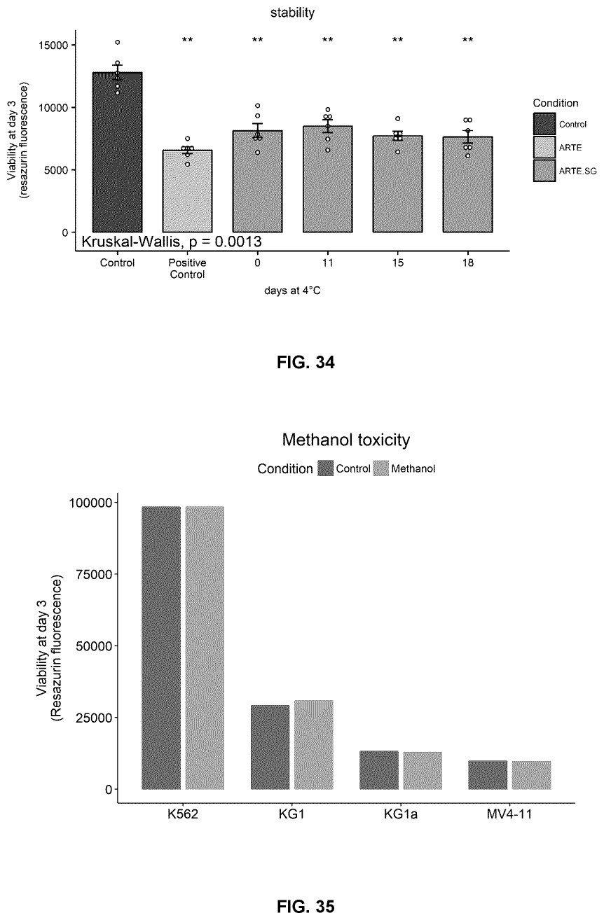

Next, the long-term stability of ARTE.SG in aqueous solution (5 mM in distilled water) was determined. It was reasoned that a decrease in ARTE.SG stability would be most apparent in a cell line with low sensitivity, therefore, the KG1a cell line was selected for this test. The results show that ARTE.SG, solubilized in water, is stable at 4° C. for at least 18 days (FIG. 34).
2. ARTE.SG and ARTE have the Same IC₅₀ in 4 Representative Leukemic Cell Lines

To further confirm the equivalent efficiency between ARTE and ARTE.SG, the IC₅₀ of both drugs on 4 leukemic cell lines that cover the 3 sensitivity groups described above was calculated. Those cell lines are: MV4-11 (highly sensitive), KG1 (moderately sensitive), KG1a (weakly sensitive) and K562 (weakly sensitive, the only cell line that represents CML).

Unlike ARTE.SG that is prepared in water (Stock at 5 mM), the stock of ARTE (20 mM) was prepared in methanol. Therefore, before proceeding to calculate the IC₅₀ for each drug, we checked the toxicity effect of methanol on our 4 cell lines. For this, a dose of methanol (0.16%) that corresponds to the highest concentration of ARTE (32 μM) used in the IC50 experiment was used. It can clearly be seen that at such dose methanol does not affect the growth of our cells under our experimental conditions (FIG. 35).

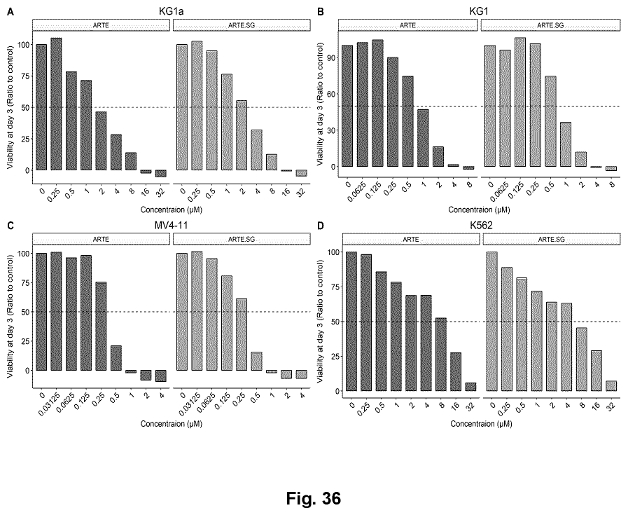

The preliminary suggest equivalent IC₅₀ for ARTE and ARTE.SG on all tested cell lines (FIG. 36). Indeed, KG1a and K562 harbored the lowest IC₅₀ around 2 and 8 μM, respectively. As expected MV4-11 cells were the most sensitive with an IC₅₀ between 0.25 and 0.5 μM, while KG1 cell line showed moderate sensitivity with IC₅₀ around 1 μM.

Taken together, these results show that ARTE.SG is as efficient as ARTE on AML cells, suggesting no decrease in its efficiency due to the encapsulation process, and that ARTE.SG (solubilized in water) is stable at −20° C. and at least for 18 days at 4° C.

### EXAMPLE 10

### Effect of Cinnamaldehyde Complexed with Starch Glycolate on the Proliferation of AML Cell Lines

### Methodology:

Cinnamaldehyde/SG conjugated complex (NACINN) preparation

For each experiment, a stock solution of NACINN was freshly prepared in water with 500 μg/ml of active ingredient (around 9 mg of powder per ml).

Cell Growth Measurement

Cell growth was followed via the resazurin (alamar blue) fluorescence assay. At day −1, cells were seeded at a density of 4×10³ cells/well in 96-well plates and incubated overnight at 37° C. At day 0, cells were exposed to the desired concentrations of NACINN at a final volume of 200 μL. Cultures were performed during 3 days after the addition of the drug. At day 3, Resazurin (0.1 mg/mL, Sigma-Aldrich) was added at 20 μL/well, and plates were incubated for 4 h at 37° C., then fluorescence (λex=529.5-19 nm, λem=582-36 nm) was measured using ClarioStar™ microplate reader. Six well-replicates were used for each condition per experiment.

### Statistical Analysis

Statistical analysis was performed in R software (v 3.3.2) using the stats package.

### Results

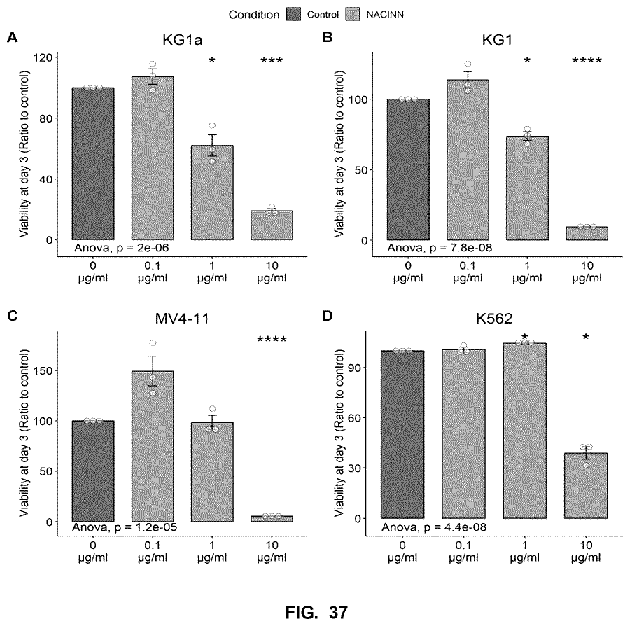

The effect of NACINN on the growth of 4 AML cell lines: KG1a, KG1, MV4-11 and K562 was tested. Since, a 10-fold serial dilution starting at 10 pg/ml is used. The IC₅₀ in the tested cell lines ranged from 1 to 10 μg/ml, which is equivalent to 7.5-75 μM (FIG. 37).

The results show that cinnamaldehyde also has anti-cancer effect on AML, but to a lesser extent than that of artesunate. To have a complete formulation against H. pylori and at the same time against stomach cancer, the two complexes could be combined.

### EXAMPLE 11

### Orally Extended Release Tablet Formulation for Water-Soluble Artesunate

TABLE 9
Formulation composition

| | Quantity (mg) | Percentage (%) |
|---|---|---|
| Water-Soluble Artesunate | 334 (corresponding | 36.34 |
| powder (containing | approx. to 200 | |
| 60% Artesunate) | mg of Artesunate) | |
| Eudragit L100-55 | 300 | 32.65 |
| Povidone ™ K12 | 150 | 16.32 |
| Hydroxypropylmethyl | 25 | 2.72 |
| Cellulose | | |
| Crospovidone | 100 | 10.88 |
| Magnesium Stearate | 10 | 1.09 |
| TOTAL | 919 | 100.0 |

In the present formulation, Eudragit is used as floating agent, Povidone as binder, Hydroxypropyl methylcellulose as controlled release agent and Crospovidone as disintegrating agent.

### In Vitro Dissolution Tests

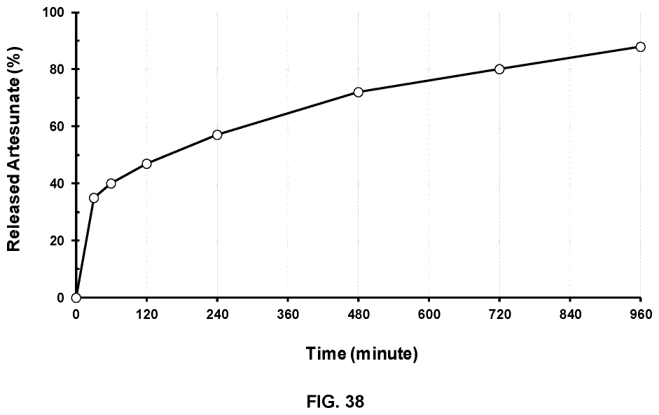

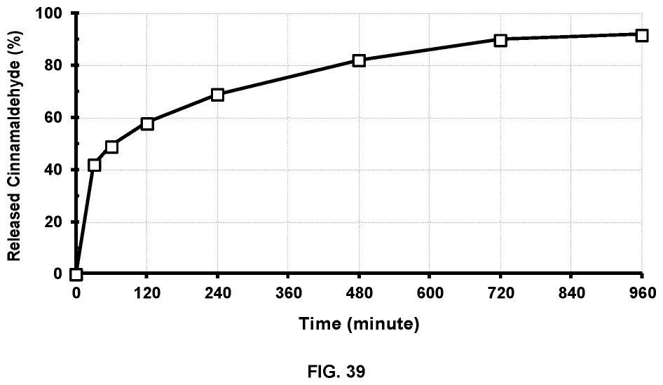

In vitro release studies of WSA monolithic tablets are carried out using an USP paddle (Apparatus 2) method with a dissolution Distek 5100 apparatus (North Brunswick, NJ, USA) at 100 rpm and 37° C. Now referring to FIG. 38, the artesunate release from monolithic tablets (n=3) in 1 L of simulated gastric fluid (SGF, pH 1.5) is spectrophotometrically measured at predetermined sampling intervals (0, 0.5, 1.0, 2.0, 4.0, 8.0, 12 and 16 h), volumes of 1 mL were withdrawn from dissolution medium to estimate the concentration of artesunate by spectrophotometry. This method, less sensitive and precise than HPLC, is chosen because it is simple and rapid. Since the maximal absorption of Eudragit is about of 265 nm, the reading for artesunate is selected at 222 nm. The release of artesunate is estimated and expressed as the relative percentage from the total loading.

The tablet immediately floated after 5-10 minutes when they entered in contact with SGF. The kinetic release profiles of Artesunate shows that there is a fast release of about 32% of artesunate (corresponding approximately to the 64 mg of Artesunate), after 30 min at 37° C. A sustained release is followed for remaining Artesunate which is achieved after 10-12 h.

### EXAMPLE 12

### Orally Extended Release Tablet Formulation for Cinnamaldehyde/Starch Glycolate Complex

TABLE 10
Formulation composition

| | Quantity (mg) | Percentage (%) |
|---|---|---|
| Cinnamaldehyde/Starch Glycolate | 600 (approx. | 61.86 |
| Complex Powders (20% | to 120 mg of | |
| Cinnamaldehyde) | cinnamaldehyde) | |
| Crospovidone | 100 | 10.31 |
| Hydroxypropylmethyl | 50 | 5.16 |
| Cellulose | | |
| Polyethylene glycol 3350 | 200 | 20.61 |
| Magnesium Stearate | 20 | 2.06 |
| TOTAL | 970 | 100.0 |

In Vitro Dissolution Tests for Cinnamaldehyde

In vitro release study of cinnamaldehyde monolithic tablets is carried out using the similar method mentioned previously. The cinnamaldehyde release from monolithic tablets (n=3) in 1 L of simulated gastric fluid (SGF, pH 1.5) is estimated by spectrophotometry after added (in excess) 1 mL of isoniazid (2 mM) solution in 1 mL of tested sample. In SGF, isoniazid reacts with trans-cinnamaldehyde (equimolar 1:1) to form a derivative absorbing maximally at 340 nm. In fact, the tablet floated after approx. 0.5-1 h in contact with SGF. Now referring to FIG. , the kinetic release profiles of cinnamaldehyde shows that there is a fast release about of 40% (corresponding to 48 mg) after 30 minutes followed a sustained release of cinnamaldehyde for a longue period more than 12 h in SGF, at 37° C.

### SEQUENCES

| SEQUENCE | | |
|---|---|---|
| ID | SEQUENCE | DESCRIPTION |
| SEQ ID | CTGCAGGTTCTCCACACCTATG | mGapdh1for |
| NO: 1 | | |
| SEQ ID | GAATTTGCCGTGAGTGGAGTC | mGapdh1rev |
| NO: 2 | | |
| SEQ ID | GACAGGATGCAGAAGGAGATTACTG | mActb2for |
| NO: 3 | | |
| SEQ ID | ACATCTGCTGGAAGGTGGACA | mActb2rev |
| NO: 4 | | |
| SEQ ID | AGGTTAAGAGGATGCGTCAGTC | 23 S-rRNA - |
| NO: 5 | | HPY-S |
| SEQ ID | CGCATGATATTCCCATTAGCAGT | 23 S-rRNA - |
| NO: 6 | | HPY-A |

While preferred embodiments have been described above and illustrated in the accompanying drawings, it will be evident to those skilled in the art that modifications may be made without departing from this disclosure. Such modifications are considered as possible variants comprised in the scope of the disclosure.

1.-10. (canceled)

11. An extended release gastro retentive dosage form comprising:
an artesunate emulsion having a pH value of from about 7.5 to 7.9 and comprising an artesunate or pharmaceutically acceptable salts thereof, and stereoisomers thereof stabilized with an emulsifying agent.

12. The extended release gastro retentive dosage form of claim 11, wherein a weight ratio of said artesunate pharmaceutically acceptable salt and said emulsifying agent in said artesunate emulsion is from about 9:1 to about 1:1.

13. The extended release gastro retentive dosage form of claim 12, wherein said weight ratio is 7:3.

14. The extended release gastro retentive dosage form of claim 11, wherein said emulsifying agent is a surfactant.

15. The extended release gastro retentive dosage form of claim 14, wherein said surfactant is a nonionic, an anionic, a cationic, an amphoteric surfactant, or a combination thereof.

16. The extended release gastro retentive dosage form of claim 14, wherein said surfactant is selected from the group consisting of sodium lauryl sulfate, sorbitan stearate, sorbitan esters, sodium laureth sulfate, sarkosyl, cocamidopropyl betaine (CAPB), sodium lauryl ether sulfonate, alkyl benzene sulfonates, nonylphenol ethoxylate, hexadecylbetaine, lauryl betaine, and ether ethoxylate, Sodium Myristyl sulfate, polysorbate 20, polysorbate 80, lecithin, Octyl phenol ethoxylate (Triton X-100), glyceryl monostearate, 3-[(3-Cholamidopropyl)dimethylammonio]-1-propanesulfonate (CHAPS).

17. (canceled)

18. The extended release gastro retentive dosage form of claim 11, wherein said emulsifying agent is a cholate.

19. The extended release gastro retentive dosage form of claim 18, wherein said cholate is selected from the group consisting of cholic acid, glycocholic acid, taurocholic acid, deoxycholic acid, chenodeoxycholic acid, glycochenodeoxycholic acid, taurochenodeoxycholic acid and lithocholic acid.

20. The extended release gastro retentive dosage form of claim 11, wherein said pH value is obtained with a weak base.

21. The extended release gastro retentive dosage form of claim 20, wherein said weak base is a carbonate salt.

22. The extended release gastro retentive dosage form of claim 21, wherein said carbonate salt is selected from the group consisting of sodium carbonate (Na₂CO₃), potassium carbonate (K₂CO₃), sodium bicarbonate (NaHCO₃), and potassium and bicarbonate (KHCO₃).

23. The extended release gastro retentive dosage form of claim 11, further comprising a proton pump inhibitor.

24. The extended release gastro retentive dosage form of claim 23, wherein said proton pump inhibitor is omeprazole, lansoprazole, dexlansoprazole, rabeprazole, pantoprazole, esomeprazole, omeprazole, or combinations thereof.

25. The extended release gastro retentive dosage form of claim 11, further comprising an antibiotic.

26. The extended release gastro retentive dosage form of claim 25, wherein said antibiotic is amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

27.-36. (canceled)

37. A method for the prevention or treatment of Helicobacter pylori infection, gastric ulcers, gastric cancer or a combination thereof comprising administering to a subject in need thereof a therapeutically effective amount of the extended release gastro retentive dosage form of claim 11.

38. (canceled)

39. The method of claim 37, further comprising administering to said subject in need thereof a therapeutically effective amount of a proton pump inhibitor.

40. The method of claim 39, wherein said proton pump inhibitor is omeprazole, lansoprazole, dexlansoprazole, rabeprazole, pantoprazole, esomeprazole, omeprazole, or combinations thereof.

41. The method of any one of claim 37, further comprising administering to said subject in need thereof a therapeutically effective amount of an antibiotic.

42. The method of claim 41, wherein said antibiotic is amoxicillin, doxycycline, cephalexin, ciprofloxacin, clindamycin, metronidazole, azithromycin, clarithromycin, rifabutin, sulfamethoxazole and trimethoprim, amoxicillin and clavulanate, levofloxacin, and combinations thereof.

43. The method of any one of claim 39, wherein said proton pump inhibitor is administered before, or at the same time as said extended release gastro retentive dosage form: and/or
wherein said antibiotic is administered before, after, or at the same time as said extended release gastro retentive dosage form; or
wherein said proton pump inhibitor is administered before said extended release gastro retentive dosage form, and said antibiotic is administered at the same time or after said extended release gastro retentive dosage form.

44.-82. (canceled)
